SEQUENCE LISTING (54 sequences)

SEQ ID NO: 1 - RNA, 19 nt, Synthetic construct
  source 1..19: mol_type = other RNA
cgtcagtcctgtcatacgt

SEQ ID NO: 2 - RNA, 20 nt, Synthetic construct
  source 1..20: mol_type = other RNA
ggggtcgtagaaatacgcca

SEQ ID NO: 3 - RNA, 20 nt, Synthetic construct
  source 1..20: mol_type = other RNA
aagaaagtgggcatggaggg

SEQ ID NO: 4 - RNA, 20 nt, Synthetic construct
  source 1..20: mol_type = other RNA
cgccgcctaccacatgacgg

SEQ ID NO: 5 - RNA, 20 nt, Synthetic construct
  source 1..20: mol_type = other RNA
cgacgatgactcctcctcgg

SEQ ID NO: 6 - RNA, 20 nt, Synthetic construct
  source 1..20: mol_type = other RNA
aaagtaaagccattcgccag

SEQ ID NO: 7 - RNA, 20 nt, Synthetic construct
  source 1..20: mol_type = other RNA
gtccatggtcagggtcccgg

SEQ ID NO: 8 - RNA, 20 nt, Synthetic construct
  source 1..20: mol_type = other RNA
agtcaatgtagtcctcggag

SEQ ID NO: 9 - DNA, 21 bp, Synthetic construct
  source 1..21: mol_type = other DNA
aacttgccgtatgtccagatc

SEQ ID NO: 10 - DNA, 21 bp, Synthetic construct
  source 1..21: mol_type = other DNA
agagtccgatagagttacccg

SEQ ID NO: 11 - DNA, 21 bp, Synthetic construct
  source 1..21: mol_type = other DNA
gataatctgggagtgtgcagg

SEQ ID NO: 12 - DNA, 20 bp, Synthetic construct
  source 1..20: mol_type = other DNA
gtcagacgctgtagaaccag

SEQ ID NO: 13 - DNA, 21 bp, Synthetic construct
  source 1..21: mol_type = other DNA
cccagcactgagtacatcaac

SEQ ID NO: 14 - DNA, 22 bp, Synthetic construct
  source 1..22: mol_type = other DNA
gtatgtacgagaggcaacagtc

SEQ ID NO: 15 - DNA, 21 bp, Synthetic construct
  source 1..21: mol_type = other DNA
tgcctaatttggaggtgatcc

SEQ ID NO: 16 - DNA, 23 bp, Synthetic construct
  source 1..23: mol_type = other DNA
ttgtcagtgcccaatattttctg

SEQ ID NO: 17 - DNA, 21 bp, Synthetic construct
  source 1..21: mol_type = other DNA
agaacatccagagcttgaagg

SEQ ID NO: 18 - DNA, 22 bp, Synthetic construct
  source 1..22: mol_type = other DNA
caattttctgaaccaagggagc

SEQ ID NO: 19 - DNA, 23 bp, Synthetic construct
  source 1..23: mol_type = other DNA
cagaagtcatagccactctcaag

SEQ ID NO: 20 - DNA, 21 bp, Synthetic construct
  source 1..21: mol_type = other DNA
ctcctttccaggtcagttagc

SEQ ID NO: 21 - DNA, 21 bp, Synthetic construct
  source 1..21: mol_type = other DNA
tttgagaccatccagagcttg

SEQ ID NO: 22 - DNA, 22 bp, Synthetic construct
  source 1..22: mol_type = other DNA
ttcttgaccatccttgagagtg

SEQ ID NO: 23 - DNA, 21 bp, Synthetic construct
  source 1..21: mol_type = other DNA
cgcttgagtcggcaaagaaat

SEQ ID NO: 24 - DNA, 22 bp, Synthetic construct
  source 1..22: mol_type = other DNA
gtgcaagtctcatgaagtgagc

SEQ ID NO: 25 - DNA, 20 bp, Synthetic construct
  source 1..20: mol_type = other DNA
ggatcaaacgtggctgagcg

SEQ ID NO: 26 - DNA, 20 bp, Synthetic construct
  source 1..20: mol_type = other DNA
gattactcagggcccggctg

SEQ ID NO: 27 - DNA, 20 bp, Synthetic construct
  source 1..20: mol_type = other DNA
cgtgcccacgtcaaggagta

SEQ ID NO: 28 - DNA, 25 bp, Synthetic construct
  source 1..25: mol_type = other DNA
ttcgagtgacaaacacgacgactgc

SEQ ID NO: 29 - DNA, 22 bp, Synthetic construct
  source 1..22: mol_type = other DNA
ccacgtgttgagatcattgcca

SEQ ID NO: 30 - DNA, 20 bp, Synthetic construct
  source 1..20: mol_type = other DNA
tgcgtggtctcactccagtt

SEQ ID NO: 31 - DNA, 20 bp, Synthetic construct
  source 1..20: mol_type = other DNA
tgcagcccagatcacccaga

SEQ ID NO: 32 - DNA, 21 bp, Synthetic construct
  source 1..21: mol_type = other DNA
cagcagctgcagggccattag

SEQ ID NO: 33 - DNA, 20 bp, Synthetic construct
  source 1..20: mol_type = other DNA
agtcccaactcaacgccgac

SEQ ID NO: 34 - DNA, 22 bp, Synthetic construct
  source 1..22: mol_type = other DNA
ggaccccaactccacctctgga

SEQ ID NO: 35 - DNA, 20 bp, Synthetic construct
  source 1..20: mol_type = other DNA
tgccatgtttgacttcagcg

SEQ ID NO: 36 - DNA, 19 bp, Synthetic construct
  source 1..19: mol_type = other DNA
gttcttcagcaccagagcc

SEQ ID NO: 37 - DNA, 21 bp, Synthetic construct
  source 1..21: mol_type = other DNA
tcaagacgacataggacactt

SEQ ID NO: 38 - DNA, 23 bp, Synthetic construct
  source 1..23: mol_type = other DNA
ggacattggctaaaacaacagtg

SEQ ID NO: 39 - DNA, 23 bp, Synthetic construct
  source 1..23: mol_type = other DNA
ggtgaaatatattgtgcgtctcc

SEQ ID NO: 40 - DNA, 21 bp, Synthetic construct
  source 1..21: mol_type = other DNA
actaaaccctccatgatgtgc

SEQ ID NO: 41 - DNA, 20 bp, Synthetic construct
  source 1..20: mol_type = other DNA
tctgcaagtgttcgccatag

SEQ ID NO: 42 - DNA, 21 bp, Synthetic construct
  source 1..21: mol_type = other DNA
cagttttccttgtttccaccg

SEQ ID NO: 43 - DNA, 20 bp, Synthetic construct
  source 1..20: mol_type = other DNA
aaccgaagtcatagccacac

SEQ ID NO: 44 - DNA, 19 bp, Synthetic construct
  source 1..19: mol_type = other DNA
cctcccttctggtcagttg

SEQ ID NO: 45 - DNA, 20 bp, Synthetic construct
  source 1..20: mol_type = other DNA
cgcccaaaccgaagtcatag

SEQ ID NO: 46 - DNA, 21 bp, Synthetic construct
  source 1..21: mol_type = other DNA
cttctggtcagttggatttgc

SEQ ID NO: 47 - DNA, 21 bp, Synthetic construct
  source 1..21: mol_type = other DNA
aagtgtgaatgtaaggtcccc

SEQ ID NO: 48 - DNA, 21 bp, Synthetic construct
  source 1..21: mol_type = other DNA
gtgctccccttgttcagtatc

SEQ ID NO: 49 - DNA, 24 bp, Synthetic construct
  source 1..24: mol_type = other DNA
ggcgtttgagtcagcaaagaagtc

SEQ ID NO: 50 - DNA, 21 bp, Synthetic construct
  source 1..21: mol_type = other DNA
tcatggagtgggccatagctt

SEQ ID NO: 51 - DNA, 23 bp, Synthetic construct
  source 1..23: mol_type = other DNA
gtccacgtgttgagatcattgct

SEQ ID NO: 52 - DNA, 20 bp, Synthetic construct
  source 1..20: mol_type = other DNA
gcctctgtgtggtccatcct

SEQ ID NO: 53 - DNA, 20 bp, Synthetic construct
  source 1..20: mol_type = other DNA
acaccctgctgctttgccta

SEQ ID NO: 54 - DNA, 20 bp, Synthetic construct
  source 1..20: mol_type = other DNA
cgggtgacaaagacgactgc

(19) United States
(12) Patent Application Publication
(10) Pub. No.: US 2024/0398814 A1
(43) Pub. Date: Dec. 5, 2024

(54) KRAS INHIBITOR AND HDAC INHIBITOR COMBINATION FOR THE TREATMENT OF CANCER

(71) Applicant: Northwestern University, Evanston, IL (US)

(72) Inventors: Lillian Jennifer EICHNER, Evanston, IL (US); Reuben SHAW, Blue Hill, ME (US)

(21) Appl. No.: 18/734,662

(22) Filed: Jun. 5, 2024

Related U.S. Application Data
(60) Provisional application No. 63/506,274, filed on Jun. 5, 2023.

(51) Int. Cl. A61K 31/519; A61K 31/4406; A61P 35/00

(52) U.S. Cl. CPC A61K 31/519; A61K 31/4406; A61P 35/00

(57) ABSTRACT

Provided herein are pharmaceutical compositions comprising KRAS inhibitors and HDAC inhibitors and methods of administering KRAS inhibitors and HDAC inhibitors for the treatment/prevention of cancer. In particular, an HDAC inhibitor is administered to overcome KRAS inhibitor resistance in KRAS, LKB1 mutant lung cancer.

### STATEMENT REGARDING FEDERALLY SPONSORED RESEARCH OR DEVELOPMENT

This invention was made with government support under grant number CA251636 awarded by the National Institutes of Health. The government has certain rights in the invention.

### FIELD

Provided herein are pharmaceutical compositions comprising KRAS inhibitors and HDAC inhibitors and methods of administering KRAS inhibitors and HDAC inhibitors for the treatment/prevention of cancer. In particular, an HDAC inhibitor is administered to overcome KRAS inhibitor resistance in KRAS, LKB1 mutant lung cancer.

### CROSS-REFERENCE TO RELATED APPLICATIONS

This application claims the benefit of U.S. Provisional Application No. 63/506,274, filed Jun. 5, 2023, the content of which is herein incorporated by reference in its entirety.

### SEQUENCE LISTING STATEMENT

The text of the computer readable sequence listing filed herewith, titled “NWEST_42116_202_SequenceListing.xml”, created Jun. 4, 2024, having a file size of 48,588 bytes, is hereby incorporated by reference in its entirety.

### BACKGROUND

Targeted therapies have begun to prove themselves as successful treatments against cancer types harboring specific, defined vulnerabilities. However, only a small subset of tumor types have targeted therapies currently available, as such agents only exist for a limited number of oncogenic drivers. Moreover, tumors characterized by loss of tumor suppressor genes provide no clear targets against which to develop inhibitors. Transcriptional dependencies of tumors have emerged as definable and therapeutically-tractable liabilities that can be oncogene-agnostic (Ref. 1; incorporated by reference in its entirety). Much recent effort has focused on targeting epigenetic regulators (e.g. Brd4) as a means to globally affect transcription in such tumors (Refs. 2-6; incorporated by reference in their entireties). One case in point are Histone Deacetylase (HDAC) inhibitors, which were originally developed to antagonize the reduced global histone acetylation observed in many tumor types (Refs. 7-8; incorporated by reference in their entireties). Several HDAC inhibitors are now FDA approved to treat hematopoietic malignancies (Ref. 9; incorporated by reference in its entirety), although efficacy of HDAC inhibitors in solid tumors has been disappointingly limited. Recent efforts to identify effective approaches to HDAC inhibitor combination therapy have gained traction in specific tumor types (Refs. 10-14; incorporated by reference in their entireties). However, current FDA-approved inhibitors target multiple HDACs, and better therapeutic potential may be realized with more selective inhibitors aimed at one or two HDACs. Despite the fact HDAC inhibitors are already in the clinic, little analysis of disruption of the four Class I HDACs has been performed in genetically engineered tumor models in mice that might help to narrow down which are most important in different tumor contexts in vivo. Importantly, recent studies indicate that the HDAC inhibitor entinostat, which is selective to HDAC1 and HDAC3, exhibits potent effects in boosting therapeutic response in specific contexts (Refs. 15-18; incorporated by reference in their entireties).

However, even with increased understanding of their therapeutic potential, the molecular mechanisms that mediate tumor growth control by individual HDACs in vivo remain poorly understood.

There are 4 members of the Class I HDAC family: HDAC1, HDAC2, HDAC3, and HDAC8.

Histone Deacetylase 3 (HDAC3) is unique amongst them in requiring the Nuclear Receptor Co-Repressor (NCoR) complex for its enzymatic activity (Ref. 19; incorporated by reference in its entirety), forming a core complex of NCoR1/SMRT, TBL1X, TBL1XR1, GPS2, and HDAC3. HDAC3 has been shown to deacetylate histone and non-histone proteins, and can function in part through deacetylase-independent mechanisms. Tissue-specific deletion of HDAC3 in metabolic tissues in mice has identified striking biological functions and deregulation of distinct non-overlapping transcriptional programs unique to each corresponding tissue. Collectively, these studies reveal that HDAC3 function is not uniformly through global control of histone acetylation, but is nuanced and directed in a tissue-specific fashion. For example, HDAC3 deletion in brown adipose tissue causes mice to become hypothermic and succumb to acute cold exposure (Ref. 20; incorporated by reference in its entirety), but HDAC3 deletion in the liver induces hypertrophy and metabolic alterations (Refs. 21-23; incorporated by reference in their entireties), and the genes controlled by HDAC3 in each tissue are distinct and relate to tissue-specific functions. Despite clinical advancement of inhibitors of Class I HDACs as therapeutics, any potential role of HDAC3 in tumorigenesis remains largely unknown, as its in vivo function and mechanism of action has predominantly been examined in metabolic tissues.

The Liver Kinase B1 (LKB1/STK11) tumor suppressor is mutated in ˜20% of lung adenocarcinoma, often concurrently with Kristen rat sarcoma viral oncogene (Kras) mutation (Refs. 24-27; incorporated by reference in their entireties). LKB1 is a serine/threonine kinase that directly activates a family of 14 downstream kinases in the AMP-activated protein kinase (AMPK) family (Ref. 28; incorporated by reference in its entirety). Recent studies dissecting which of these kinases are most critical to LKB1 tumor suppressor function in the lung revealed that in fact AMPK may not be as critical as originally hypothesized (Ref. 29; incorporated by reference in its entirety), and that two AMPK-related kinases, SIK1 and SIK3, are the most important for suppressing growth of lung tumors (Ref. 30-31; incorporated by reference in their entireties). Notably, only two sets of substrates of the SIK⅓ kinases are well-established: the CREB Regulated Transcription Coactivator (CRTC) family of CREB co-activators and the Class IIa family of HDACs (32). Class IIa HDACs, which lack catalytic activity themselves, are hypothesized to function as transcriptional co-regulators of HDAC3 (Refs. 33-34; incorporated by reference in their entireties).

### SUMMARY

Provided herein are pharmaceutical compositions comprising KRAS inhibitors and HDAC inhibitors and methods of administering KRAS inhibitors and HDAC inhibitors for the treatment/prevention of cancer. In particular, an HDAC inhibitor is administered to overcome KRAS inhibitor resistance in KRAS, LKB1 mutant lung cancer.

In some embodiments, provided herein are methods of treating or preventing cancer in a subject comprising co-administering to the subject a Kirsten rat sarcoma (KRAS) inhibitor and a histone deacetylase (HDAC) inhibitor.

In some embodiments, provided herein is an HDAC inhibitor for use with a KRAS inhibitor in treating or preventing cancer.

In some embodiments, provided herein is a KRAS inhibitor for use with a HDAC inhibitor in treating or preventing cancer.

In some embodiments, provided herein are a HDAC inhibitor and a KRAS inhibitor for use in treating or preventing cancer.

In some embodiments, provided herein are pharmaceutical compositions comprising a KRAS inhibitor and a HDAC inhibitor.

In some embodiments, a subject suffers from lung cancer. In some embodiments, the lung cancer is a non-small cell lung cancer (NSCLC). In some embodiments, the cancer is KRAS inhibitor resistant. In some embodiments, the cancer is trametinib resistant. In some embodiments, the cancer is KRAS, LKB1 mutant lung cancer.

In some embodiments, the KRAS inhibitor is selected from Sotorasib (AMG510), Adagrasib/MRTX849, AMG 404, trametinib, RMC-4630, afatinib, pembro, panitumumab, carbo/pem/docetaxel, everolimus, Palbociclib, bevacizumab, LY3537982, abemaciclib, erlotinib, sintilimab, temuterkib, LY3295668, cetuximab, JNJ-74699157 (ARS-3248), GDC-6036, atezo, spartalizumab, TNO155, EGF816 (nazertinib/EGFR TKI, RMC-4630, cobimetinib/Osimertinib, BI 1701963, MRTX1133, AMG510, and irinotecan. In some embodiments, the KRAS inhibitor is trametinib.

In some embodiments, the HDAC inhibitor is an HDAC3 inhibitor. In some embodiments, the HDAC inhibitor is selected from trichostatin A, vorinostat, givinostat, abexinostat, belinostat, panobinostat, resminostat, quisinostat, depsipeptide, entinostat, mocetinostat suberoyl bis-hydroxamic acid, scriptaid, apicidin, CBHA, CI 994, Salermide, Belinostat, KD 5170, MS-275, TC-H 106, Droxinostat, Mocetinostat, PCI-24781, Pimelic Diphenylamide 106, BRD3308, and RGFP966. In some embodiments, the HDAC inhibitor is entinostat.

### BRIEF DESCRIPTION OF THE DRAWINGS

FIGS. 1A-1F. HDAC3 is essential for lung tumorigenesis in vivo in KL and KP GEMM models of NSCLC. (A) Schematic of experimental design in Kras^{G12D/+}, LKB^{L/L} (KL) and KL-HDAC3^{L/L} (KL-HDAC3) mouse models administered lentivirus expressing Cre recombinase (Lenti-Cre). (B) Representative H&E-stained sections from the late timepoint. Scale bar 1000 um. (C) Quantitation from H&E-stained sections from the late timepoint cohort: Tumor area as a percentage of total lung area per mouse (n=10), tumor number per mouse (n=10), and average tumor size (n=482 or 230 as indicated). (D) Schematic of experimental design in Kras^{12D/+}, p53^{L/L} (KP) and KP-HDAC3^{L/L} (KP-HDAC3) mouse models administered Lenti-Cre. (E) Representative H&E-stained sections. Scale bar 1000 um. (F) Quantitation from H&E-stained sections: Tumor area as a percentage of total lung area per mouse (n=9 or 6 as indicated), tumor number per mouse (n=9 or 6 as indicated), and average tumor size (n=115 or 33 as indicated). Values are expressed as mean±s.e.m. * p-value<0.05, **** p-value<0.0001 determined by two-tailed Mann-Whitney test.

FIGS. 2A-2D. HDAC3 genome occupancy in primary tumors. (A) 1522 HDAC3 ChIP-seq peaks common to KL and KP primary tumors. (B) Example of HDAC3 ChIP-seq peaks at genomic regions bound by HDAC3 in both KL and KP primary tumors. (C) Heatmap of RNA-seq data showing FPKM read counts from primary tumors from LKB1 WT (Kras, KP) and LKB1 KO (KL, KPL) models for the 753 non-redundant genes associated with at least one HDAC3 ChIP-seq peak within 25kb of the TSS. FPKM, fragments-per-kilobase-of-transcript-per-million. Kra, Kras^{LSL-G12D/+}; KL Kras^{LSL-G12D/+}Stk11^{-/-}; KP Kras^{LSL-G12D/+}p53^{-/-}; KPL, Kras^{LSL-G12D/+}Stk11^{-/-} p53′. (D) Homer de novo motif enrichment analysis of the HDAC3-bound peaks in FIG. 2A. All significantly enriched motifs are listed.

FIGS. 3A-3C. HDAC3 cooperates with NKX2-1 to regulate the expression of a common set of target genes. (A) Western blot analysis of HDAC3, NKX2-1, or FGFR1 knockout (KO) by CRISPR/Cas9 in polyclonal lysates from KL LJE1 cells. (B) Plot of fold change upon HDAC3 KO compared to NKX2-1 KO for the genes significantly deregulated (adj. p-value<0.05, fold+1-0.5) upon loss of both factors in KL LJE1 cells. (C) Heatmap of RNA-seq data showing FPKM read counts for genes commonly upregulated (left) or downregulated (right) upon both HDAC3 KO and NKX2-1 KO in KL cells, as defined from red box regions on heatmap in FIG. 8D.

FIGS. 4A-4G. HDAC3 and NKX2-1 common target genes are aberrantly engaged upon Trametinib resistance. (A) Western blot analysis of protein lysates from KL LJE1 cells treated with vehicle, 10 nM trametinib, or 1 uM entinostat for 3 or 13 days. (B) Heatmap of RNA-seq data showing FPKM read counts across all treatment conditions for the 2,141 genes significantly upregulated (adj. p-value<0.05, fold>+1-0.5) upon 13 day trametinib compared to 13 day vehicle in KL LJE1 cells. Veh, vehicle; Tram, trametinib; Ent, entinostat. Red boxes identify TIER genes. (C) Nkx2-1 mRNA levels (FPKM) across all treatment conditions (n=3) from RNA-seq data in (B). (D) GSEA analysis of the 285 Trametinib-Induced, Entinostat-Reversed (“TIER”) genes queried across RNA-seq data from NKX2-1 KO vs NT KL LJE1 cells. (E) Heatmap of RNA-seq data showing FPKM read counts across all treatment conditions for the 112 TIER genes which are HDAC3 ChIP-seq target genes. Veh, vehicle; Tram, trametinib; Ent, entinostat. (F) Avpil mRNA levels across all treatment conditions from RNA-seq data from cells in (B) (n=3). Veh, vehicle; Tram, trametinib; Ent, entinostat. (G) HDAC3 ChIP-seq data in NT and HDAC3 KO KL LJE1 cells at the Avpil genomic locus. Values are expressed as mean±s.e.m. ** p-value<0.01, *** p-value<0.001, **** p-value<0.0001 determined by two-tailed student's t-test.

FIGS. 5A-5F. Trametinib plus entinostat combination treatment elicits therapeutic efficacy in KL NSCLC GEMM in vivo. (A) Average longitudinal BLI data. (B) Representative H&E-stained sections at experimental endpoint. Scale bar 1000 um. (C-E) Quantitation from H&E-stained sections: (C) tumor area as a percentage of total lung area per mouse, (D) average tumor size, and (E) tumor number per mouse. (F) Model of HDAC3 cooperation with NKX2-1 to support KL tumor growth basally and in the context of trametinib resistance. Values are expressed as mean±s.e.m. * p-value<0.05, ** <0.01, **** <0.0001 determined by t-test with Welch's correction.

FIGS. 6A-6D. HDAC3 deletion in vivo impairs tumor growth in KL and KP GEMM models of NSCLC. (A) Average longitudinal BLI data from the KL-HDAC3 experiment. (B) Representative H&E-stained sections from the KL-HDAC3 early timepoint. Scale bar 1000 um. (C) Quantitation from H&E-stained sections from the early timepoint cohort: Tumor area as a percentage of total lung area per mouse (n=10), tumor number per mouse (n=10), and average tumor size (n=227 or 100 as indicated). (D) Average longitudinal BLI data from the KP-HDAC3 experiment. Values are expressed as mean±s.e.m. ** p-value<0.01, *** p-value<0.001, p-value<0.0001 determined by two-tailed Mann-Whitney test.

FIGS. 7A-7B. HDAC3 genome occupancy in primary tumors. (A) Schematic of HDAC3 ChIP-seq experimental design in primary KL and KP tumors. (B) Plot of RNA-seq differential expression between KL versus Kras primary tumors for the HDAC3 target genes in FIG. 2A.

FIGS. 8A-8J. HDAC3 and NKX2-1 regulate the expression of a common set of target genes. (A) GSEA plots for genes deregulated (downregulated, top plot; upregulated, bottom plot) upon tamoxifen™-mediated in vivo deletion of NKX2-1 in Kras tumors (Snyder et al. Mol Cell, 2013 (44)) queried across HDAC3 KO RNA-seq data from KL LJE1 cells. NT, Non-Targeting control. (B) Heatmap of RNA-seq data showing FPKM read counts from NT or HDAC3 KO cells for genes deregulated upon NKX2-1 KO (adj. p-value<0.05, fold+1-0.5) in KL LJE1 cells. (C) 3,728 HDAC3 ChIP-seq peaks identified in KL LJE1 NT cells. Peaks were called using Input from NT cells, and HDAC3 ChIP-seq from HDAC3 KO cells as background. (D) Heatmap of RNA-seq data showing FPKM read counts from NT or NKX2-1 KO KL LJE1 cells for the genes deregulated upon HDAC3 KO (adj. p-value<0.05, fold+1-0.5). (E) Proliferation assessment of KL LJE1 cells after 5 days (n=6). (F) Western blot analysis from KP T3 cells. (G) Proliferation assessment of KP T3 cells after 5 days (n=6). (H) Heatmap of RNA-seq data showing FPKM read counts from NT or NKX2-1 KO cells for the genes deregulated upon HDAC3 KO (adj. p-value<0.05, fold+1-0.5) in KP T3 cells. 159/254 genes were strongly upregulated upon HDAC3 KO or NKX2-1 KO (red box, left), whereas 68/134 genes were mildly downregulated upon HDAC3 KO or NKX2-1 KO (red box, right). (I) Overlap of genes significantly upregulated or downregulated upon both HDAC3 KO and NKX2-1 KO in KP T3 (KP) versus KL LJE1 (KL) cells. (J) Fgfr1 mRNA levels (FPKM) from RNA-seq data across Kras (K) (n=9), KP (n=8), KL (n=9), and KPL (n=15) primary tumor (n=8-15). Values are expressed as mean±s.e.m. * p-value<0.05, ** <0.01, *** <0.001, **** p-value<0.0001 determined by two-tailed student's t-test.

FIGS. 9A-9L. HDAC3 and NKX2-1 common target genes are aberrantly engaged upon trametinib resistance. (A) Western blot analysis from KL LJE1 cells treated with vehicle, 10 nM trametinib, or 1 uM entinostat for 13 days. (B) qRT-PCR for Fgfr1 mRNA expression in KL LJE1 cells treated as in (A) (n=3). (C) Western blot analysis from human A549 cells treated for 9 days with vehicle (V) or 10 nM trametinib, and during the last 6 days co-treated with V or entinostat (Ent) at the doses indicated. (D) Western blot analysis of NT or HDAC3 KO KL LJE1 cells treated with vehicle or 10 nM trametinib for 10 days. (E) Proliferation assessment after 6 days of treatment with vehicle or 10 nM trametinib in NT, HDAC3 KO, or NKX2-1 KO KL LJE1 cells (n=6). (F-G) Western blot (F) and qRT-PCR (G) analysis from cells with (+) or without (−) LKB1 re-expression treated for 13 days; 10 nM trametinib (Tram), 1 uM entinostat (Ent). (H) Western blot analysis of protein lysates from KP T3 cells treated with vehicle, 10 nM trametinib, or 1 uM entinostat for 13 days. (I) Heatmap of RNA-seq data showing FPKM read counts for TIER genes differentially expressed between NT or NKX2-1 KO KL LJE1 cells. (J) Heatmap of RNA-seq data showing FPKM read counts from Kras (n=9), KP (n=8), KL (n=9), and KPL (n=15) primary tumors for the 112 TIER genes which are HDAC3 ChIP-seq target genes (FIG. 4E). (K) qRT-PCR for Avpil mRNA expression in KL LJE1 cells (n=3). (L) Overall survival data from patients with Kras amplified or G12-mutant tumors, comparing tumors with (n=8) or without (n=92) high AVP11 mRNA expression in the Firehose Legacy LUAD TCGA dataset.

Values are expressed as mean±s.e.m. ** <0.01, *** <0.001, **** p-value<0.0001 determined by two-tailed student's t-test.

FIG. 10. In vivo trametinib plus entinostat combination treatment in KL NSCLC GEMM. Schematic of experimental design. Lung tumors were initiated in Kras^{12D/+}, LKB1^{L}m (KL) mice by Lenti-Cre administration and mice were imaged weekly (BLI) starting 4 weeks post-Lenti-Cre. Treatment was initiated 34 days post-Lenti-Cre. Mice were treated for 42 days with vehicle, entinostat (Ent, 10 mg/kg), trametinib (Tram, 1 mg/kg), or entinostat plus trametinib (Ent+Tram) administered by oral gavage.

FIGS. 11A-11G. HDAC3 target genes common to KL and KP NSCLC cells are associated with p65 NF-kB and SASP pathways. (A) Western blot analysis of HDAC3 deletion (KO) by CRISPR/Cas9 using two different sgRNAs (g1, g2) in polyclonal lysates in KL LJE1 cells. (B) Overlap of genes upregulated upon HDAC3 KO using all sgRNAs tested compared to Non-Targeting (NT) control using RNA-seq data from two KL cells lines (LJE1, LJE7) and two KP cell lines (T3, 634T) using adj. p-value<0.05 and fold change >+/−0.5 cut-offs. (C) Enrichr Pathway and Transcription analysis of the 26 commonly upregulated genes identified in FIG. 1B. (D) Gene Set Enrichment Analysis (GSEA) plots of the “Hallmark TNFa Signaling Via NFkB” and “SASP Fridman Senescence” gene sets queried against RNA-seq data comparing HDAC3 KO vs NT conditions across KL LJE1 and KL LJE7 cells combined. (E) Plot of RNA-seq data from KL LJE1 cells (HDAC3 KO vs NT) for the 468 genes associated with at least one HDAC3 ChIP-seq peak identified in FIG. 17F. (F) Homer de novo motif enrichment analysis of the genomic regions bound by HDAC3 in ChIP-seq for the genes upregulated upon HDAC3 KO (red box, FIG. 11E). Motifs enriched uniquely among upregulated genes are shown. Red bars indicate statistically significant motifs. (G) HDAC3 ChIP-seq, H3K27ac ChIP-seq, H3K9ac ChIP-seq, and Input tracks in NT and HDAC3 KO KL LJE1 cells at the Cxc11, Cxc13, and Cxc15 genomic loci.

FIGS. 12A-12D. HDAC3 represses chemokine gene expression in NSCLC cells. (A) Fragments Per Kilobase of transcript per Million mapped reads (fpkm) values from RNA-seq data. Gene expression was assessed in a polyclonal population of KL LJE1 cells transduced by lentiviral infection to express an NT or HDAC3 directed sgRNA (HDAC3 KO) (FIG. 11A) (n=3). (B) qRT-PCR from KL LJE1 cells treated with vehicle (Veh), 2 uM entinostat (Ent), or 10uM RGFP966 (RGFP) for 72 hr (n=3). (C) Cytokines detected in the media of KL LJE1 cells after 3 days of culture as determined with the Mouse XL Cytokine Array ELISA. 112 cytokines were spotted, and all cytokines that were detected in the media are shown. Data indicate protein expression differences between NT and HDAC3 KO KL LJE1 cells (n=4). Red lines indicate+/−0.5 fold change cut-off. (D) Overlap of HDAC3-dependent targets in KL LJE1 cells identified by RNA-seq (FIG. 2A, yellow), or by ELISA (FIG. 2D, blue) using p<0.05 and +/−0.5 fold cut-offs. Bold indicates targets that can be readily detected at the mRNA level. Values are expressed as mean±s.e.m. * p-value<0.05, ** p-value<0.01, *** p-value<0.001, **** p-value<0.0001 as determined by two-tailed student's t-test with Welch's correction.

FIGS. 13A-13E. p65 NF-kB for HDAC3 repression of chemokine genes in NSCLC cells. (A) Western blot detecting p65 protein in nuclear or cytoplasmic fractions from KL LJE1 cells after 6 hr Vehicle, 2 uM entinostat, 10uM RGFP966, or 0.5 uM TSA treatment. (B) Western blot analysis of protein lysates from KL LJE1 cells transiently transfected with Flag-p65 and treated 6 hr with vehicle or 2 uM entinostat. (C) Western blot analysis of CRISPR/Cas9-mediated p65 deletion in polyclonal lysates from KL LJE1 HDAC3 KO cells. (D) qRT-PCR on NT or HDAC3 KO KL LJE1 cells +/−p65 KO (n=3). (E) Heatmap showing fpkm mapped read counts of the gene cluster upregulated upon HDAC3 KO in a p65-dependent manner from RNA-seq data from cells in FIG. 13C. Values are expressed as mean±s.e.m. * p-value<0.05, ** p-value<0.01, *** p-value<0.001, **** p-value<0.0001 as determined by two-tailed student's t-test with Welch's correction.

FIGS. 14A-14F. HDAC3 represses the HRCG transcriptional program in vivo in the KL GEMM of NSCLC. (A) Representative images of Cxc15 IHC in KL and KL-HDAC3 tumors.

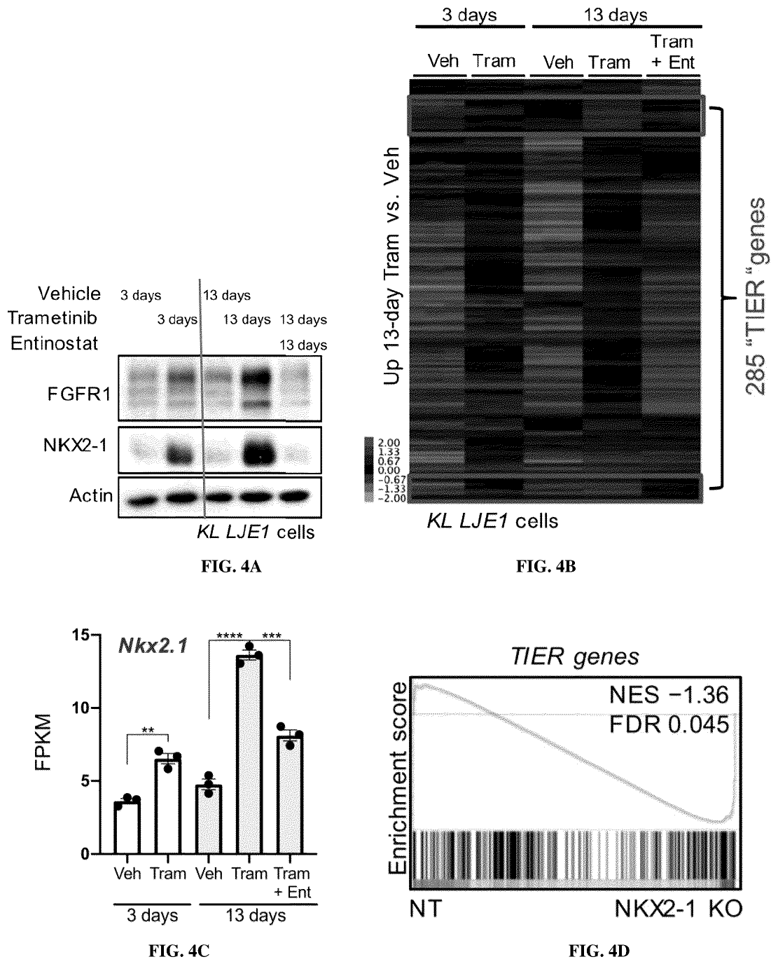

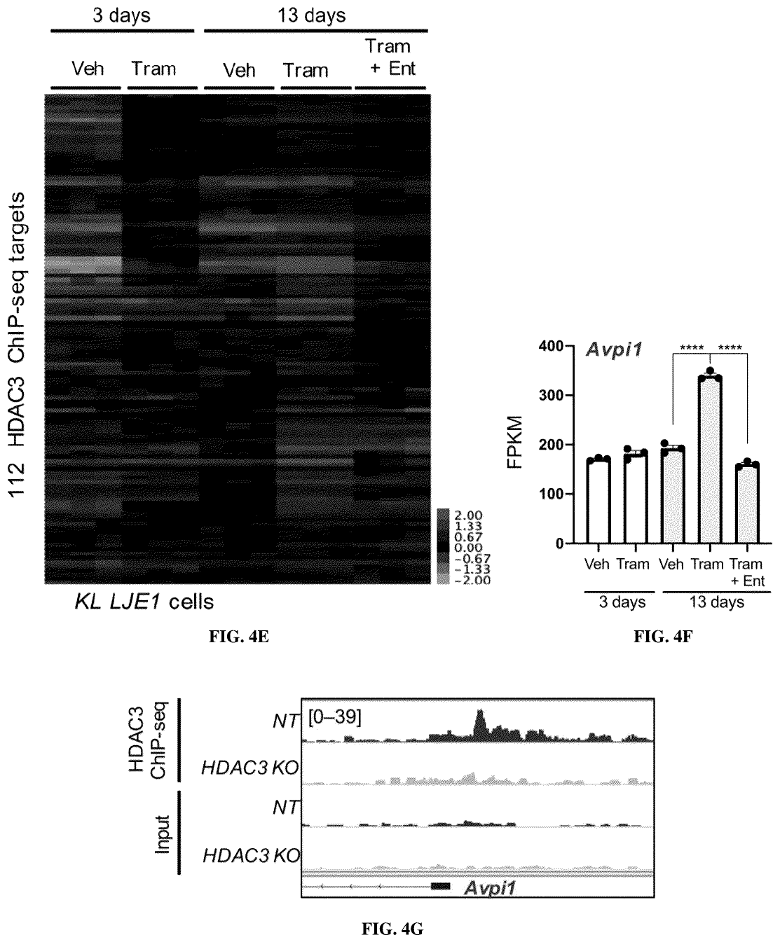

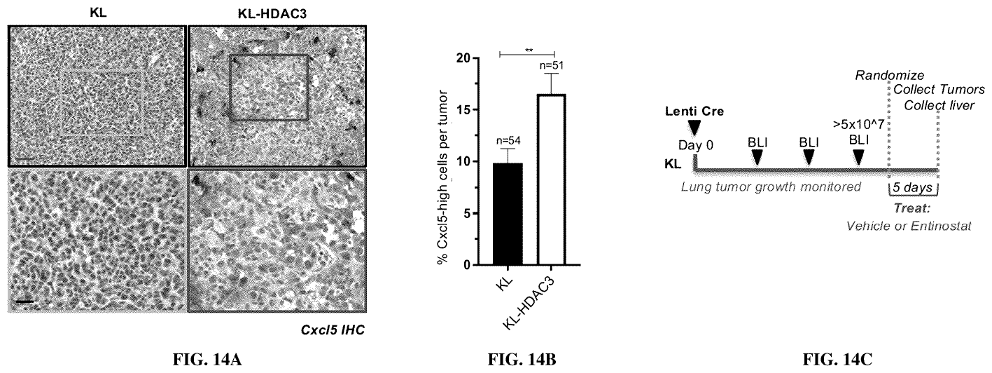

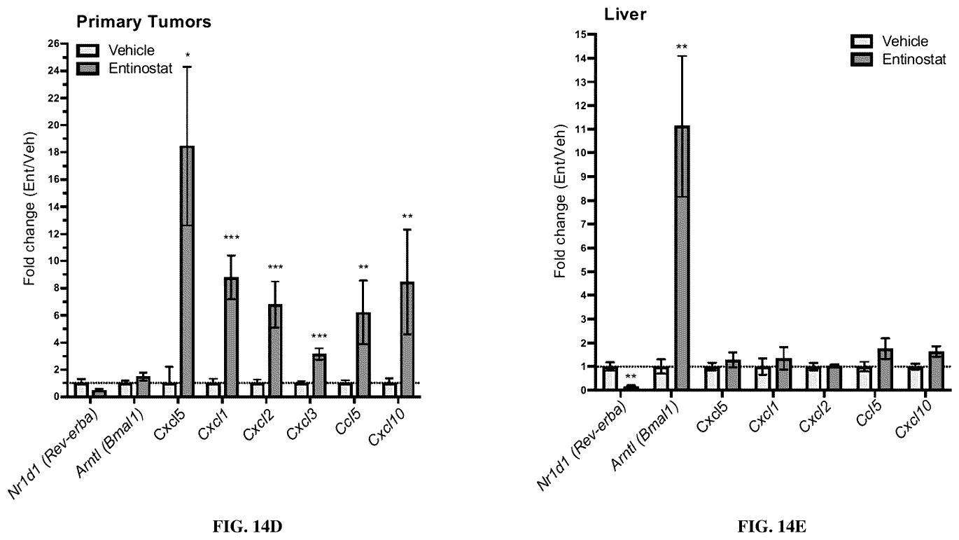

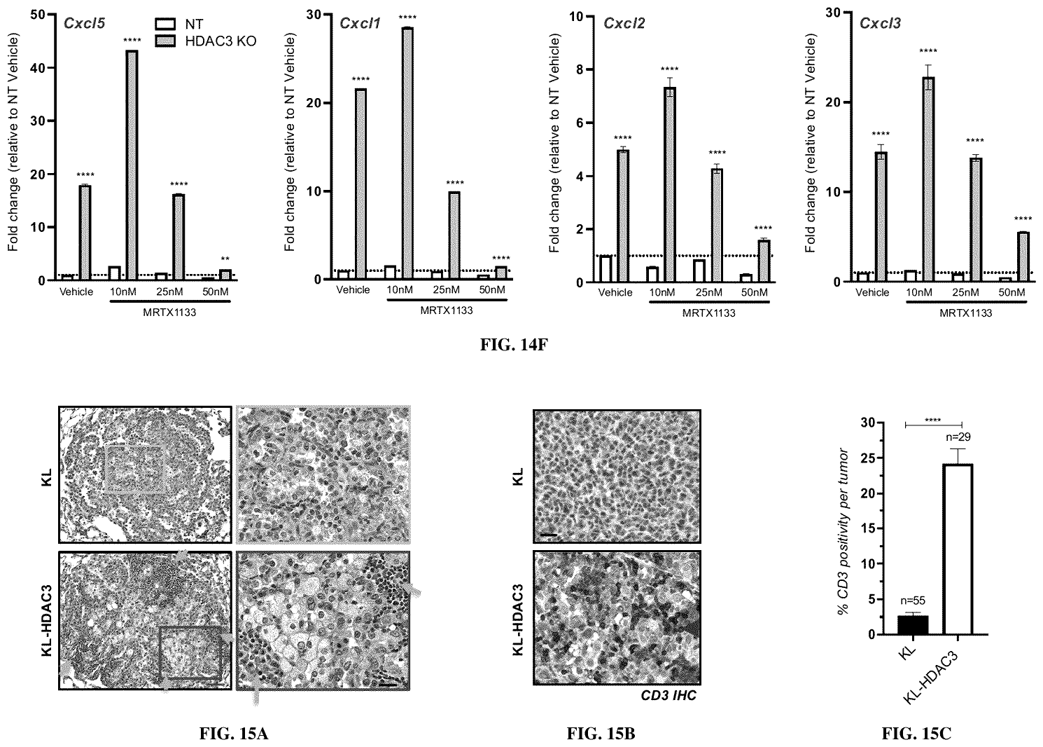

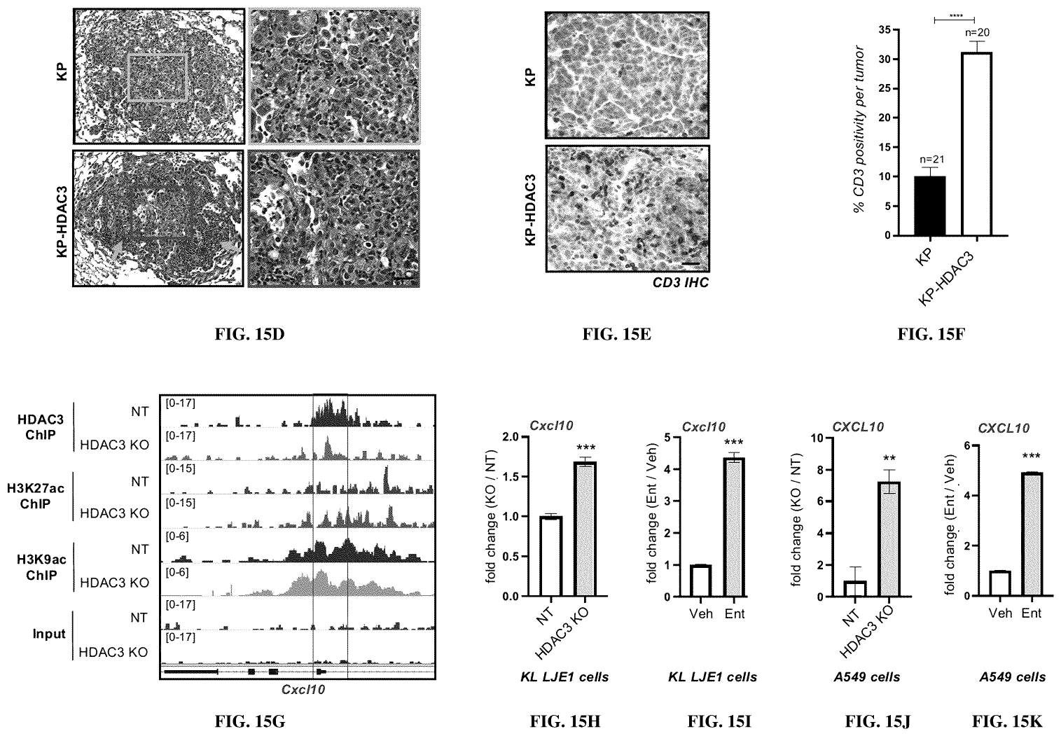

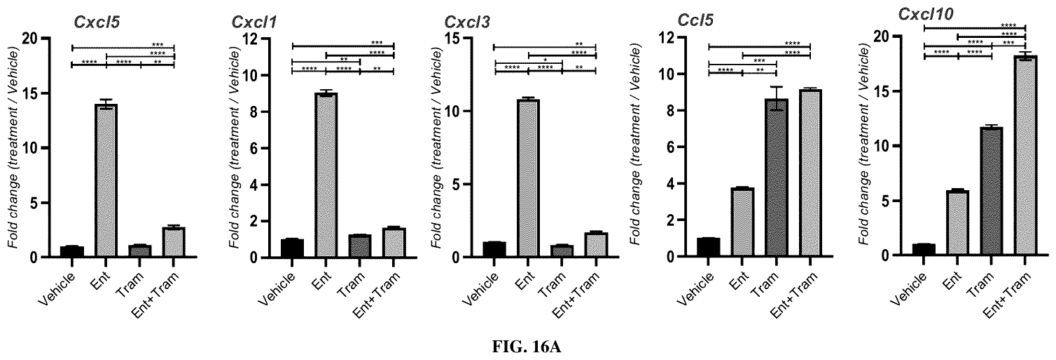

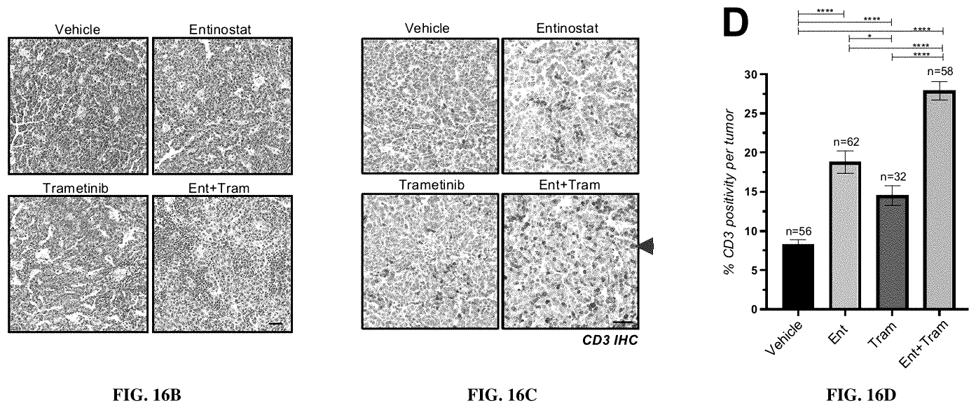

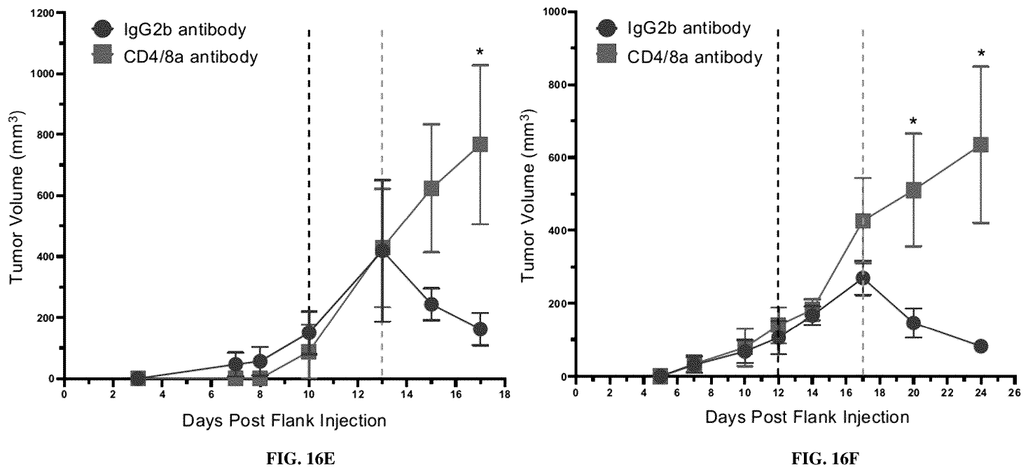

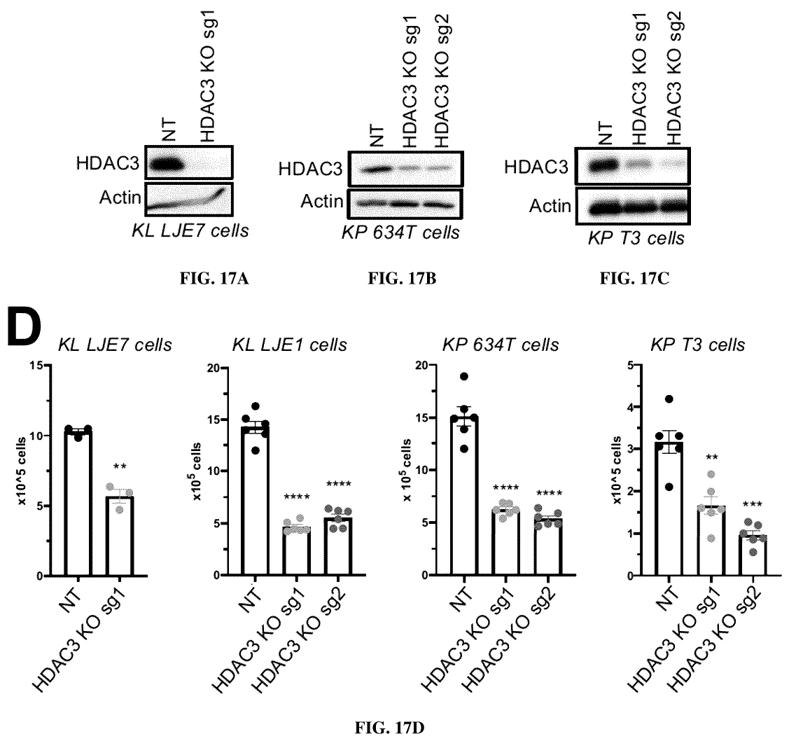

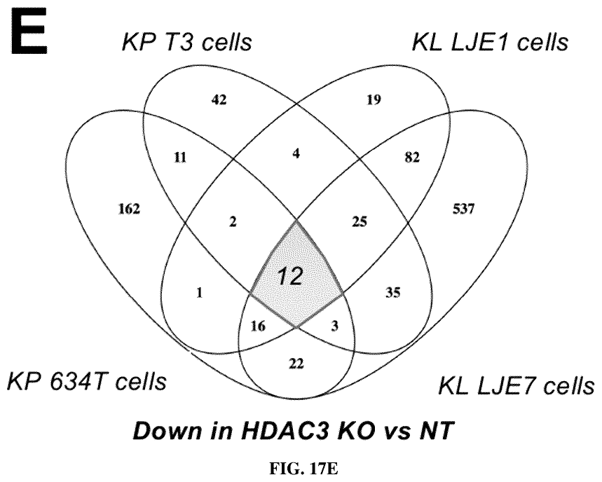

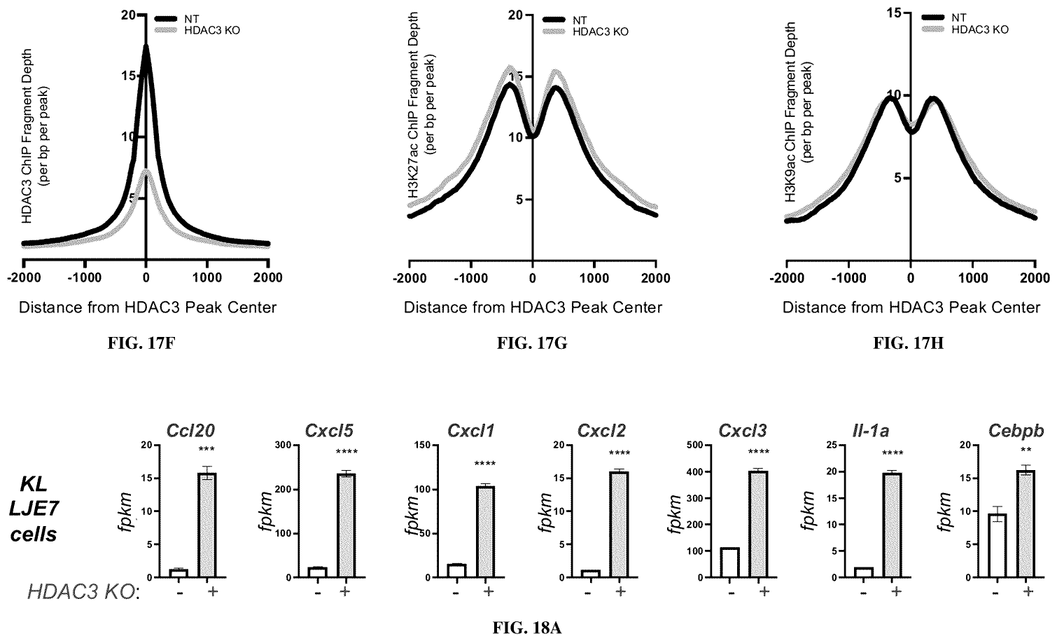

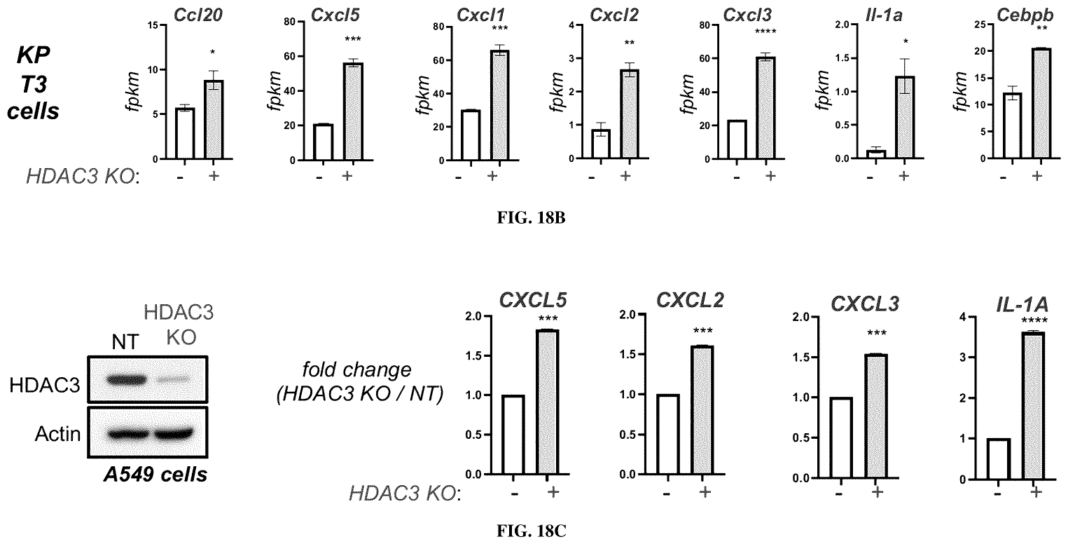

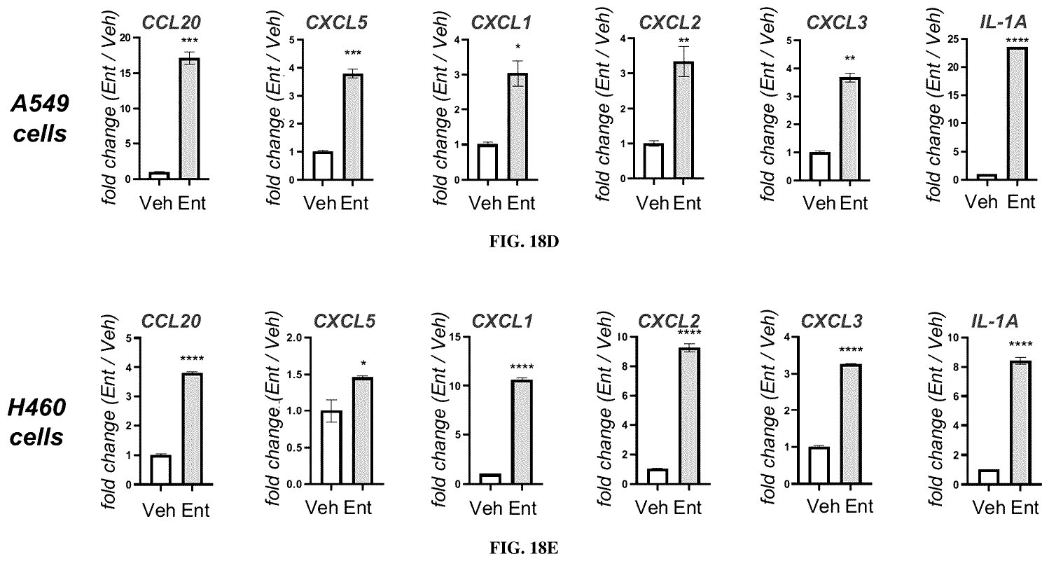

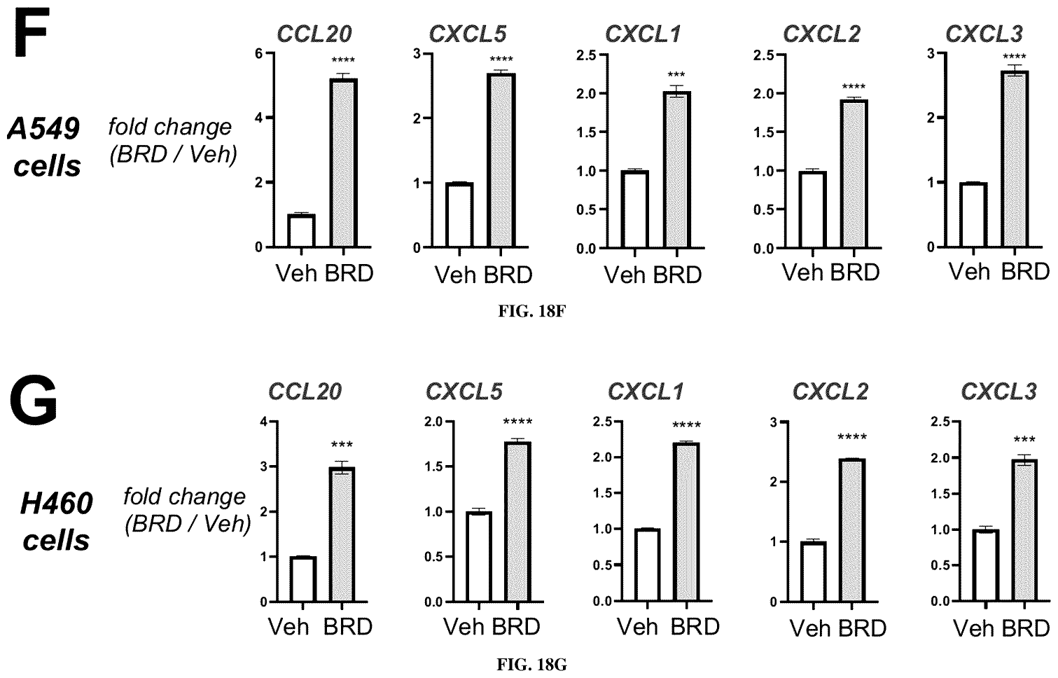

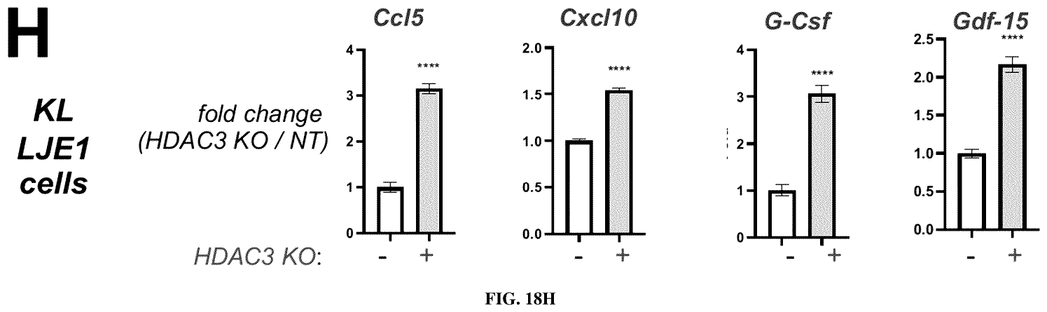

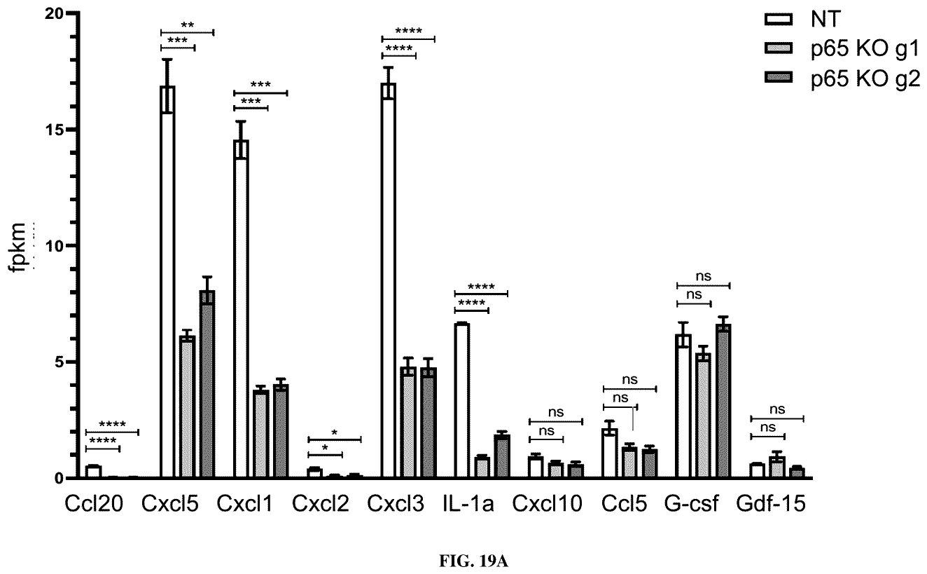

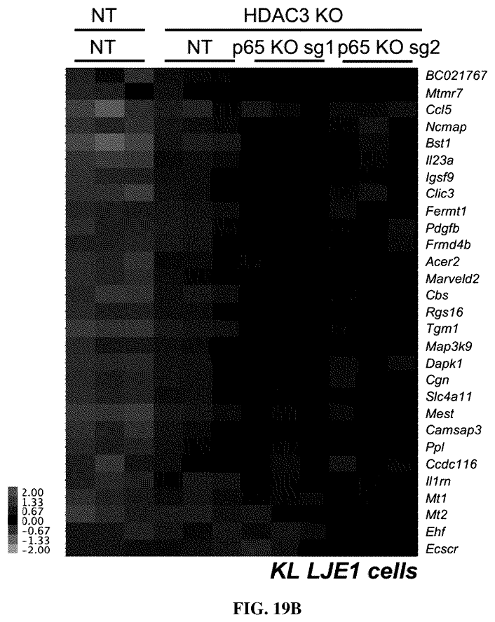

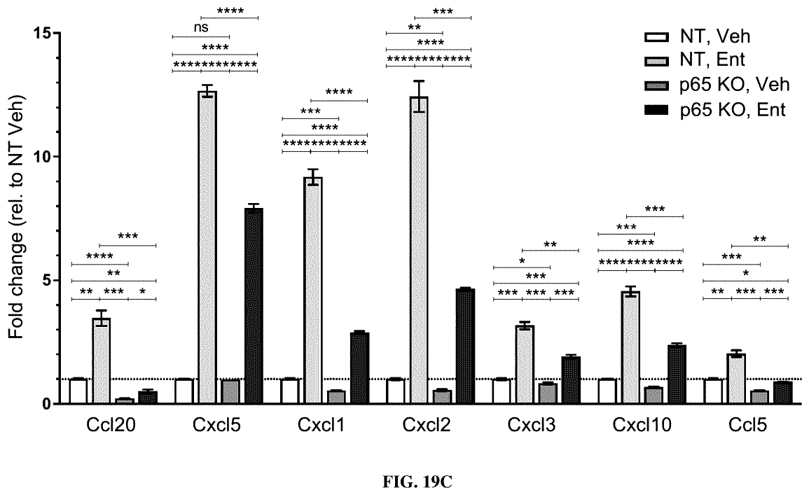

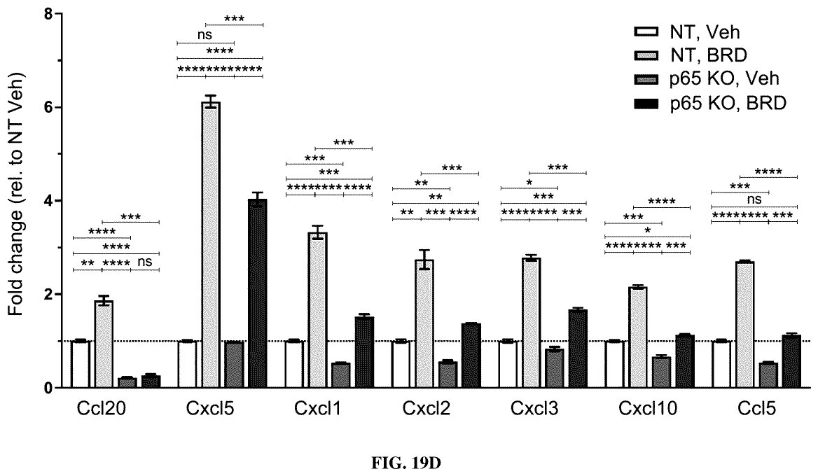

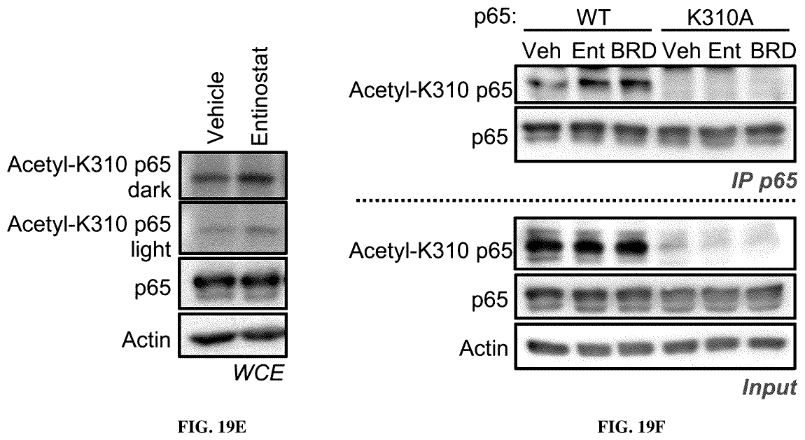

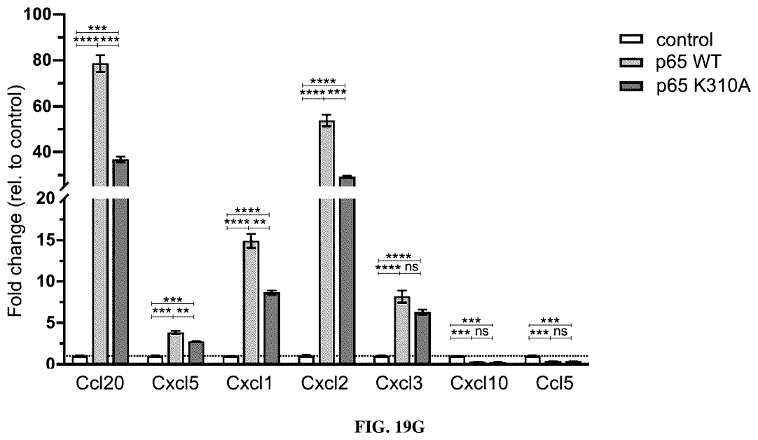

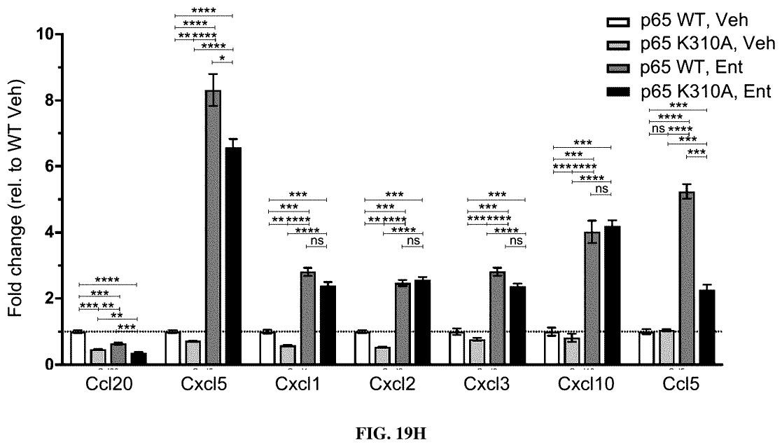

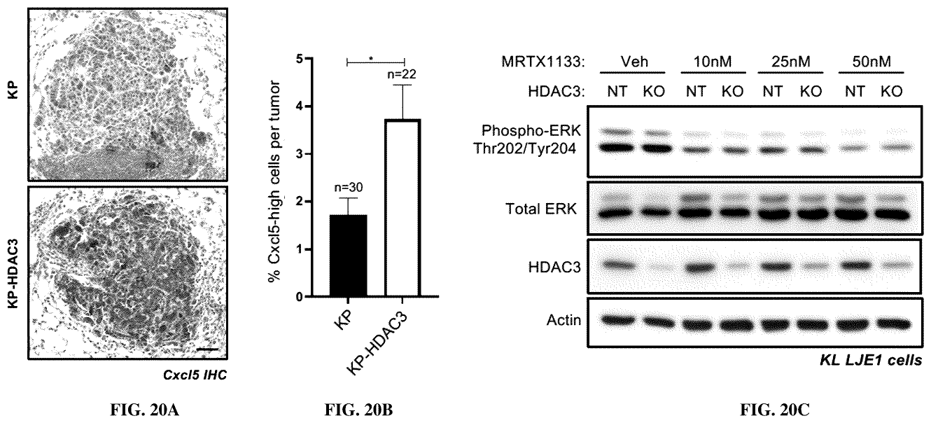

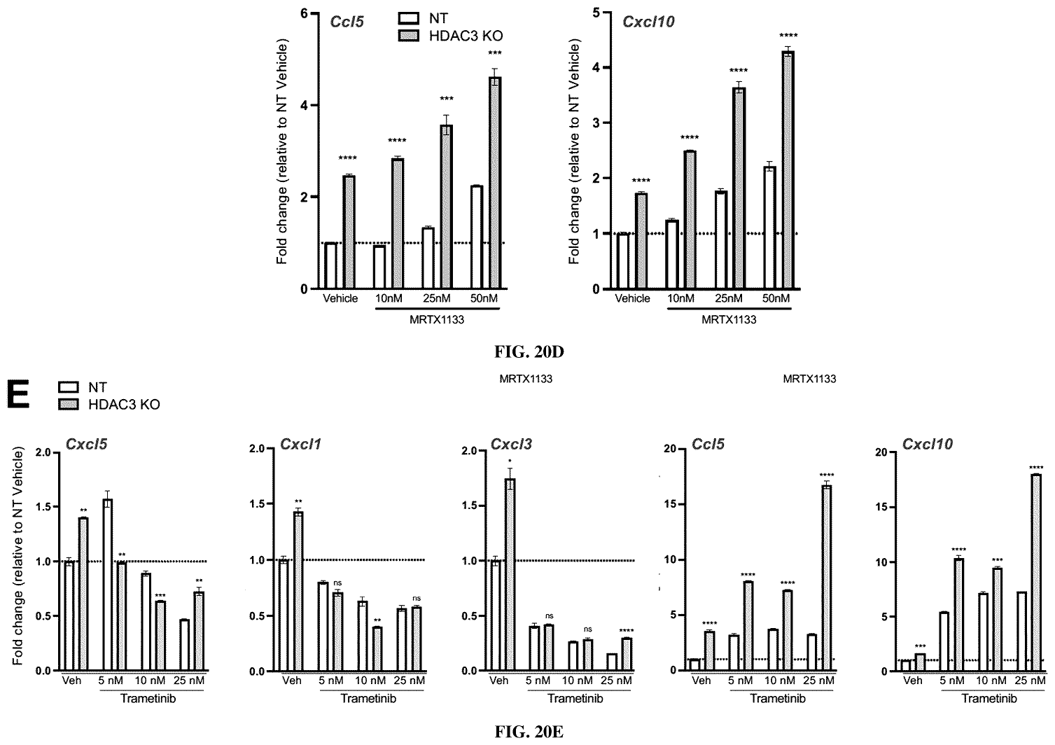

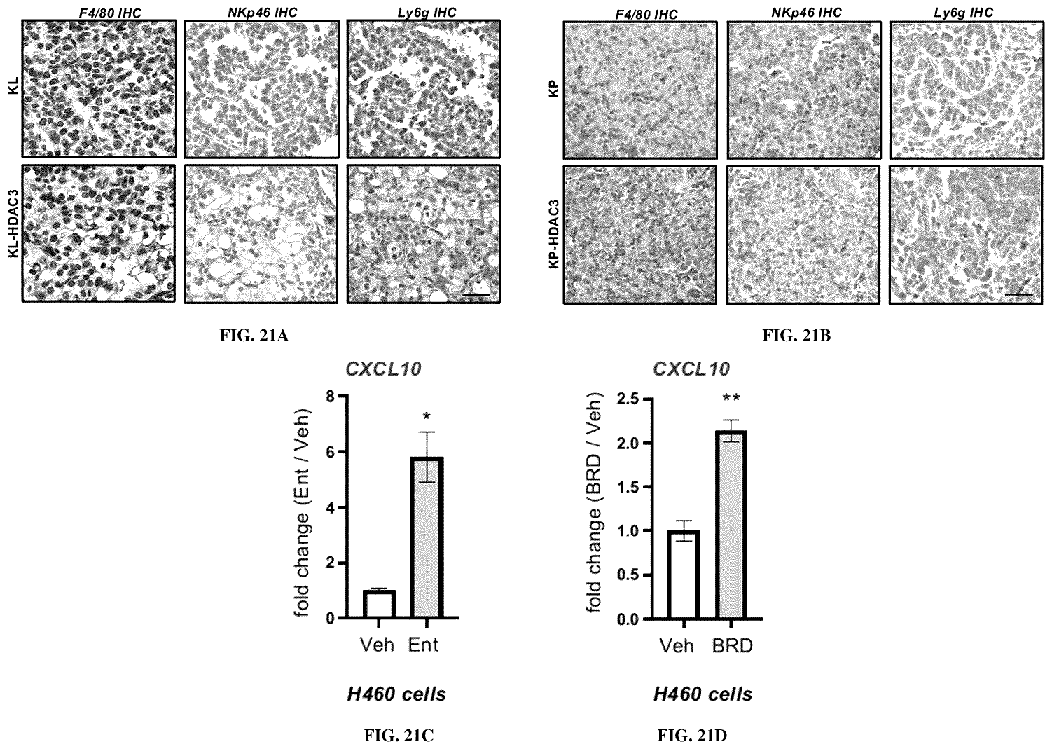

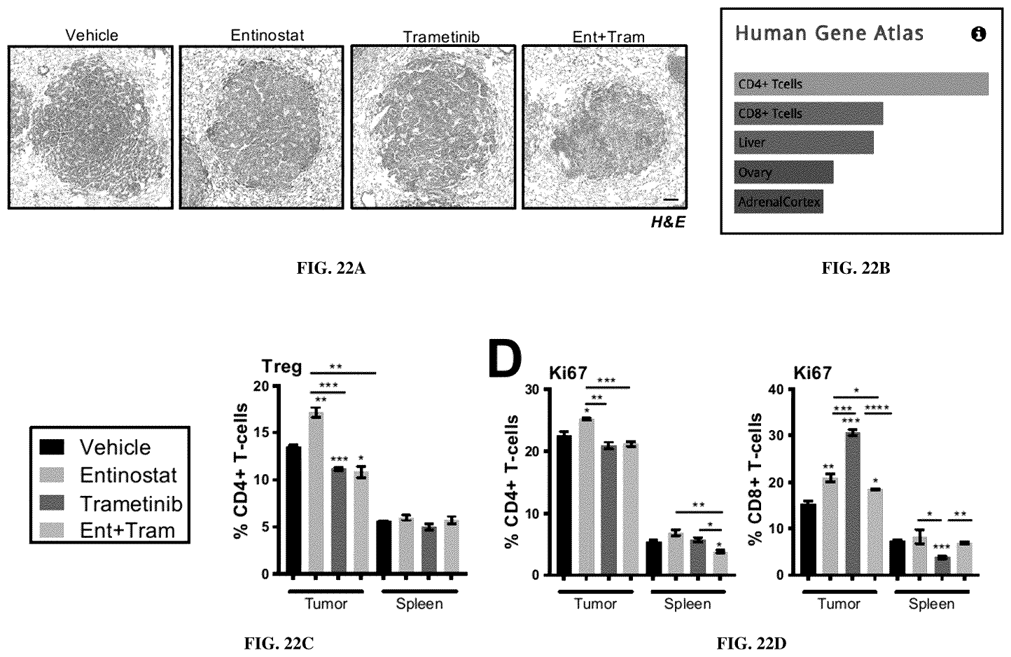

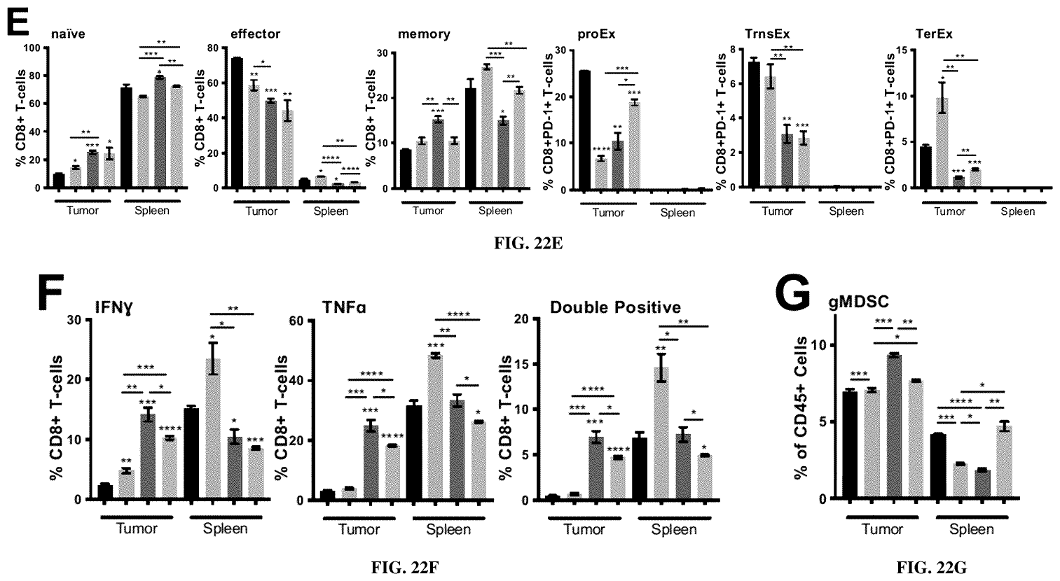

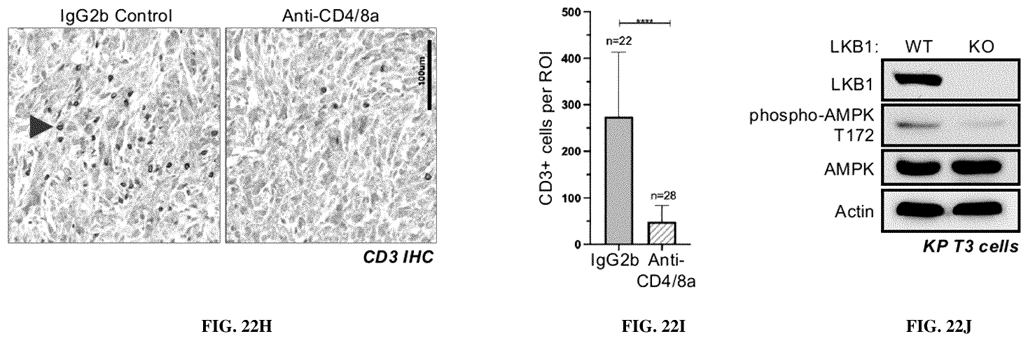

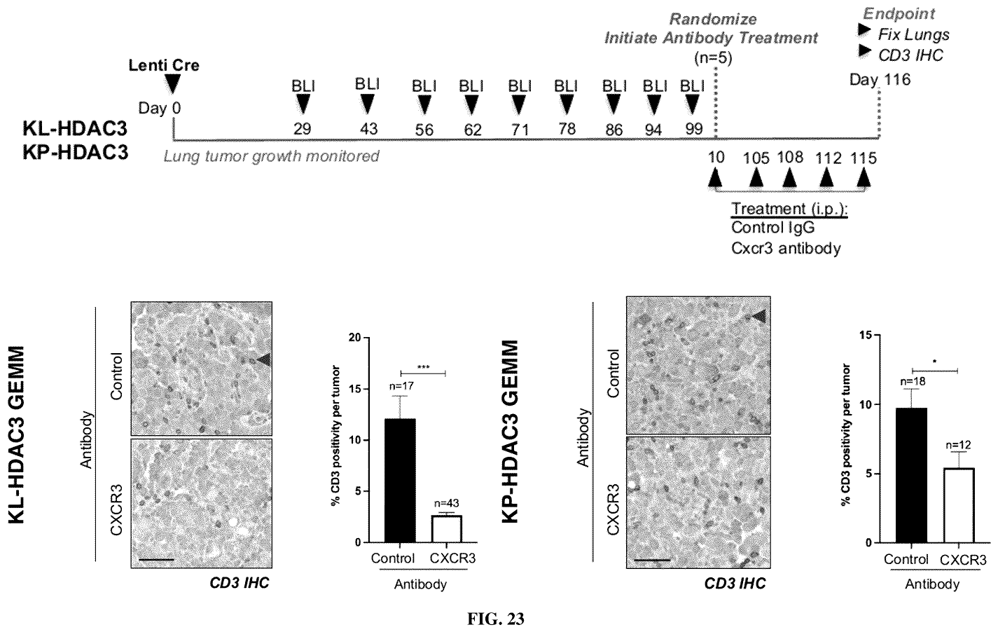

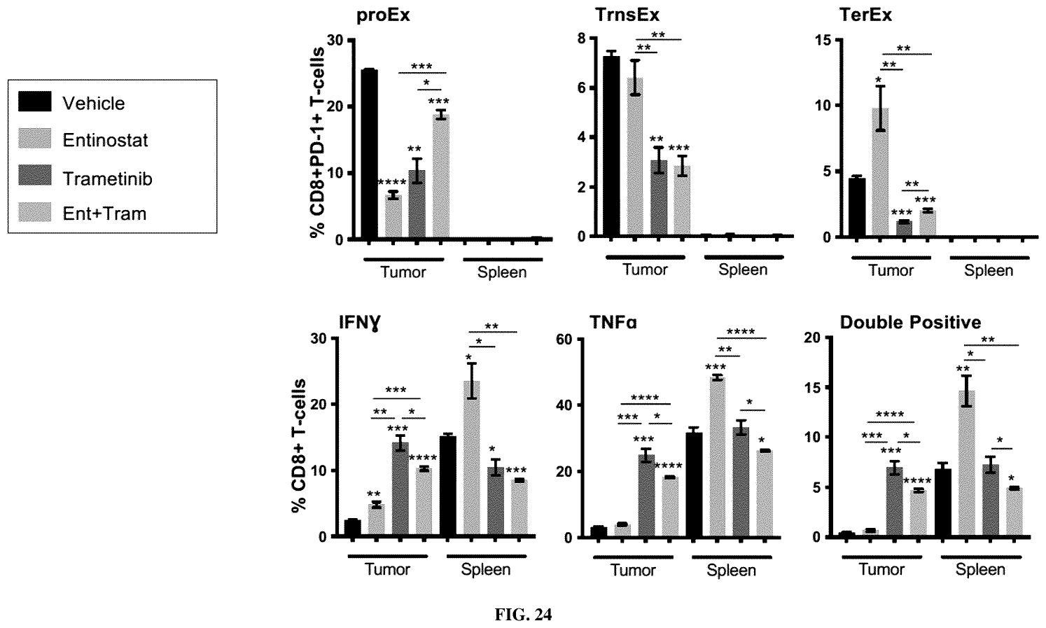

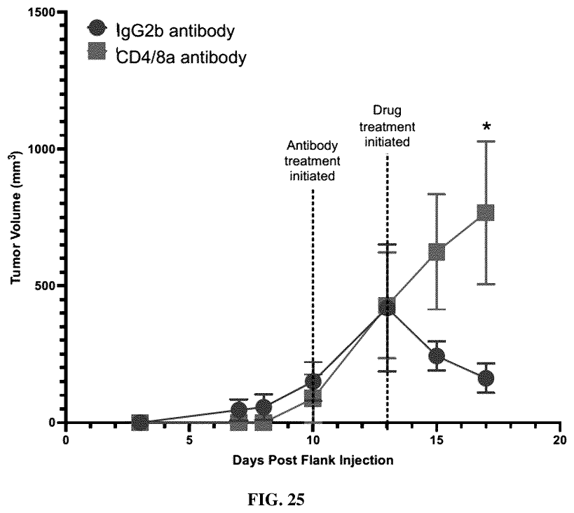

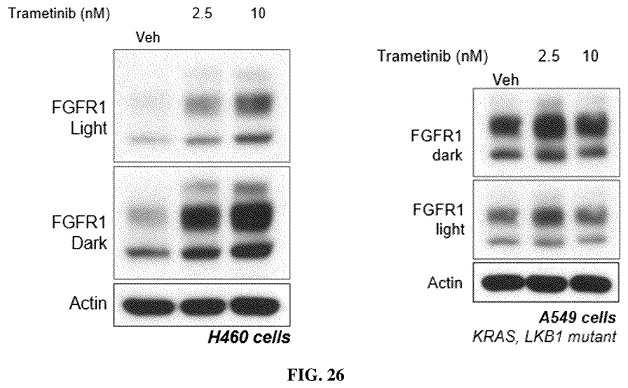

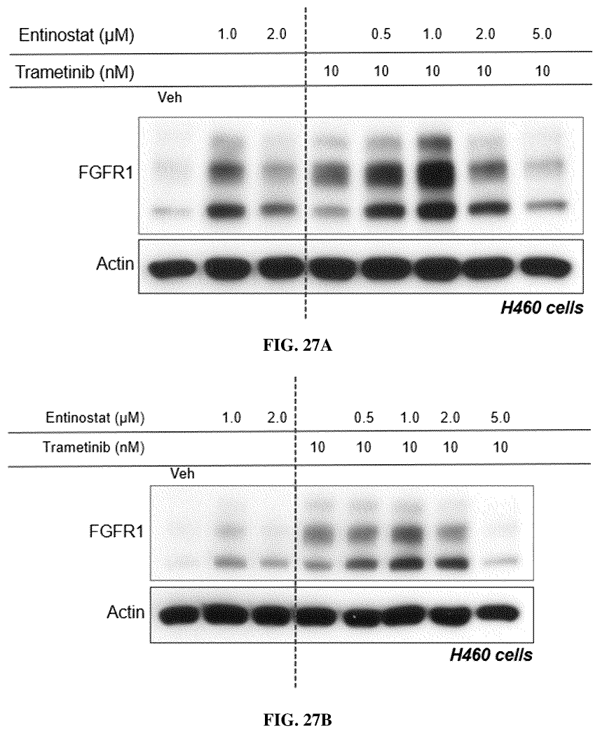

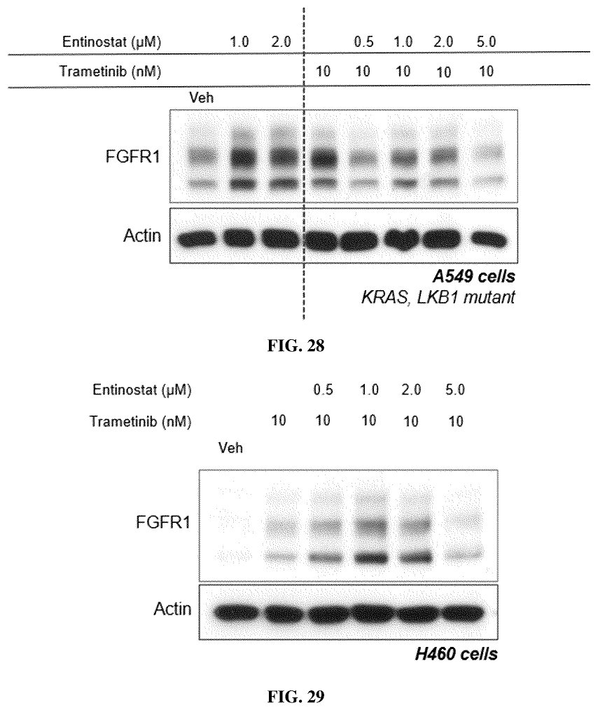

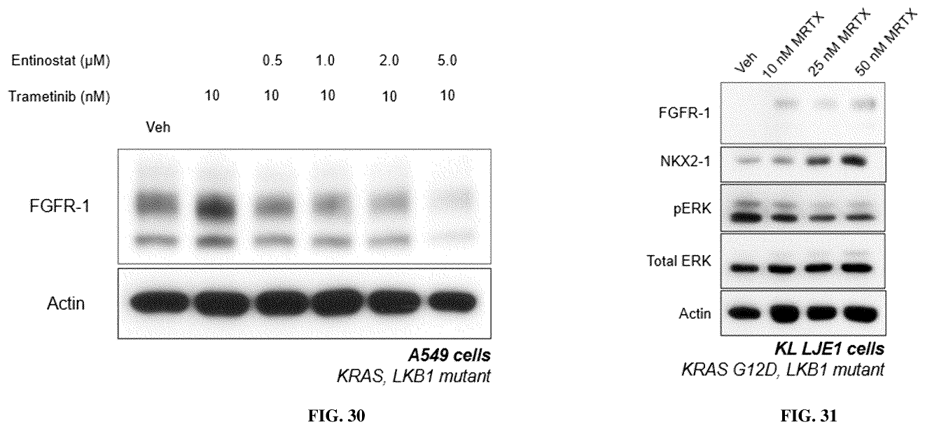

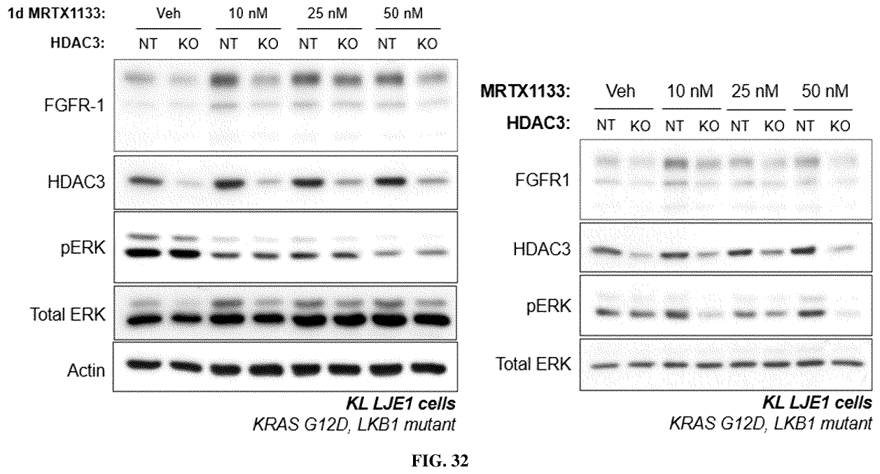

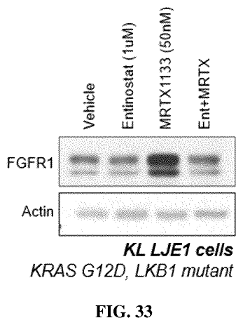

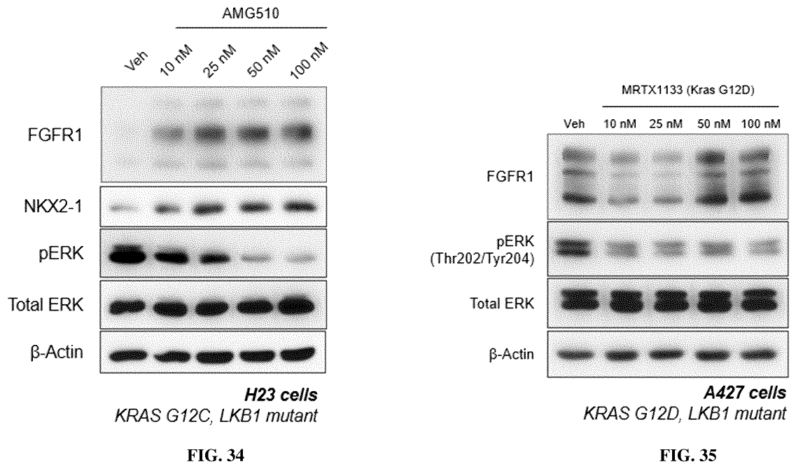

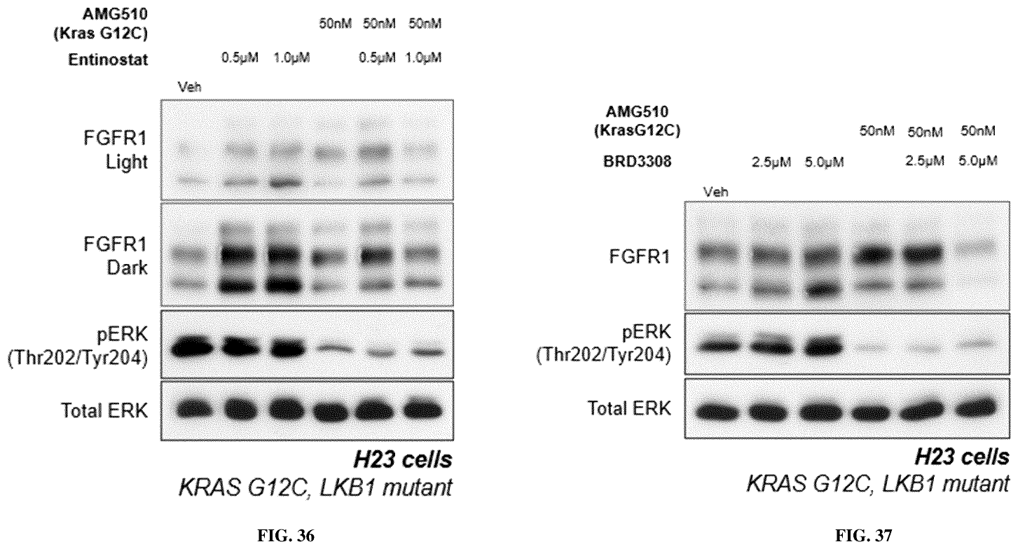

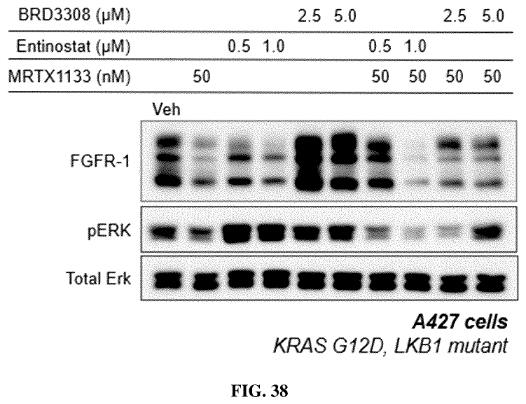

Black scale bar 20 um, red scale bar 40 um. (B) Quantitation of Cxc15 IHC (FIG. 14A) in KL or KL-HDAC3 tumors. (C) Diagram of experimental design for the 5 day in vivo entinostat treatment of the KL NSCLC GEMM. Tumors were initiated in KL mice, monitored by bioluminescence imaging biweekly until tumor burden in all mice was greater than 5x107 photons/sec. Mice were randomized, and then treated for 5 days with vehicle or entinostat (10 mg/kg/day) by oral gavage. (D) qRT-PCR on primary tumors from KL mice treated 5 days with vehicle (Veh) or 10 mg/kg/day entinostat (Ent) by oral gavage as indicated in FIG. 4C. (n=6 tumors from 3 different mice). (E) qRT-PCR on livers from KL mice (same mice as in FIG. 14C) treated 5 day with vehicle or 10 mg/kg/day entinostat. (n=4). (F) qRT-PCR on NT or HDAC3 KO KL LJE1 cells treated with Vehicle or MRTX1133 (10, 25, or 50 nM) for 5 days.

Values are expressed as mean±s.e.m. * p-value<0.05, ** p-value<0.01, *** p-value<0.001, **** p-value<0.0001 as determined by two-tailed student's t-test with Welch's correction.

FIGS. 15A-15K. HDAC3 genetic deletion in KL and KP GEMM lung tumors results in T-cell recruitment. (A) Representative H&E-stained images of KL and KL-HDAC3 tumors. Red scale bar 100 um, Black scale bar 20 um. Arrows highlight areas of tumor-infiltrating immune cells. (B) Representative images of CD3 IHC in KL and KL-HDAC3 tumors. Black scale bar 20 um. (C) Quantitation of CD3 IHC in FIG. 15B. (D) Representative H&E-stained images of KP and KP-HDAC3 tumors. Red scale bar 50 um, Black scale bar 20 um. Arrows highlight areas of tumor-infiltrating immune cells. (E) Representative images of CD3 IHC in KP and KP-HDAC3 tumors. Black scale bar 20 um. (F) Quantitation of CD3 IHC in FIG. 15E. (G) HDAC3 ChIP-seq, H3K27ac ChIP-seq, H3K9ac ChIP-seq, and Input tracks in NT and HDAC3 KO KL LJE1 cells at the Cxc110 genomic locus. (H) qRT-PCR on NT or HDAC3 KO KL LJE1 cells (n=3). (I) qRT-PCR from KL LJE1 cells treated with vehicle (Veh) or 2 uM entinostat (Ent) for 3 days (n=3). (J) qRT-PCR on NT or HDAC3 KO A549 cells (n=3). (K) qRT-PCR from A549 cells treated with vehicle (Veh) or 2 uM entinostat (Ent) for 3 days (n=3). Values are expressed as mean±s.e.m. ** p-value<0.01, *** p-value<0.001, **** p-value<0.0001 determined by two-tailed student's t-test with Welch's correction.

FIGS. 16A-16F. T-cell infiltration into GEMM lung tumors is enhanced by co-treatment with Class I HDAC inhibitor. (A) qRT-PCR from KL LJE1 cells treated with Vehicle, 1 uM entinostat (Ent), 10 nM trametinib (Tram), or the combination for 6 days (n=3). (B) Representative H&E-stained images of tumors from KL mice treated 3 weeks with vehicle, 5 mg/kg entinostat, 1 mg/kg trametinib, or entinostat plus trametinib (Ent+Tram). Scale bar 50 um. (C) Representative images of CD3 IHC on lung tumors from treated KL mice. Scale bar 50 um. (D) Quantitation of CD3 IHC in FIG. 16C. (E) Flank tumor assay with KP T3 cells in FVB mice.

Vertical dashed lines indicate initiation of treatments: Black line; start of weekly i.p. treatment with IgG2b control or anti-CD4/8a antibody. Blue line; start of daily oral gavage treatment with 5 mg/kg entinostat and 1 mg/kg trametinib. (n=6-8.) (F) Flank tumor assay with KP T3 cells with LKB1 deletion in FVB mice. Vertical dashed lines as in (E). (n=4-6.)Values are expressed as mean±s.e.m. * p-value<0.05, ** p-value<0.01, *** <0.001, **** <0.0001 determined by two-tailed student's t-test with Welch's correction.

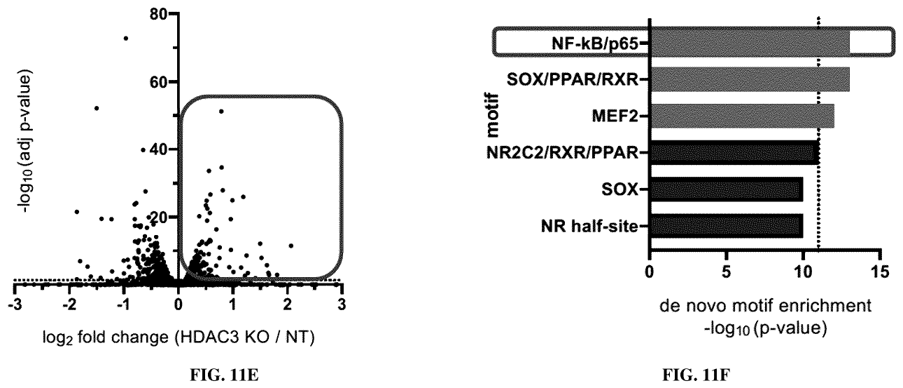

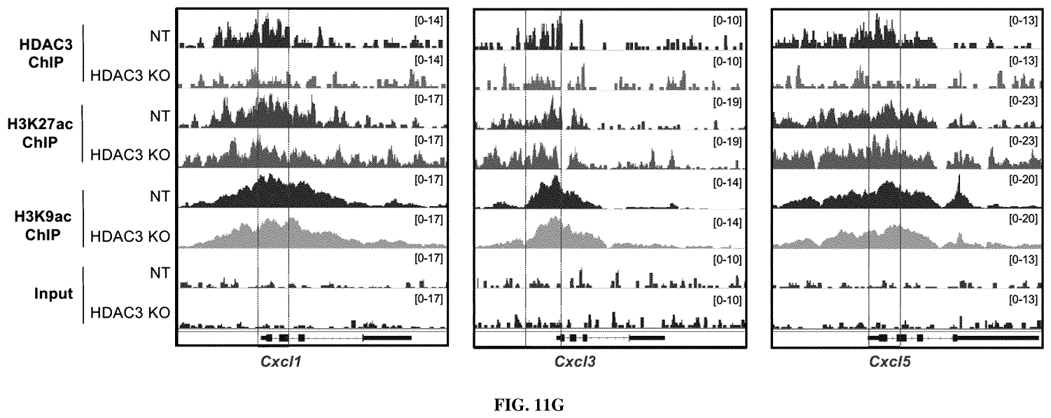

FIGS. 17A-17H. HDAC3 target genes common to KL and KP NSCLC cells are associated with p65 NF-kB and SASP pathways. (A-C) Western blot analysis of HDAC3 KO by CRISPR/Cas9 in polyclonal lysates in (A) KL LJE7 cells, (B) KP 634T cells, and (C) KP T3 cells. (D) Proliferation assessment 5 days after plating HDAC3 KO or NT cells from KL LJE7, KL LJE1, KP 634T, and KP T3 cell lines (n=6). (E) Overlap of genes downregulated upon HDAC3 KO (using all sgRNAs tested) compared to NT using RNA-seq data from KL LJE1, KL LJE7, KP T3, and KP 634T cell lines using adj. p-value<0.05 and fold change >+/−0.5 cut-offs. (F) Average HDAC3 ChIP-seq fragment depth+/−2kb of each peak center for the 3,728 HDAC3 ChIP-seq peaks identified in KL LJE1 NT cells. (G) Average H3K27ac ChIP-seq fragment depth +/−2kb of each peak center for the HDAC3 ChIP-seq peaks associated with upregulated gene expression upon HDAC3 KO (red box, FIG. 11E) in KL LJE1 cells. (H) Average H3K9ac ChIP-seq fragment depth+/−2kb of each peak center for the HDAC3 ChIP-seq peaks associated with upregulated gene expression upon HDAC3 KO (red box, FIG. 11E) in KL LJE1 cells.

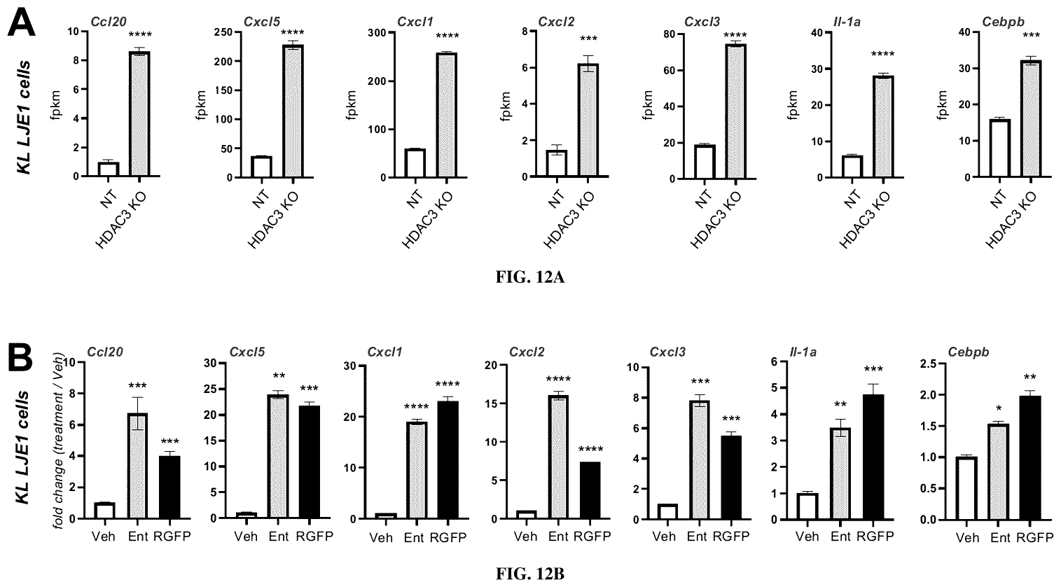

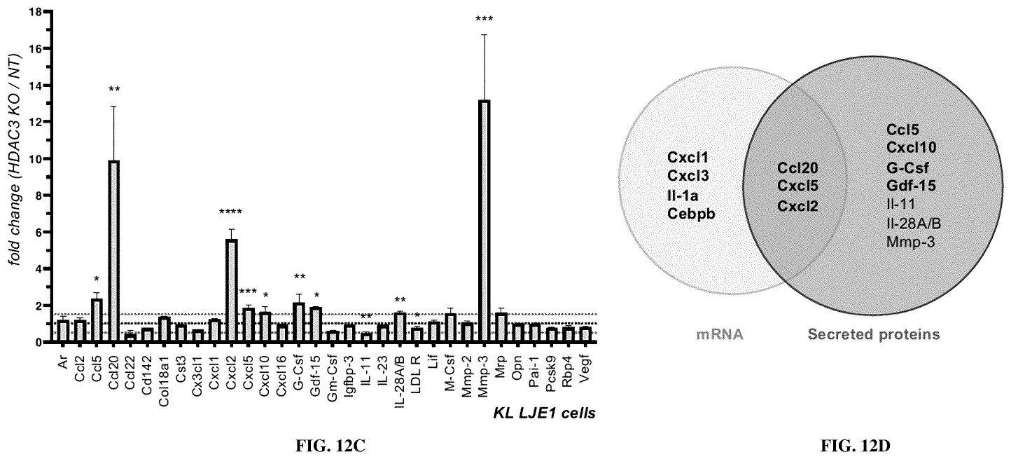

Values are expressed as mean±s.e.m. ** p-value<0.01, *** p-value<0.001, **** p-value<0.0001 determined by two-tailed student's t-test with Welch's correction FIGS. 18A-18H. HDAC3 represses chemokine gene expression in mouse and human NSCLC cells. (A) Fpkm values from RNA-seq data from KL LJE7 cells deleted for HDAC3 using CRISPR/Cas9 as in FIG. 12A (n=3). (B) Fpkm values from RNA-seq data from KP T3 cells deleted for HDAC3 using CRISPR/Cas9 (n=3). (C) Western blot (left) and qRT-PCR (right) from human A549 cells deleted for HDAC3 using CRISPR/Cas9 (n=3). (D) qRT-PCR from A549 cells treated with vehicle (Veh) or 2 uM entinostat (Ent) for 3 days (n=3). (E) qRT-PCR from H460 cells treated with vehicle (Veh) or 1 uM entinostat (Ent) for 4 days (n=3). (F) qRT-PCR from A549 cells treated with 5 uM BRD3308 (BRD) for 4 days (n=3). (G) qRT-PCR from H460 cells treated with 5 uM BRD3308 (BRD) for 4 days (n=3). (H) qRT-PCR from KL LJE1 cells deleted for HDAC3 using CRISPR/Cas9 as in FIG. 12A (n=3). Values are expressed as mean±s.e.m. * p-value<0.05, ** p-value<0.01, *** p-value<0.001, pvalue<0.0001 as determined by two-tailed student's t-test with Welch's correction.

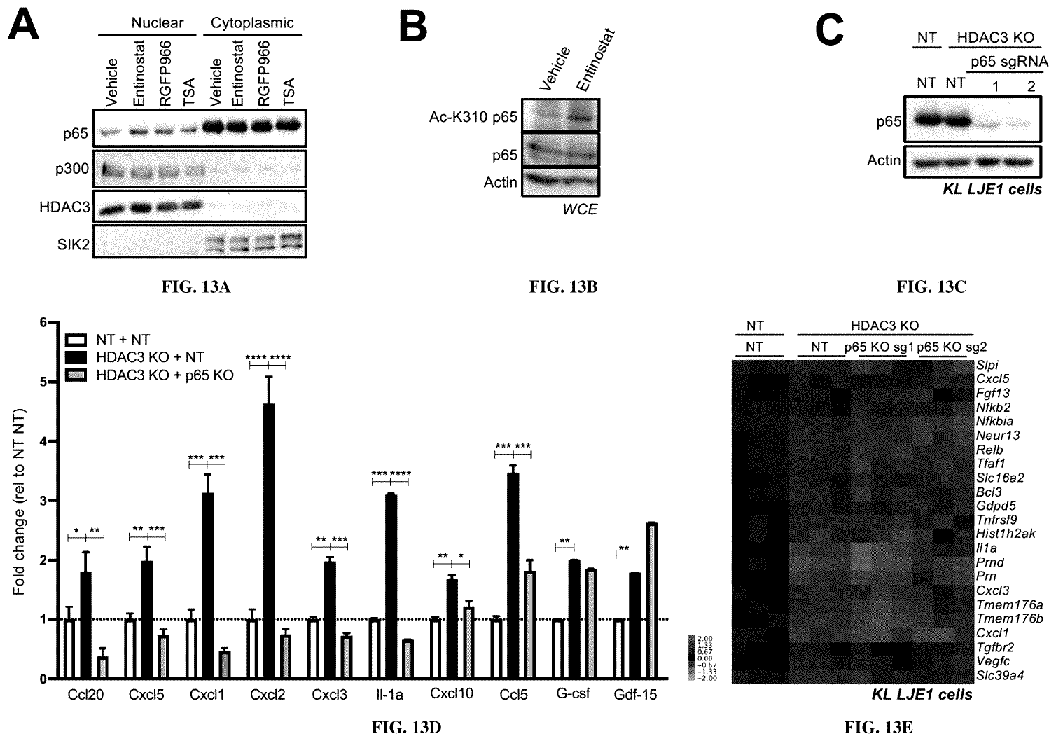

FIGS. 19A-19H. p65 NF-kB for HDAC3 repression of chemokine genes in NSCLC cells. (A) Fpkm from RNA-seq data from KL LJE1 cells deleted for p65 (KO) using CRISPR/Cas9 (n=3). g, sgRNA. NT, non-targeting.(B) Heatmap showing fpkm read counts of a gene cluster upregulated upon HDAC3 KO in a p65-dependent manner from RNA-seq data generated from KL LJE1 cells in FIG. 13A. (C) qRT-PCR on NT or p65 KO KL LJE1 cells treated with vehicle (Veh) or 1 uM entinostat (Ent) for 3 days (n=3). (D) qRT-PCR on NT or p65 KO KL LJE1 cells treated with vehicle (Veh) or 5 uM BRD3308 (BRD) for 3 days (n=3). (E) Western blot analysis of protein lysates from KL LJE1 cells transfected with GFP-p65 and treated 6 hours with 2 uM entinostat. (F) Western blot analysis of protein lysates from KL LJE1 cells expressing p65 wildtype (WT) cDNA or p65 with K310A mutation (K310A), and treated 5 hours with vehicle (Veh), 1 uM entinostat (Ent), or 5 uM BRD3308 (BRD). (G) qRT-PCR on KL LJE1 cells expressing p65 WT or p65 K310A cDNA (n=3). (H) qRT-PCR on KL LJE1 cells expressing p65 WT or p65 K310A treated 3 days with vehicle (Veh) or 1 uM entinostat (Ent) (n=3). Values are expressed as mean±s.e.m. * p-value<0.05, ** p-value<0.01, *** p-value<0.001, **** p-value<0.0001 as determined by two-tailed student's t-test with Welch's correction.

FIGS. 20A-20E. HDAC3 represses the HRCG transcriptional program in vivo in the KP and KL GEMM of NSCLC. (A) Representative images of Cxc15 IHC in KP and KP-HDAC3 tumors. Scale bar, 50 um. (B) Quantitation of Cxc15 IHC (FIG. 20A) in KP or KP-HDAC3 tumors. (C) Western blot on whole cell protein lysates from KL LJE1 cells wildtype (NT) or deleted for HDAC3 (KO) using CRISPR/Cas9 and treated for 24 hr with 10 nM, 25 nM, or 50 nM MRTX1133 or vehicle control (Veh). (D) qRT-PCR on NT or HDAC3 KO KL LJE1 cells treated with vehicle or MRTX1133 (10, 25, or 50 nM) for 5 days. (E) qRT-PCR on NT or HDAC3 KO KL LJE1 cells treated with vehicle (Veh) or trametinib (5, 10, or 25 nM) for 5 days. Values are expressed as mean±s.e.m. * p-value<0.05, ** p-value<0.01, *** p-value<0.001, **** p-value<0.0001 as determined by two-tailed student's t-test with Welch's correction.

FIGS. 21A-21D. HDAC3 impact on immune cell recruitment and CXCL10 expression. (A) Representative images of F4/80 (macrophages), NKp46 (NK cells), and Ly6g (neutrophils) IHC in KL and KL-HDAC3 tumors. Scale bar 25 um. (B) Representative images of F4/80 (macrophages), NKp46 (NK cells), and Ly6g (neutrophils) IHC in KP and KP-HDAC3 tumors.

Scale bar 25 um. (C) qRT-PCR from H460 cells treated with vehicle (Veh) or 1 uM entinostat (Ent) for 4 days (n=3). (D) qRT-PCR from H460 cells treated with vehicle (Veh) or 5 uM BRD3308 (BRD) for 4 days (n=3). Values are expressed as mean±s.e.m. * p-value<0.05, ** p-value<0.01, *** p-value<0.001, **** p-value<0.0001 as determined by two-tailed student's t-test with Welch's correction.

FIGS. 22A-22J. The impact of entinostat and trametinib treatment on the immune infiltrate in KL lung tumors. (A) Representative images of tumors from KL mice treated 3 weeks with Vehicle, 5 mg/kg entinostat, 1 mg/kg trametinib, or entinostat plus trametinib (Ent+Tram).

Scale bar 100 um. (B) Enrichr Cell Type analysis of genes uniquely upregulated in Ent+Tram treated tumors in RNA-seq data from individual dissected primary KL tumors isolated from treated mice. (C-F) Flow cytometry assessment of primary tumors and spleen isolated from KL mice after 6 weeks of treatment with vehicle, 10 mg/kg entinostat, 1 mg/kg trametinib, or entinostat plus trametinib (Ent+Tram). (C) FoxP3+Regulatory T-cells (Tregs) plotted as a percentage of total CD4+ cells. (D) T-cell proliferation rates. (E) Phenotypic analysis of T-cell populations. Analysis of the CD8+ T-cell compartment with the proportion of total cells that are naive (CD62L+CD44-), effector (CD62L-CD44+), or memory (CD62L+CD44+) plotted.

Characterization of T-cell exhaustion subsets: cells were gated on PD-1+CD8 Cells and were plotted as a percentage of total CD8+ cells for progenitor (proEx; PD-1+SlamF6+Tim3-), Transitory (TrnsEx; PD-1+Tim3+CD101-) and Terminally Exhausted (TerEx; PD-1+Tim3+CD101+). (F) Single cells isolated from tumors where stimulated ex-vivo with PMA and Ionomycin for four hours in the presence of Brefelden A and cytokine production was assessed by intracellular staining. (G) gMDSCs (CD45+CD11b+Ly6G+) plotted as a percentage of total CD45+ cells. (H) Representative images of CD3 IHC on flank tumors from treated mice at endpoint (FIG. 16E, day 17). Scale bar, 100 um. (I) Quantitation of CD3 IHC on flank tumors from treated mice (FIG. 22H). (J) Western blot on whole cell protein lysates from KP T3 cells inactivated for LKB1 using CRISPR/Cas9.Values are expressed as mean±s.e.m. * p-value<0.05, ** p-value<0.01, *** <0.001, **** <0.0001 determined by two-tailed student's t-test with Welch's correction.

FIG. 23. Cxc110 mediates T-cell infiltration into HDAC3-deleted Kras mutant GEMM lung tumors. (Top) Schematic of experiment testing the impact of anti-CXCR3 antibody treatment in KL-HDAC3 and KP-HDAC3 GEMMs. (Bottom, left) Representative images and quantitation of CD3 IHC in KL-HDAC3 tumors from mice treated as outlined above. Scale bar 50 um. (Bottom, right) Representative images and quantitation of CD3 IHC in KP-HDAC3 tumors from mice treated as outlined above. Scale bar 50 um. Values are expressed as mean±s.e.m. * p-value<0.05, ** p-value<0.01, *** p-value<0.001, **** p-value<0.0001 as determined by two-tailed student's t-test with Welch's correction.

FIG. 24. Impact of combination therapy approach on tumor immune infiltrate.

FIG. 25. A flank tumor assay using KP T3 cells showing T-cells contribute to tumor growth control in combination treated mice. Antibody treatment (i.p.): IgG2b or CD4/8a antibody. Drug treatment (oral gavage): trametinib, entinostat. n=3-4 mice.

FIG. 26. MEK inhibitor trametinib induces the FGFR1 resistance mechanism in human LKB1 mutant lung cancer cells following 13 days of treatment.

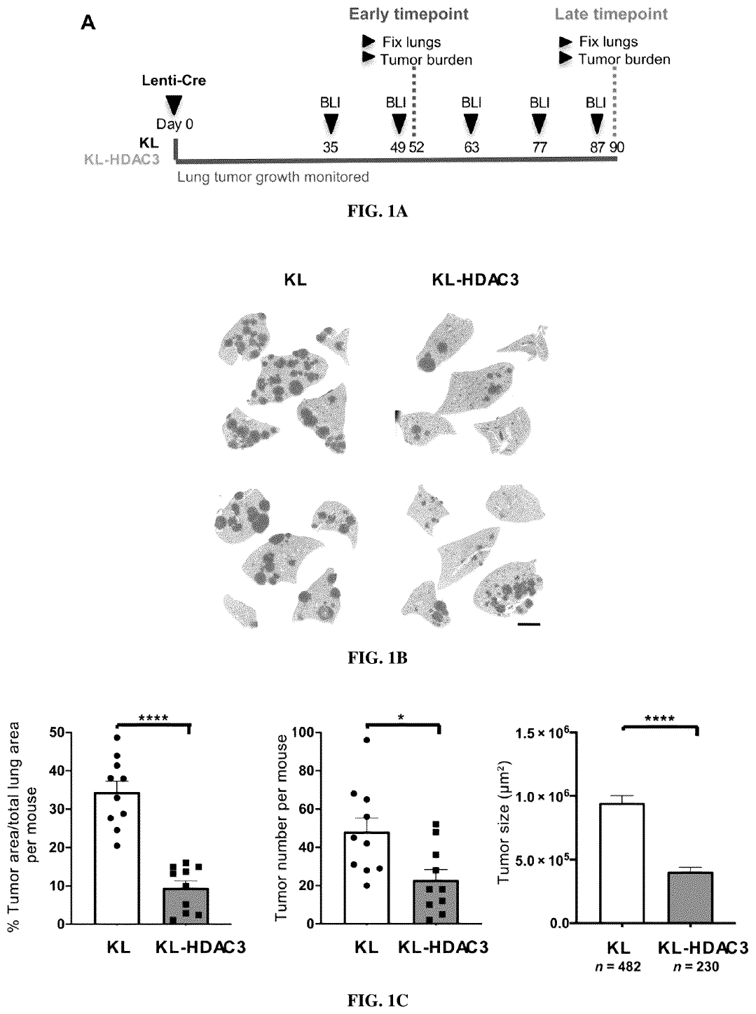

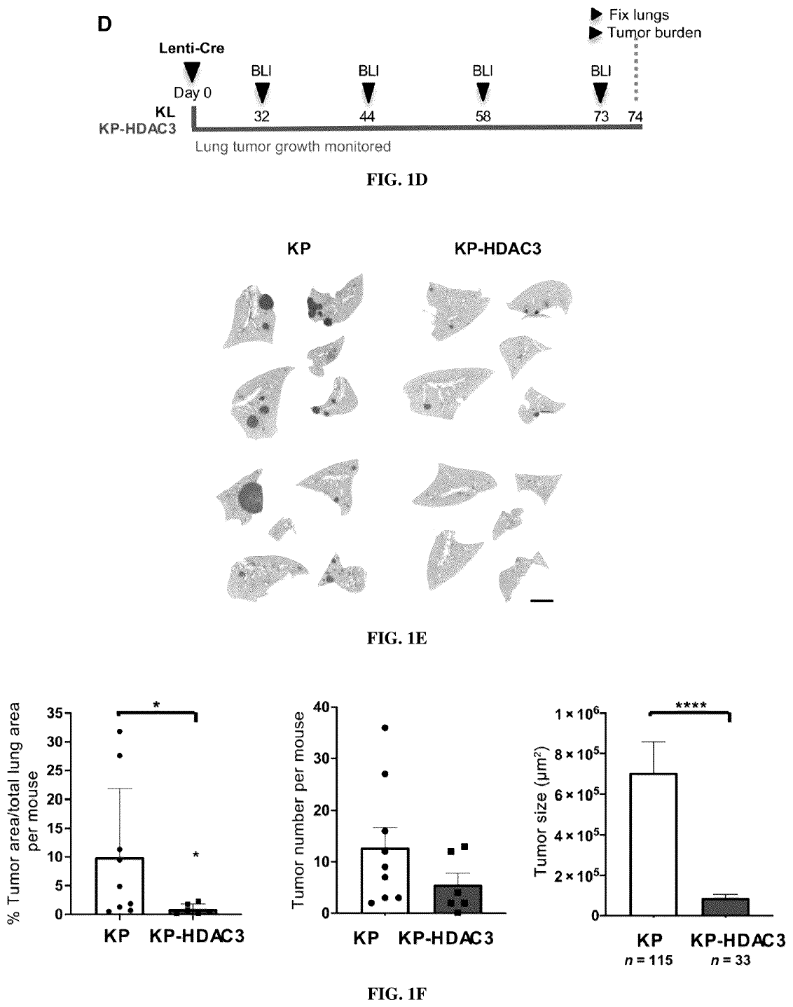

FIGS. 27A and 27B. MEK inhibitor trametinib cooperates with the HDAC1/3 inhibitor entinostat to coordinately restrain the FGFR1 resistance mechanism in human LKB1-mutant lung cancer cells following 6 days of treatment (FIG. 27A) and 1Idays of treatment (FIG. 27B).

FIG. 28. MEK inhibitor trametinib cooperates with the HDAC1/3 inhibitor entinostat to coordinately restrain the FGFR1 resistance mechanism in human KRAS, LKB1 mutant lung cancer cells following 6 days of treatment.

FIG. 29. HDAC1/3 inhibitor entinostat reverses the impact of Trametinib on the FGFR1 resistance mechanism in human LKB1-mutant lung cancer cells following 9 days of trametinib treatment, entinostat treatment days 3-9 (6 days total).

FIG. 30. HDAC1/3 inhibitor entinostat reverses the impact of Trametinib on the FGFR1 resistance mechanism in human KRAS, LKB1-mutant lung cancer cells following 9 days of trametinib treatment, entinostat treatment days 3-9 (6 days total).

FIG. 31. KRAS G12D inhibitor MRTX1133 induces the FGFR1 resistance mechanism in KL LJE1 mouse lung cancer cells following 5 days of treatment.

FIG. 32. KRAS G12D inhibitor MRTX1133 induces the FGFR1 resistance mechanism in an HDAC3-dependent manner in KL LJE1 mouse lung cancer cells following 1 day of treatment (left) and 5 days of treatment (right).

FIG. 33. KRAS inhibitors cooperate with the HDAC1/3 inhibitor entinostat to coordinately restrain the FGFR1 resistance mechanism in mouse KRAS, LKB1 mutant lung cancer cells following 13 days of treatment.

FIG. 34. KRAS G12C inhibitor AMG510 induces the FGFR1 resistance mechanism in H23 human lung cancer cells following 6 days of treatment.

FIG. 35. KRAS G12D inhibitor MRTX1133 induces the FGFR1 resistance mechanism in human lung cancer cells following 6 days of treatment.

FIG. 36. KRAS G12C inhibitor AMG510 cooperates with the HDAC1/3 inhibitor entinostat to coordinately restrain the FGFR1 resistance mechanism in human lung cancer cells following 6 days of treatment.

FIG. 37. KRAS G12C inhibitor AMG510 cooperates with the HDAC3 inhibitor BRD3308 to coordinately restrain the FGFR1 resistance mechanism in human lung cancer cells following 6 days of treatment.

FIG. 38. KRAS G12D inhibitor MRTX1133 cooperates with the HDAC1/3 inhibitor entinostat and HDAC3 inhibitor BRD3308 to coordinately restrain the FGFR1 resistance mechanism in human lung cancer cells following 13 days of treatment.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a” “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method.

Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating,” and the like refer to obtaining a desired pharmacologic and/or physiologic effect against a particular disease, disorder, or condition.

Preferably, the effect is therapeutic, i.e., the effect partially or completely cures the disease and/or adverse symptom attributable to the disease.

The term “pharmaceutical formulation” as used herein refers to a composition comprising at least one pharmaceutically-active agent, chemical substance or drug. The pharmaceutical formulation may be in solid or liquid form and can comprise at least one additional active agent, carrier, vehicle, excipient or auxiliary agent identifiable by the skilled person. The pharmaceutical formulation may be in the form of a tablet, capsule, granules, powder, liquid or syrup.

The term “effective dose” or “effective amount” refers to an amount of an agent, e.g., a neutralizing antibody, that results in the reduction of symptoms in a patient, treatment of prevention of a disease or condition, or results in a desired biological outcome.

As used herein, the terms “administration” and “administering” refer to the act of giving a drug, prodrug, or other agent, or therapeutic to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Provided herein are pharmaceutical compositions comprising KRAS inhibitors and HDAC inhibitors and methods of administering KRAS inhibitors and HDAC inhibitors for the treatment/prevention of cancer. In particular, an HDAC inhibitor is administered to overcome KRAS inhibitor resistance in KRAS, LKB1 mutant lung cancer.

While HDAC inhibitors are FDA-approved in hematopoietic malignancies, their efficacy in solid tumors has been wanting, which has been conjectured to be due to limiting toxicity from current agents inhibiting multiple HDAC family members, as well as lack of insights into the optimal clinical contexts where HDAC inhibitors may synergize with other currently approved or in-development therapeutics (Ref. 54; incorporated by reference in its entirety). Very little genetic analysis of individual Class I HDACs in genetic engineered mouse models has been performed, despite extensive data that HDAC1/2 form completely distinct protein complexes with completely distinct downstream targets from HDAC3 or HDAC8. HDAC3 is infrequently directly mutated nor is its expression frequently altered in cancer, which has contributed to HDAC3 function in cancer being overlooked despite its appreciated importance in normal tissue homeostasis. Notably however, recent studies have identified HDAC3 as playing a specific role in lymphomas (Refs. 55-57; incorporated by reference in their entireties), rhabdomyosarcoma (Ref. 58; incorporated by reference in its entirety), melanomas (Ref. 59; incorporated by reference in its entirety), and pancreatic cancer (Ref. 60; incorporated by reference in its entirety) settings. Genetic deletion of HDAC3 in normal tissues in vivo has not characterized HDAC3 as a ubiquitous regulator of cell growth or proliferation, instead identifying a diverse range of tissue specific functions (Ref. 19; incorporated by reference in its entirety). In liver, HDAC3 loss was associated with severe metabolic derangements after 5 months and, subsequently, hepatocellular carcinoma formation was observed 10 months after deletion (Ref. 61; incorporated by reference in its entirety), indicating that HDAC3 is not only not required for growth of all tumors as we observe here in lung, but indeed in liver loss of HDAC3 leads to more, not less, tumor burden.

And yet, mutation of the bonified but poorly characterized HDAC3 protein complex member TBL1XR1 has been proven to drive immunoblastic lymphoma in vivo by aberrantly redirecting HDAC3 complex function (Ref. 55; incorporated by reference in its entirety). CREBBP, which is frequently mutated in B-cell lymphoma, was also reported to drive in vivo tumorigenesis via HDAC3 (Refs. 56-57; incorporated by reference in their entireties).

Experiments conducted during development of embodiments herein demonstrate that a large fraction of HDAC3 target genes in NSCLC tumors in vivo are also dependent on the lineage specific transcription factor NKX2-1. NKX2-1 is a member of the NKX sub-class of homeobox proteins, which is a large class of transcription factors that share a related DNA binding domain, the homeodomain. Much of the understanding of the NKX subclass of homeodomain proteins, which contain a tyrosine at position 54 not present in other homeodomains, originated from developmental biology studies which identified striking mutant phenotypes. For example, studies of tinman, the Drosophila homologue of murine Nkx2-5, identified that it is a critical regulator of mesodermal cell fate required for cardiac development (Ref. 62; incorporated by reference in its entirety). Cloning of vertebrate Nkx2-1 (77F-1, Titf1, T/ebp; vnd in Drosophila) facilitated the identification of its selective expression and activity in thyroid, lung, and restricted areas of the brain (Refs. 63-64; incorporated by reference in their entireties), and the timing of its expression at the onset of organ development immediately implicated NKX2-1 as a lineage determining factor (Refs. 64-65; incorporated by reference in their entireties). Subsequent studies confirmed the requirement for NKX2-1 in proper lung and thyroid development, and NKX2-1 dysfunction is clearly associated with human disease in these tissues (Refs. 41, 66; incorporated by reference in its entirety). In lung adenocarcinoma (LUAD) NKX2-1 is considered a lineage-specific oncogene when overexpressed (in ˜80% of cases) or amplified (in up to 15% of cases), and notably NKX2-1 is the most amplified gene in human LUAD (26, 40-43). Yet NKX2-1 expression has also been reported to associate with favorable prognosis in early stage LUAD (Ref. 67; incorporated by reference in its entirety), and in the Kras and KP lung cancer models, NKX2-1 suppresses tumor growth, enforces a lineage-specific differentiation program (Ref. 44; incorporated by reference in its entirety), and restrains metastatic potential (Ref. 46; incorporated by reference in its entirety). In contrast, in an EGFR-driven NSCLC GEMM model NKX2-1 inactivation suppressed lung tumorigenesis (Ref. 68; incorporated by reference in its entirety), highlighting the duality of NKX2-1 with respect to both tumor promoting or tumor suppressing function. The molecular determinants that drive these divergent functions remain to be fully identified. In early-stage LUAD oncogenic KRAS leads to loss of lineage identity in alveolar epithelial progenitor (AT2) cells associated with reduced NKX2-1 transcriptional output (Ref. 69; incorporated by reference in its entirety).

NKX2-1 expression can be impacted by multiple upstream TFs, and its transcriptional activity can be modulated in a context-dependent manner by post-translational modification and/or cooperation with additional TFs and cofactors (Ref. 41; incorporated by reference in its entirety), much of which remains to be comparatively elucidated between NSCLC subtypes. Experiments conducted during development of embodiments herein identified selective cooperation between NKX2-1 and HDAC3 on a specific set of target genes in LKB1-mutant cells. Mucinous dedifferentiation upon HDAC3 deletion in KL or KP tumors in vivo was not observed, indicating that HDAC3 inactivation is not equivalent to complete NKX2-1 deletion in vivo (Ref. 44; incorporated by reference in its entirety), but that HDAC3 functions instead as a coregulator of NKX2-1.

Beyond basal gene expression regulation, experiments conducted during development of embodiments herein demonstrate that the activity of the HDAC3/NKX2-1 complex is induced in KL NSCLC cells as a resistance mechanism to MEK inhibitors, an effect reversed by HDAC3 inhibition. Multiple existing targeted therapies induce clinically well-documented acquired resistance, and efforts to identify molecular resistance mechanisms have become major lines of investigation. The finding that the LUAD lineage transcription factor NKX2-1 is hyperactivated by trametinib resistance indicates a connection between resistance mechanisms and lineage identity.

Experiments conducted during development of embodiments herein have revealed a specific therapeutic context, trametinib resistance, where HDAC3 inhibition has utility in NSCLC.

Experiments conducted during development of embodiments herein have revealed HDAC3 actively restrains T-cell infiltration into lung tumors via a tumor cell-intrinsic mechanism, and that disruption of HDAC3-mediated repression, by genetic deletion or pharmacological approaches, resulted in T-cell influx into lung tumors. This finding can be leveraged to enhance recruitment of functional T-cells into GEMM lung tumors in vivo this finding can be leveraged to enhance recruitment of functional T-cells into GEMM lung tumors in vivo via systemic administration of clinically-tolerated therapeutics. HDAC3-targeting therapeutics may have the capacity to elicit T-cell infiltration into NSCLC tumors independently of mechanisms employed by immune checkpoint inhibitors (ICIs). Clinically, KRAS, LKB1 mutant lung cancer is resistant to ICIs, and these tumors are characterized by low PD-L1 expression and a paucity of tumor-infiltrating T-cells.

In some embodiments, compositions and methods herein comprise a HDAC inhibitor. In some embodiments, the HDAC inhibitor is a general HDAC inhibitor. In some embodiments, the HDAC inhibitor inhibits HDAC3. In some embodiments, the HDAC inhibitor is specific for HDAC3.

Examples of histone deacetylase inhibitors that find use in certain embodiments herein include hydroxamic acids (or hydroxamates) such as trichostatin A, cyclic tetrapeptides (such as trapoxin B) and depsipeptides, benzamides, electrophilic ketones, aliphatic acid compounds such as phenylbutyrate and valproic acid, hydroxamic acids such as vorinostat (SAHA), belinostat (PXD101), LAQ824, and panobinostat (LBH589), benzamides such as entinostat (MS-275), CI994, and mocetinostat (MGCD0103), nicotinamide, derivatives of NAD, dihydrocoumarin, naphthopyranone, and 2-hydroxynaphaldehydes. Exemplary HDAC inhibitors include, but are not limited to, vorinostat (SAHA); entinostat (MS-275); panobinostat (LBH589); TSA (Trichostatin A); romidepsin; mocetinostat (MGCD0103); RGFP966; belinostat; tubastatin A; ricolinostat (ACY-1215); quisinostat (JNJ-26481585) 2HCl; MC1568; tubastatin A HCl; PCI-34051; curcumin; tacedinaline (CI994); LMK-235; fimepinostat (CUDC-907); tubacin; Givinostat (ITF2357); Valproic Acid sodium; VPA (Valproic acid); AR-42; TMP269; Sodium butyrate; pracinostat (SB939); CUDC-101; abexinostat (PCI-24781); santacruzamate A (CAY10683); (−)-parthenolide; dacinostat (LAQ824); CAY10603; RG2833 (RGFP109); tasquinimod; 4-PBA (Sodium Phenylbutyrate); tucidinostat (Chidamide); TMP195; nexturastat A; sulforaphane; droxinostat; domatinostat (4SC-202); scriptaid; 4-PBA (4-Phenylbutyric acid); M344; resminostat; citarinostat (ACY-241); BG45; WT161; ACY-738; HPOB; tubastatin A TFA; TC-H 106; SIS17; divalproex Sodium; ACY-775; BML-210 (CAY10433); TH34; raddeanin A; sinapinic Acid; KT-531; ITF3756; tefinostat(CHR-2845); pyroxamide (NSC 696085); UF010; SKLB-23bb; isoguanosine; NKL 22; BRD73954; CXD101; suberohydroxamic acid; BRD3308; splitomicin; biphenyl-4-sulfonyl chloride; KA2507; SR-4370; GSK3117391; tinostamustine(EDO-S101); and bocodepsin (OKI-179).

In some embodiments, a HDAC inhibitor for use in the compositions and methods herein is a HDAC3 inhibitor. In some of these embodiments, the HDAC3 inhibitor is trichostatin A, vorinostat (Proc. Natl. Acad. Sci. U.S.A. 1998 Mar. 17; 95(6):3003-7; incorporated by reference in its entirety), givinostat, abexinostat (Mol. Cancer Ther. 2006 May;5(5): 1309-17; incorporated by reference in its entirety), belinostat (Mol. Cancer Ther. 2003 August; 2(8):721-8; incorporated by reference in its entirety), panobinostat (Clin. Cancer Res. 2006 Aug. 1; 12(15):4628-35; incorporated by reference in its entirety), resminostat (Clin. Cancer Res. 2013 Oct. 1; 19(19):5494-504; incorporated by reference in its entirety), quisinostat (Clin. Cancer Res. 2013 Aug. 1; 19(15):4262-72; incorporated by reference in its entirety), depsipeptide (Blood. 2001 Nov. 1; 98(9):2865-8; incorporated by reference in its entirety), entinostat (Proc. Natl. Acad. Sci. U.S.A. 1999 Apr. 13; 96(8):4592-7; incorporated by reference in its entirety), mocetinostat (Bioorg. Med. Chem. Lett. 2008 Feb. 1; 18(3):1067-71; incorporated by reference in its entirety) or valproic acid (EMBO J. 2001 Dec. 17; 20(24):6969-78; incorporated by reference in its entirety).

In some embodiments, an HDAC inhibitor that is capable of inhibiting HDAC3 is provided. Examples of HDAC3 inhibitors that find use in embodiments herein include, but are not limited to:

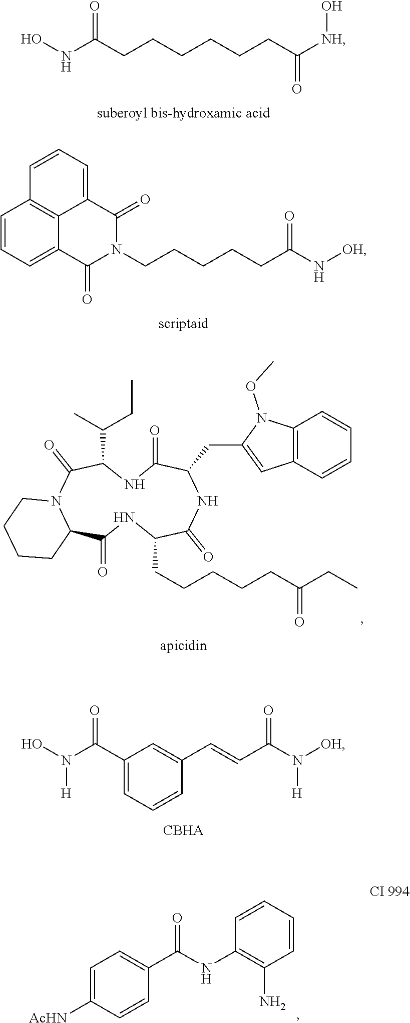 [SMILES: O=C(CCCCCCC(=O)NO)NO ; O=C(CCCCCN1C(=O)c2cccc3cccc(c23)C1=O)NO ; CCC(=O)CCCCC[C@@H]1NC(=O)[C@H]2CCCCN2C(=O)[C@H](C(C)CC)NC(=O)[C@H](Cc2cc3ccccc3n2OC)NC1=O ; O=C(/C=C/c1cccc(C(=O)NO)c1)NO ; CC(=O)Nc1ccc(C(=O)Nc2ccccc2N)cc1]

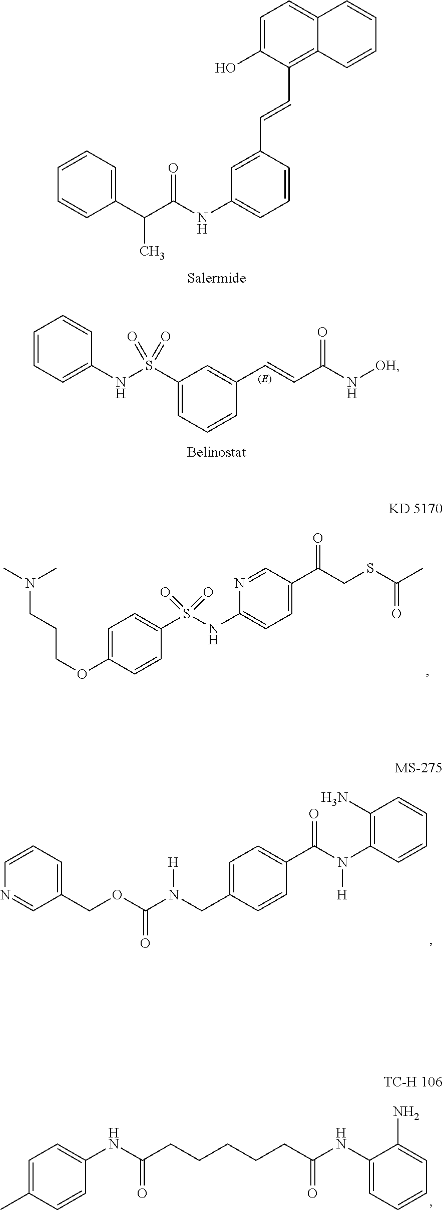

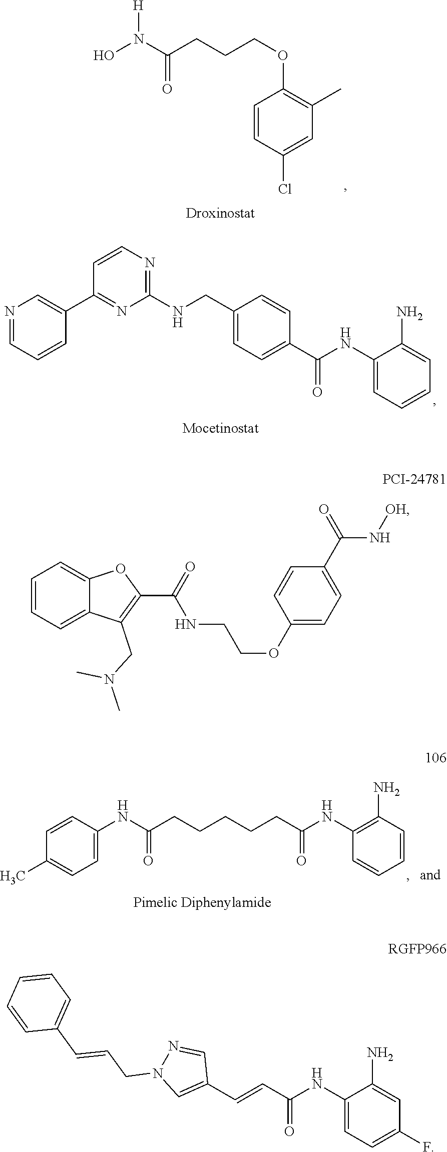 [SMILES: Cc1cc(Cl)ccc1OCCCC(=O)NO ; Nc1ccccc1NC(=O)c1ccc(CNc2nccc(-c3cccnc3)n2)cc1 ; CN(C)Cc1c(C(=O)NCCOc2ccc(C(=O)NO)cc2)oc2ccccc12 ; Cc1ccc(NC(=O)CCCCCC(=O)Nc2ccccc2N)cc1 ; Nc1cc(F)ccc1NC(=O)/C=C/c1cnn(C/C=C/c2ccccc2)c1]

In some embodiments, an HDAC inhibitor (e.g., HDAC3 inhibitor) is an antibody or antibody fragment that binds to a HDAC (e.g., HDAC3) and inhibits its activity.

In some embodiments, compositions and methods herein comprise a KRAS inhibitor.

KRAS inhibitors include those inhibitors which inhibit KRAS directly and those which inhibit the KRAS pathway, e.g., signaling through the Mitogen-Activated Protein Kinase (MAPK) cascade. For example such KRAS inhibitors, include those with inhibit downstream members of the KRAS pathway, including, but not limited to, MEK, RAF, ERK. Thus, KRAS inhibitors encompass MEK inhibitors, RAF inhibitors and ERK inhibitors. Additionally, KRAS inhibitors include those inhibitors of pathogenic mutant forms of KRAS, such as KRAS G12D and G12C.

Examples of KRAS inhibitors that find use in embodiments herein include Sotorasib (AMG510), Adagrasib/MRTX849, AMG 404, trametinib, RMC-4630, afatinib, pembro, panitumumab, carbo/pem/docetaxel, everolimus, Palbociclib, bevacizumab, LY3537982, abemaciclib, erlotinib, sintilimab, temuterkib, LY3295668, cetuximab, JNJ-74699157 (ARS-3248), GDC-6036, atezo, spartalizumab, TNO155, EGF816 (nazertinib/EGFR TKI, RMC-4630, cobimetinib/Osimertinib, BI 1701963, MRTX1133 (KRAS G12D inhibitor), AMG510 (KRAS G12C inhibitor), binimetinib, cobimetinib or XL518, selumetinib, and irinotecan. In some embodiments, the KRAS inhibitor is Trametinib.

Certain embodiments herein are directed to administration of a KRAS inhibitor and an HDAC (e.g., HDAC3) inhibitor to a subject with cancer, in remission from cancer, or at elevated risk of cancer. In some embodiments, the inhibitors described herein are administered as part of therapeutic or prophylactic regimen for the treatment or prevention of acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer.

In some embodiments, the combination of a KRAS inhibitor and HDAC inhibitor (e.g., HDAC3 inhibitor) is administered for the treatment or prevention of a lung cancer (e.g., non-small cell lung cancer (NSCLC), small cell lung cancer, etc.). In some embodiments, a KRAS inhibitor and a HDAC inhibitor (e.g., HDAC3 inhibitor) are administered for the treatment or prevention of a KRAS inhibitor resistant lung cancer (e.g., trametinib resistant), such as a KRAS, LKB1 mutant lung cancer.

In some embodiments, a KRAS inhibitor and a HDAC inhibitor (e.g., HDAC3 inhibitor) are co-administered with one or more additional agents for the treatment or prevention of cancer. In some embodiments, a KRAS inhibitor and a HDAC inhibitor (e.g., HDAC3 inhibitor) are co-administered (e.g., before, during, and/or after administration of the co-therapy) with one or more cancer therapies to prevent/treat/reduce/eliminate cancer cells and/or to enhance cancer treatment.

In some embodiments, a KRAS inhibitor and a HDAC inhibitor (e.g., HDAC3 inhibitor) are co-administered with one or more chemotherapeutics. Many chemotherapeutics are presently known in the art and can be used in combination with VS. In some embodiments, the chemotherapeutic is selected from the group consisting of mitotic inhibitors, alkylating agents, anti-metabolites, intercalating antibiotics, growth factor inhibitors, cell cycle inhibitors, enzyme inhibitors, topoisomerase inhibitors, protein-protein interaction inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.RTM.; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Embodiments herein further relate to methods for using a KRAS inhibitor and HDAC (e.g., HDAC3) inhibitor in combination with radiation therapy for inhibiting abnormal cell growth or treating a hyperproliferative disorder in the mammal. Techniques for administering radiation therapy are known in the art, and these techniques can be used in the combination therapy described herein. Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

In some embodiments, a KRAS inhibitor and a HDAC inhibitor (e.g., HDAC3 inhibitor) are co-administered with one or more immunotherapies. Immunotherapies include chimeric antigen receptor (CAR) T-cell or T-cell transfer therapies, cytokine therapy, immunomodulators, cancer vaccines, or administration of antibodies (e.g., monoclonal antibodies). In some embodiments, the immunotherapy comprises administration of antibodies. The antibodies may target antigens either specifically expressed by tumor cells or antigens shared with normal cells.

In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like). In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

A KRAS inhibitor and HDAC (e.g., HDAC3) inhibitor may also be used in combination with an amount of one or more substances selected from anti-angiogenesis agents, signal transduction inhibitors, antiproliferative agents, glycolysis inhibitors, or autophagy inhibitors.

Anti-angiogenesis agents, such as MMP-2 (matrix-metalloproteinase 2) inhibitors, MMP-9 (matrix-metalloprotienase 9) inhibitors, and COX-11 (cyclooxygenase 11) inhibitors, can be used in conjunction with VS. Anti-angiogenesis agents include, for example, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published October 24,1996), WO 96/27583 (published March 7,1996), European Patent Application No. 97304971.1 (filed July 8,1997), European Patent Application No. 99308617.2 (filed Oct. 29, 1999), WO 98/07697 (published February 26,1998), WO 98/03516 (published January 29,1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published July 13,1994), European Patent Publication 931, 788 (published July 28,1999), WO 90/05719 (published May 31,1990), WO 99/52910 (published October 21,1999), WO 99/52889 (published Oct. 21, 1999), WO 99/29667 (published June 17,1999), PCT International Application No. PCT/IB98/01113 (filed July 21,1998), European Patent Application No. 99302232.1 (filed March 25,1999), Great Britain Patent Application No. 9912961.1 (filed Jun. 3, 1999), U.S. Provisional Application No. 60/148,464 (filed August 12,1999), U.S. Pat. No. 5,863,949 (issued January 26,1999), U.S. Pat. No. 5,861,510 (issued January 19,1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or AMP-9 relative to the other matrix-metalloproteinases (e.g., MAP-1, MMP-3, MMP-4, MMP-5, MMP-6, MMP-7, MMP-8, MMP-, MMP-ll, MMP-12, andMMP-13). Some specific examples of MMP inhibitors useful in the invention are AG-3340, RO 32-3555, and RS 13-0830.

Autophagy inhibitors include, but are not limited to chloroquine, 3-methyladenine, hydroxychloroquine (Plaquenil™), bafilomycin Al, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In some embodiments, medicaments which are administered in conjunction with a KRAS inhibitor and HDAC (e.g., HDAC3) inhibitor include any suitable drugs usefully delivered by inhalation for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines or pentamidine; antihistamines, e.g., methapyrilene; anti-inflammatories, e.g., beclomethasone, flunisolide, budesonide, tipredane, triamcinolone acetonide or fluticasone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g., amiloride; anticholinergics e.g., ipratropium, atropine or oxitropium; hormones, e.g., cortisone, hydrocortisone or prednisolone; xanthines e.g., aminophylline, choline theophyllinate, lysine theophyllinate or theophylline; and therapeutic proteins and peptides, e.g., insulin or glucagon Exemplary therapeutic agents useful for a combination therapy with a KRAS inhibitor and HDAC (e.g., HDAC3) inhibitor include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Other suitable therapeutic agents for coadministration with a KRAS inhibitor and HDAC (e.g., HDAC3) inhibitor also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, P-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated for co-administration with a KRAS inhibitor and HDAC (e.g., HDAC3) inhibitor include diuretics, vasopressin, agents affecting the renal conservation of water, rennin, angiotensin, agents useful in the treatment of myocardial ischemia, anti-hypertensive agents, angiotensin converting enzyme inhibitors, 0-adrenergic receptor antagonists, agents for the treatment of hypercholesterolemia, and agents for the treatment of dyslipidemia.

Other therapeutic agents contemplated for co-administration with a KRAS inhibitor and HDAC (e.g., HDAC3) inhibitor include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. Therapeutic agents used to treat protozoan infections, drugs used to treat Malaria, Amebiasis, Giardiasis, Trichomoniasis, Trypanosomiasis, and/or Leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, P-lactam antibiotics, an agent comprising an aminoglycoside, protein synthesis inhibitors, drugs used in the chemotherapy of tuberculosis, Mycobacterium avium complex disease, and leprosy, antifungal agents, antiviral agents including nonretroviral agents and antiretroviral agents.

Examples of therapeutic antibodies that can be combined with a KRAS inhibitor and HDAC (e.g., HDAC3) inhibitor include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab.

Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

### EXAMPLES

### L. Methods

### Cell Culture and Cell Lines

All cell lines were incubated at 37° C. and were maintained in an atmosphere containing 5% CO₂. Cells were tested for Mycoplasma (Lonza) using manufacturer's conditions and were deemed negative. Cells were grown in Dulbecco's modified Eagles medium (DMEM) plus 10% fetal bovine serum (Gibco) and were continuously maintained under antibiotic selection for stable cell lines. Proliferation assays were performed by plating 2×10\hat{ }3 cells per well of a 6-well plate, and cells were counted 5 days post-plating. Drug treatments were carried out using the following doses: entinostat 1 uM or 2 uM, RGFP966 10uM, TSA 0.5 uM, BRD3308 5 uM, trametinib 5 nM, 10 nM, and 25 nM, and MRTX1133 10 nM, 25 nM, and 50 nM, as indicated for the indicated treatment lengths. Media was changed and fresh drug added every 2 days.

Generating primary tumor cell lines Cell lines from KL primary tumors are not readily available due to the fact that, unlike KP tumor cells which lack p53, explanted KL primary tumor cells do not grow in culture, presumed to be from p53 activation-dependent growth arrest. To circumvent this issue, explanted KL tumor cells were immortalized before onset of growth arrest. Individual tumors were plucked from KL mice and, after dissociation and collagenase treatment, isolated cells were immortalized with SV40 T-antigen and subsequently purified by Epcam+ cell sorting to generate the epithelial lung tumor cell line KL LJE1. Specifically, to generate the KL LJE1 and KL LJE7 cell lines, individual primary tumors were dissected from the lungs of KL mice, mechanically dissociated, then digested for 45 min in digestion media (10% FBS, pen/strep, 1 mg/mLCollagenase/Dispase (Roche) in DMEM) at 37° C. Cells were strained through 70 uM nylon cell strainer, spun at 2000 rpm 5 min, resuspended in 1 mL complete media plus 5 uL Fungizone (Lifetech) and plated in a 24 well dish. 24h later, cells were infected by adding 1 mL T-antigen-expressing lentivirus to each well. 24h later, viral media was removed and replaced with complete media with Fungizone. Cells were cultured in Fungizone for 4 weeks, then Epcam+sorted.

CRISPR/Cas9 studies

Small Guide RNAs (sgRNAs) targeting mouse HDAC3 were selected using the optimized CRISPR design tool (crispr.mit.edu). The gSR gRNA sequence targeting NKX2-1 was obtained from Sanchez-Rivera et al. Nature 2014 (Ref. 74; incorporated by reference in its entirety), and the other gRNA targeting NKX2-1, g2, was designed with the GPP sgRNA designer (portals.broadinstitute.org/gpp/public/analysis-tools/sgrna-design). gRNAs targeting FGFR1 were designed with the Benchling program (www.benchling.com/crispr/). Guides with high targeting scores and low probability of off-target effects were chosen. At least three independent sgRNA sequences were tested for each gene. Oligonucleotides for sgRNAs were synthesized by IDT, annealed in vitro and subcloned into BsmbI-digested lentiCRISPRv.2-puro (Addgene 52961). Validation of guide specificity was assessed by Western blot. Assays were carried out within 6 passages of thawing early passage frozen cell stocks. Oligonucleotide sequences are listed in Table 1.

For FIG. 20C, the RNP system was used to deliver CRISPR/Cas9 using reagents from IDT: sgRNA targeting HDAC3 were ordered as crRNA. 50 uM of tracrRNA (IDT 1072533) was complexed with 50 uM of crRNA by heatshocking at 95° C. for 5 min and allowed to cool at room temperature for 5 min before being put on ice. 24 uM Cas9 nuclease (IDT 1081059) was added to the sgRNA and incubated at room temperature for 15 min and then placed on ice. Cells were trysinized, washed, and resuspended in 100ul of the Lonza nucleofector solution V, and the RNP complex and 3 uM electroporation enhancer (IDT 1075915) were added. The Lonza 2b Nucleofector system was used to electroporate the contents of this cell suspension using the T-030 program.

TABLE 1
Oligonucleotides

SEQ
ID
NO

CRISPR sgRNAs

| | | |
|---|---|---|
| Mouse: | | |
| Hdac3 g1 | CGTCAGTCCTGTCATACGT | 1 |
| Hdac3 g2-S | GGGGTCGTAGAAATACGCCA | 2 |
| Nkx2-1 | AAGAAAGTGGGCATGGAGGG | 3 |
| gSR-S | | |
| Nkx2-1 g2-S | CGCCGCCTACCACATGACGG | 4 |
| Fgfr1-S | CGACGATGACTCCTCCTCGG | 5 |
| p65 sg1 | AAAGTAAAGCCATTCGCCAG | 6 |
| p65 sg2 | GTCCATGGTCAGGGTCCCGG | 7 |
| Human: | | |
| HDAC3 sg1 | AGTCAATGTAGTCCTCGGAG | 8 |

qRT-PCR oligos

| | | |
|---|---|---|
| Mouse: | | |
| Fgfr1-F | AACTTGCCGTATGTCCAGATC | 9 |
| Fgfr1-R | AGAGTCCGATAGAGTTACCCG | 10 |
| Avpi1-F | GATAATCTGGGAGTGTGCAGG | 11 |
| Avpi1-R | GTCAGACGCTGTAGAACCAG | 12 |
| Ccl20-F | CCC AGC ACT GAG TAC ATC AAC | 13 |
| Ccl20-R | GTA TGT ACG AGA GGC AAC AGT C | 14 |
| Cxcl5-F | TGC CTA ATT TGG AGG TGA TCC | 15 |
| Cxcl5-R | TTG TCA GTG CCC AAT ATT TTC TG | 16 |
| Cxcl1-F | AGA ACA TCC AGA GCT TGA AGG | 17 |
| Cxcl1-R | CAA TTT TCT GAA CCA AGG GAG C | 18 |
| Cxcl2-F | CAG AAG TCA TAG CCA CTC TCA AG | 19 |
| Cxcl2-R | CTC CTT TCC AGG TCA GTT AGC | 20 |
| Cxcl3-F | TTT GAG ACC ATC CAG AGC TTG | 21 |
| Cxcl3-R | TTC TTG ACC ATC CTT GAG AGT G | 22 |
| Il-1a-F | CGC TTG AGT CGG CAA AGA AAT | 23 |
| Il-1a-R | GTG CAA GTC TCA TGA AGT GAG C | 24 |
| Cebpb-F | GGA TCA AAC GTG GCT GAG CG | 25 |
| Cebpb-R | GAT TAC TCA GGG CCC GGC TG | 26 |
| Cc15-F | CGT GCC CAC GTC AAG GAG TA | 27 |
| Cc15-R | TTC GAG TGA CAA ACA CGA CGA CTG C | 28 |
| Cxc110-F | CCA CGT GTT GAG ATC ATT GCC A | 29 |
| Cxc110-R | TGC GTG GTC TCA CTC CAG TT | 30 |
| G-Csf-F | TGC AGC CCA GAT CAC CCA GA | 31 |
| G-Csf-R | CAG CAG CTG CAG GGC CAT TAG | 32 |
| Gdf-15-F | AGT CCC AAC TCA ACG CCG AC | 33 |
| Gdf-15-R | GGA CCC CAA CTC CAC CTC TGG A | 34 |
| Nr1d1-F | TGC CAT GTT TGA CTT CAG CG | 35 |
| Nr1d1-R | GTT CTT CAG CAC CAG AGC C | 36 |
| Arntl-F | TCA AGA CGA CAT AGG ACA CTT | 37 |
| Arntl-R | GGA CAT TGG CTA AAA CAA CAG TG | 38 |
| Human: | | |
| CCL20-F | GGT GAA ATA TAT TGT GCG TCT CC | 39 |
| CCL20-R | ACT AAA CCC TCC ATG ATG TGC | 40 |
| CXCL5-F | TCT GCA AGT GTT CGC CAT AG | 41 |
| CXCL5-R | CAG TTT TCC TTG TTT CCA CCG | 42 |
| CXCL1-F | AAC CGA AGT CAT AGC CAC AC | 43 |
| CXCL1-R | CCT CCC TTC TGG TCA GTT G | 44 |
| CXCL2-F | CGC CCA AAC CGA AGT CAT AG | 45 |
| CXCL2-R | CTT CTG GTC AGT TGG ATT TGC | 46 |
| CXCL3-F | AAG TGT GAA TGT AAG GTC CCC | 47 |
| CXCL3-R | GTG CTC CCC TTG TTC AGT ATC | 48 |
| IL-1a-F | GGC GTT TGA GTC AGC AAA GAA GTC | 49 |
| IL-1a-R | TCA TGG AGT GGG CCA TAG CTT | 50 |
| CXCL10-F | GTC CAC GTG TTG AGA TCA TTG CT | 51 |
| CXCL10-R | GCC TCT GTG TGG TCC ATC CT | 52 |
| CCL5-F | ACA CCC TGC TGC TTT GCC TA | 53 |
| CCL5-R | CGG GTG ACA AAG ACG ACT GC | 54 |

### Lentiviral Production and Titering

Lentiviruses made from pLentiCRISPRv.2 were produced by co-transfection of the lentiviral backbone constructs and packaging plasmids pSPAX2 (Addgene 12260) and pMD2.G (Addgene 12259). Lipofectamine 2000 (Thermo Fisher Scientific) was used as a transfection reagent at a ratio of 3:1 lipofectamine/DNA. Viral supernatant was collected from 293 cells 48 post-transfection, 0.45 um-filtered, supplemented with polybrene, and applied to destination cells for 24h. Destination cells were allowed to recover from infection 24h before being subjected to selection with 2ug/ml. Resulting stably transduced lines were frozen down immediately after selection. Large-scale viral preps of Lenti Pgk-Cre (a gift from Tyler Jacks) were made by the University of Iowa Viral Vector Core. Titering: Lentiviral preps for mouse experiments (Pgk-Cre) were functionally titered by transduction of a reporter line (293-LSL-GFP), which turns on expression of GFP upon Cre-mediated recombination and allows quantitation of functional titers derived from the percent of GFP-positive cells.

### Mouse Studies

All procedures using animals were approved by the Salk Institute Institutional Animal Care and Use Committee (IACUC). All mice were maintained on the FVB/n background. Kras (Kras^{LSLG12D/+}; R26^{LSL;luc/luc}); KL (Kras^{LSLG12D/+}; Lkb1^{fl/fl};R26^{LSL;luc/luc}), KP (Kras^{LSLG12D/+}; p53^{fl/fl};R26^{LSLluc/luc}), and KPL (Kras^{LSLG12D/+}; Lkb1^{fl/fl};p53^{fl/fl};R26^{LSL;luc/luc}) mice in FVB/n have been previously described (Refs. 29, 36; incorporated by reference in their entireties). The Hdac3^{fl/fl} conditional floxed mouse has also been described (Ref. 23; incorporated by reference in its entirety). Hdac3^{fl/fl} was crossed into the FVB/n K background before crossing into the KL or KP genotypes to generate KL-HDAC3^{fl/fl} and KP-HDAC3^{fl/fl} experimental mice. All experiments used a mixture of female and male mice. Lentivirus expressing Cre recombinase (4×10\hat{ }5 pfu/mouse) was delivered by intratracheal intubation to each mouse to initiate lung tumorigenesis, according by the protocol of DuPage (Ref. 75; incorporated by reference in its entirety). Experimental endpoint was defined across experiments as the time point at which the experimental cohorts of KL or KP mice reached BLI tumor burden of 10\hat{ }8 mean photon flux, or earlier as indicated. At endpoint, all mice in that experiment were collected at that point. All animals at experimental endpoint were included for analysis of lung tumor burden and tumor size analysis. No animals were excluded from longitudinal BLI measurements and graphs.

### BLI Imaging

Bioluminescent imaging was performed biweekly using an IVIS Spectrum (Caliper Life Sciences) using Living Image software (Perkin Elmer). Mice were injected intraperitoneally with 150 mg/kg D-luciferin (Caliper Life Sciences), anesthetized with isoflurane and imaged both ventrally and dorsally 10 minutes post luciferin injection. The total lung photon flux for each animal is calculated by the combination of ventral and dorsal photon flux calculated within a region of interest (ROI) encompassing the thorax.

### Short-Term In Vivo Entinostat Treatment.

KL mice were intratracheally intubated with lentivirus expressing Cre recombinase to initiate tumorigenesis and imaged biweekly by BLI starting 4 weeks after Cre administration. When tumor burden in all mice was greater than 5×107 photons/see as determined by BLI, treatment was initiated. Mice were randomized and treated daily by oral gavage for 5 days with vehicle or 10 mg/kg entinostat. In vivo treatment doses were selected based on publications (39, 69-71). Entinostat was diluted to 1 mg/mL in vehicle (0.5% Methyl cellulose in water), vortexed, sonicated 10 minutes, and administered at 10 mg/kg. Drug and vehicle was administered starting at ˜9am daily throughout the duration of treatment. The experiment was terminated after 5 days of treatment, at which time primary lung tumors and liver were isolated by dissection and immediately flash frozen.

### In Vivo Entinostat and Trametinib Treatment

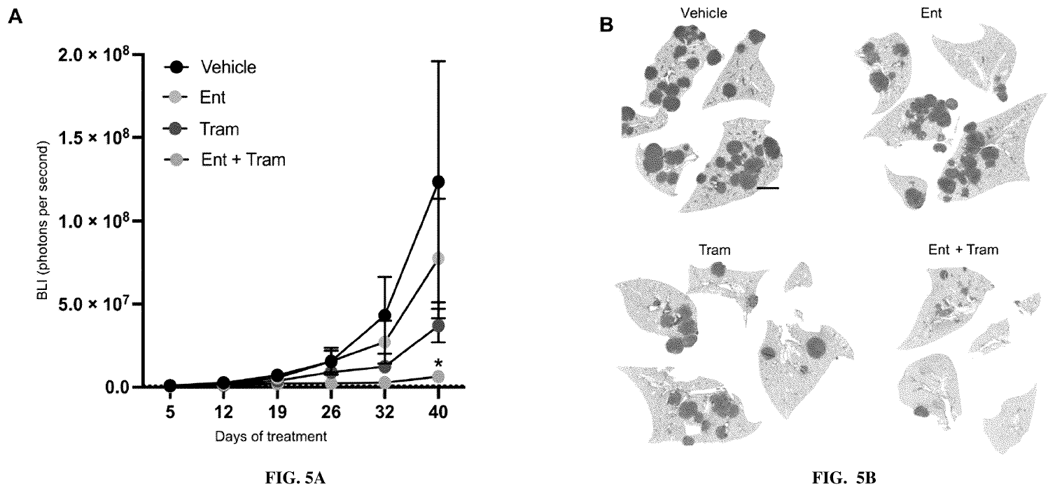

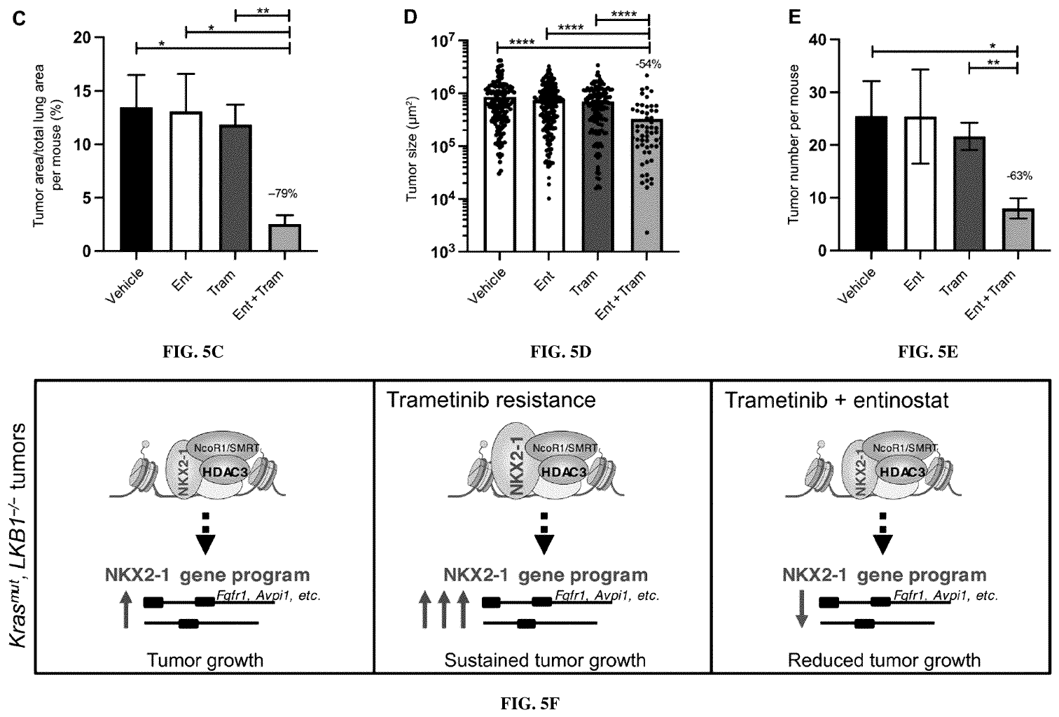

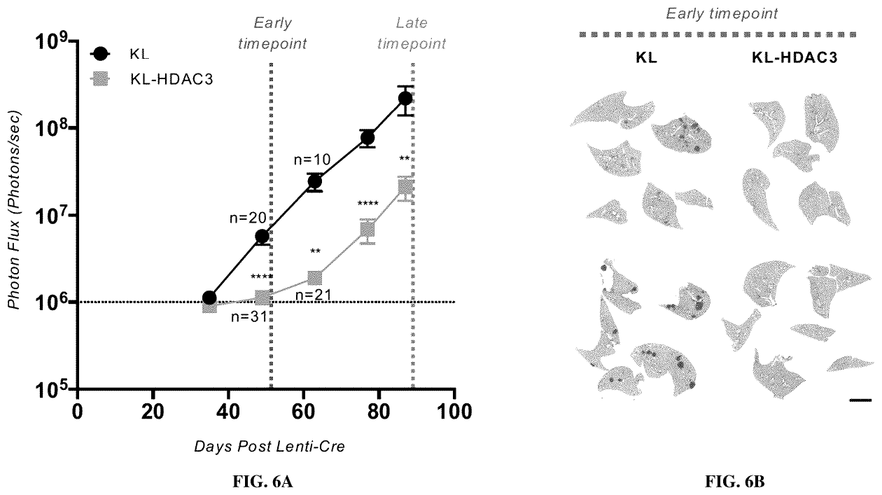

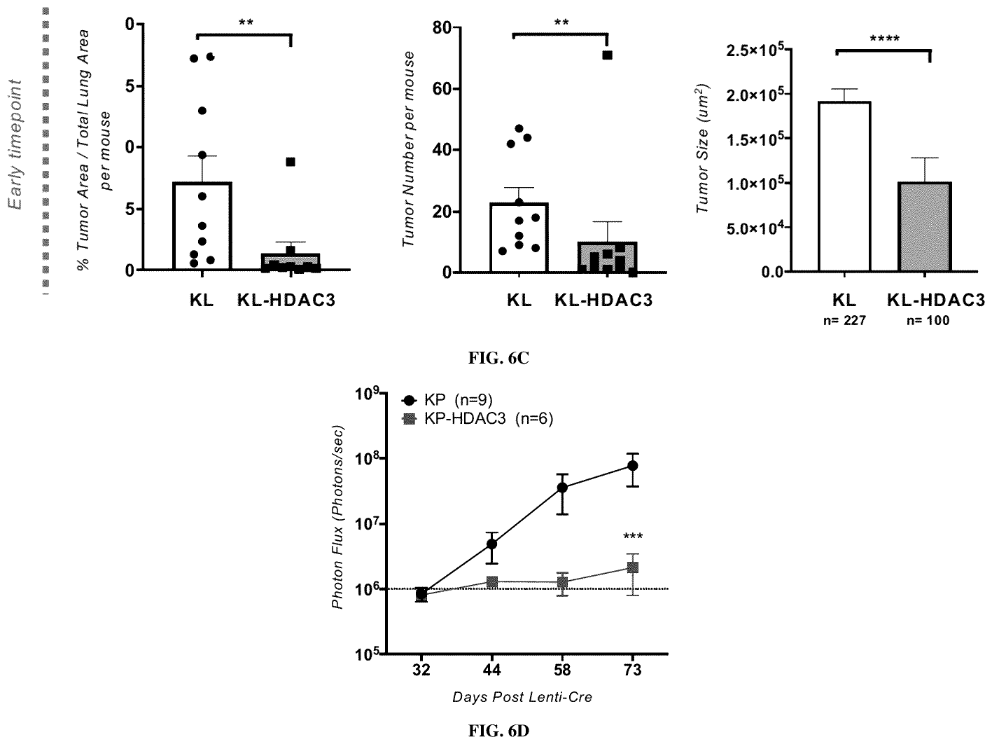

Mice were intratracheally intubated with Lentivirus expressing Cre recombinase to initiate tumorigenesis and imaged weekly starting 4 weeks post-Cre. Treatment was initiated at day 34 post-Cre (FIG. 5). In vivo treatment doses were selected based on publications (76-79). Entinostat was diluted to 1 mg/mL in vehicle (0.5% Methyl cellulose in water), vortexed, sonicated 10 minutes, and administered at 10 mg/kg. Trametinib was diluted to 2 mg/mL in Corn Oil, vortexed, sonicated 20 minutes, and this stock was stored up to 3 days at 4C. On the day of dosing, trametinib stock was diluted 1:10 in Corn Oil to 0.2 mg/mL and administered at 1 mg/kg. Drug vials were kept on a rack on a heat pad during dosing to maintain fluidity of the Corn Oil. Drug(s) and/or vehicle(s) were administered by sequential oral gavage starting at ˜9am daily throughout the duration of treatment, each mouse being gavaged twice on each dosing day (once to deliver entinostat or vehicle, and once to deliver trametinib or vehicle). On weekly imaging days, mice were given a drug holiday to mitigate any potential toxicity and reduce stress to the animals. Because mice were gavaged twice daily for an extended duration, an additional drug holiday was built into the dosing schedule starting after 3 weeks of treatment: mice were administered drug on a 4 days on, 1 day off, 1 day on, 1 day off (imaging day) schedule. Body weight of mice was recorded every two days, and did not indicate drug-induced toxicity in any treatment group throughout the treatment experiments. The experiment was terminated when the vehicle treatment group reached disease endpoint due to high lung tumor burden. Primary tumors were collected from the mouse with the highest tumor burden in each treatment group: mice were dosed with drug and sacrificed 2 hours later, at which point tumors were plucked from lungs and immediately flash frozen. To collect lungs at treatment endpoint, mice were sacrificed 2 hours after final dose of drug (˜9am), at which point lungs were inflated and formalin fixed.

For the 3-week duration study: When tumor burden was detectable in all mice by BLI, mice were randomized and treatment was initiated. Entinostat was administered at 5 mg/kg. Trametinib was administered at 1 mg/kg. Drug(s) were administered by oral gavage.

The 6-week duration study was carried out according to the same experimental design except for the following: To ensure an extended treatment duration, treatment was initiated after tumor burden was detected by BLI in >30% of mice in the study. Entinostat was administered at a dose of 10 mg/kg to assess systemic drug impact at a dose corresponding to potent tumor growth control. Because mice were gavaged twice daily for an extended duration, an additional drug holiday was built into the dosing schedule starting after 3 weeks of treatment; mice were administered drug using the following schedule: 4 days on, 1 day off, 1 day on, and 1 day off (imaging day).

### Immunohistochemistry and Image Analysis

Lungs from mice were collected at each experimental endpoint as noted in the Figures, and were fixed in formalin for 18-22 hrs, transferred to 70% ethanol and paraffin-embedded (FFPE) at the Tissue Technology Shared Resources at UCSD. 5 pm sections from FFPE tissues were prepared and stained with hematoxylin and eosin. For immunohistochemistry, slides were deparaffinized and rehydrated, and antigen retrieval was performed in citrate buffer for 13 min at high heat (−95° C.). Endogenous peroxidase activity was quenched with 10 min hydrogen peroxide in methanol. Using the ImmPress HRP Ig (Peroxidase) Polymer Detection Kits (Vector Labs), slides were blocked, incubated overnight with primary antibody diluted in blocking buffer, and secondary antibody steps were carried out according to the manufacturer's instructions. Staining was visualized with ImmPACT DAB peroxidase substrate (Vector Labs, SK-4105), and further counterstained with hematoxylin, dehydrated through ethanol and xylenes, and mounted with Cytoseal 60 (Thermo Scientific). H&E- and immunostained slides were scanned using a Perkin Elmer Slide Scanner (Panoramic MIDI Digital Side Scanner) for further downstream analysis using the Panoramic Viewer software, Inform v2.1 image analysis software (Cambridge Research and Instrumentation), or QuPath software (Ref. 80; incorporated by reference in its entirety).

### Lung Tumor Burden

Total lung tumor burden was quantitated from H&E sections using Inform v2.1 image analysis software (Cambridge Research and Instrumentation) in a non-biased manner. In brief, the Trainable Tissue Segmentation method was trained to identify tumor, normal lung, vessel and space. This program was then applied to all H&E images, and each of the resulting mapped images was then screened to verify that accurate tissue segmentation had occurred. The quantitation data from this analysis was then used to calculate the percentage of tumor area as normalized to total lung area (tumor area+normal lung area).

### Tumor Size Quantitation

Quantitation of each individual tumor was measured from H&E sections using morphometric analysis in Panoramic viewer software (Perkin Elmer), which calculates the size of each identified tumor by area in squared microns. The area of all tumors found in the 5 lobes of each mouse was exported and compiled to plot the number of tumors per mouse, and the average size of every tumor in the cohort.

mRNA Preparation and mRNA-Sequencing

mRNA was collected from cells harvested within 2 passages post-thaw. mRNA was isolated using the Quick-RNA Miniprep kit (Zymo Research), including DNase treatment. RNA integrity (RIN) numbers were determined using the Agilent TapeStation prior to library preparation. mRNA-seq libraries were prepared using the TruSeq RNA library preparation kit (version 2), according to the manufacturer's instructions (Illumina). Libraries were quantified, pooled, and sequenced by single-end 50 base pairs using the Illumina HiSeq 2500 platform at the Salk Next-Generation Sequencing Core. Raw sequencing data were demultiplexed and converted into FASTQ files using CASAVA (version 1.8.2).

mRNA Preparation and qRT-PCR

mRNA was prepared using the Quick-RNA Miniprep kit (Zymo Research), including DNase treatment. cDNA was synthesized from 2 μg of RNA using SuperScript III (Life Technologies), and qPCR was carried out with diluted cDNA, appropriate primers, and SYBR Green PCR master mix (ThermoFisher Scientific) using a C1000 Thermal Cycler (BioRad). Relative mRNA levels were calculated using the delta Ct method, using Tbp as an internal control. Table of primers used for qRT-PCR are listed in Table 1.

### Bioinformatic Analysis of RNA-Seq Data

Sequenced reads were quality-tested using the online FASTQC tool (bioinformatics.babraham.ac.uk/projects/fastqc) and aligned to the mouse mm10 genome using the STAR aligner version 2.4.0k (Ref. 81; incorporated by reference in its entirety). Raw gene expression was quantified across all annotated exons using HOMER (Ref. 82; incorporated by reference in its entirety), and differential gene expression was carried out using the getDiffExpression.pl command. Differentially expressed genes were defined as having a false discovery rate (FDR)<0.05 and a log 2 fold change >0.5.

GSEA was carried out with the GenePattern interface (genepattern.broadinstitute.org) using preranked lists generated from FDR values. Queried datasets used were gene lists from genes differentially expressed upon Tamoxifen-driven NKX2-1 KO in Kras tumors (Ref. 44; incorporated by reference in its entirety). Heatmaps were generated by clustering using the Cluster 3.0 program (log 2 transform data, center genes, Hierarchical clustering with average linkage) (Ref. 83; incorporated by reference in its entirety), and then visualized with Java TreeView version 1.1.6r4 (Ref. 84; incorporated by reference in its entirety).

ChIP-Sequencing Primary tumors. Individually dissected, flash frozen primary tumors were combined from 3 different mice into one pool of 130 mg of primary tumors per replicate per genotype. Equivalent masses of tumors were used from each of the three mice to ensure equal representation. Two independent pools of tumors per genotype were processed separately to generate two biological replicate pool of crosslinked, sonicated chromatin for ChIP. 4 independent ChIPs were performed on each pool of sonicated chromatin, and then pooled together to generate one replicate for ChIP-sequencing. To crosslink, tumors were dounce homogenized in crosslinking buffer (1% Formladehyde in PBS) and incubated with end-over-end rotation for 15 min at room temperature, and then quenched with 2.5M glycine 5 min. Samples were spun at 600g for 5 min, washed with cold PBS, and resuspended in ChIP buffer (RIPA) (see “Immunoprecipitation” for recipe) with protease inhibitors. Samples were sonicated in a Covaris LE 220 for 8 min (Duty Factor 2, 105 Watts, 200 cycles/burst), spun down, and the supernatant saved. For each ChIP, 100 uL lysate was combined with 900 uL ChIP buffer, while 50 uL was used for Input. 10ug of Hdac3 ab7030 antibody and 2ug H3K27ac ab4729 antibody was used for each ChIP. Lysate was incubated overnight with antibody. 20 uL washed and pre-blocked Protein A Dynabeads were incubated 2 hrs rotating with each sample at 4C. Washes were performed with 5 min incubations of each buffer while rotating at 4C. Samples were washed 3× with cold ChIP buffer, 1× with room temperature ChIP buffer, and 1× with room temperature TE pH 8, and then spun down. Elution of ChIP and Input samples was done by incubating samples with Elution buffer (50 mM Tris/Hcl pH 7.5, 10 mM EDTA, 1% SDS) overnight at 65C. Beads were pelleted and discarded, and 200 uL of eluate was combined with 194 uL low-EDTA TE and 100ug proteinase K, and incubated 2 hrs at 37C. 8 uL RNase A was added and samples incubated 30 min at 37C. Minelute PCR purification kit (Qiagen 28006) was used to isolate DNA, which was eluted in 15 uL EB at 55C. 4 ChIPs were combined into one sample for ChIP-sequencing.

KL LJE1 cells. ChIP-seq was carried out on DSG+Formaldehyde crosslinked, sonicated nuclear extracts. Cells were washed in PBS and then crosslinked by 30 min incubation in 2 mM DSG (Di(N-succinimidyl) glutarate, Thermo Fisher NC0054325). Aspirate, and incubate 15 min with 1% formaldehyde, before 5 min of quench with 125 mM glycine. Cells were washed in cold PBS, scraped, and spun down, and washed again in PBS before nuclei isolation. Nuclei were isolated by resuspension in CiA NP-Rinse 1 (50 mM HEPES, 140 mM NaCl, 1 mM EDTA, 10% glycerol, 0.5% NP-40, 0.25% Triton X100), incubated 10 min at 4C with end-over-end rotation, then centrifuged at 1,200g for 5 min at 4C. Samples were then resuspended in CiA NP-Rinse 2 (10 mM Tris pH 8.0, 1 mM EDTA, 0.5 mM EGTA, 200 mM NaCl), incubated 10 min at 4C with end-over-end rotation, and centrifuged at 1,200g for 5 min at 4C. Tubes were washed 2× with Covaris Shearing Buffer (0.1% SDS, 1 mM EDTA pH 8, 10 mM Tris HCl pH 8) to remove salt, centrifuged at 1,200g at 4C 3 min. Samples were diluted to a concentration of 2.5×10⁶ cells/130 uL in ChIP buffer (RIPA) (50 mM Tris-HCl pH 7.5, 140 mM NaCl, 1 mM EDTA, 1% Triton X-100, 0.1% NaDOC (sodium deoxycholate), 0.1% SDS) with protease inhibitors, and sonicated in a Covaris LE 220 for 8 min (Duty Factor 2, 105 Watts, 200 cycles/burst). Sonicated material was spun down and supernatant was used for ChIP. Lysate from 5 million cells was diluted in ChIP buffer to 1 mL final volume. 50 uL was used for Input. 10ug of Hdac3 CST-85057 antibody was used for each ChIP. Lysate was incubated overnight with antibody. 20 uL washed and pre-blocked Protein A Dynabeads were incubated 2 hrs rotating with each sample at 4C. Washes were performed with 5 min incubations of each buffer while rotating at 4C. Samples were washed 3× with cold ChIP buffer, 1× with room temperature ChIP buffer, and 1× with room temperature TE pH 8, and then spun down. Elution of ChIP and Input samples was done by incubating samples with Elution buffer (50 mM Tris/Hcl pH 7.5, 10 mM EDTA, 1% SDS) overnight at 65C. Beads were pelleted and discarded, and 200 uL of eluate was combined with 194 uL low-EDTA TE and 100ug proteinase K, and incubated 2 hrs at 37C. 8 uL RNase A was added and samples incubated 30 min at 37C. Minelute PCR purification kit (Qiagen 28006) was used to isolate DNA, which was eluted in 15 uL EB at 55C.

HDAC3 ChIP-seq was carried out on DSG+formaldehyde crosslinked, sonicated nuclear extracts from KL LJE1 cells. H3K27ac and H3K9ac ChIP-seq was carried out on formaldehyde crosslinked, sonicated nuclear extracts from KL LJE1 cells. Cells were washed in PBS and then incubated 15 min with 1% formaldehyde, before 5 min of quench with 125 mM glycine. Cells were washed in cold PBS, scraped, and spun down, and washed again in PBS before nuclei isolation. Nuclei were isolated by resuspension in CiA NP-Rinse 1 (50 mM HEPES, 140 mM NaCl, 1 mM EDTA, 10% glycerol, 0.5% NP-40, 0.25% Triton X100), incubated 10 min at 4C with end-over-end rotation, then centrifuged at 1,200g for 5 min at 4C. Samples were then resuspended in CiA NP-Rinse 2 (10 mM Tris pH 8.0, 1 mM EDTA, 0.5 mM EGTA, 200 mM NaCl), incubated 10 min at 4C with end-over-end rotation, and centrifuged at 1,200g for 5 min at 4C. Tubes were washed 2× with Covaris Shearing Buffer (0.1% SDS, 1 mM EDTA pH 8, 10 mM Tris HCl pH 8) to remove salt, centrifuged at 1,200g at 4C 3 min. Samples were diluted to a concentration of 2.5×106 cells/130 uL in ChIP buffer (RIPA) (50 mM Tris-HCl pH 7.5, 140 mM NaCl, 1 mM EDTA, 1% Triton X-100, 0.1% NaDOC (sodium deoxycholate), 0.1% SDS) with protease inhibitors, and sonicated in a Covaris LE 220 for 8 min (Duty Factor 2, 105 Watts, 200 cycles/burst). Sonicated material was spun down and supernatant was used for ChIP. Lysate from 5 million cells was diluted in ChIP buffer to 500 uL final volume. 50 uL was used for Input. 2ug of H3K27ac ab4729 antibody and 10 uL H3K9ac CST-9649 (lot 13) antibody was used for each ChIP. Lysate was incubated overnight with antibody. 20 uL washed and pre-blocked Protein A Dynabeads were incubated 2 hrs rotating with each sample at 4C. Washes were performed with 5 min incubations of each buffer while rotating at 4C. Samples were washed 4× with cold ChIP buffer, and 1× with room temperature TE pH 8, and then spun down. Elution of ChIP and Input samples was done by incubating samples with Elution buffer (50 mM Tris/Hcl pH 7.5, 10 mM EDTA, 1% SDS) overnight at 65C. Beads were pelleted and discarded, and 200 μL of eluate was combined with 194 uL low-EDTA TE and 100ug proteinase K, and incubated 2 hrs at 37C. 8 uL RNase A was added and samples incubated 30 min at 37C. Minelute PCR purification kit (Qiagen 28006) was used to isolate DNA, which was eluted in 15 uL EB at 55C.

### Bioinformatic Analysis of ChIP-Seq Data

Sequenced reads were aligned to the mouse mm10 genome using the STAR aligner version 2.4.0k. HOMER was used for data processing. For KL LJE1 cell line ChIP-seq data, peaks were called using the getDifferentialPeaksReplicates.pl command using HDAC3 ChIP-seq data from NT cells as target (−t), HDAC3 ChIP-seq data from HDAC3 KO cells as background (−b), and Input sequencing data from NT cells as input (−i), with—style factor and —F 3. For primary tumor ChIP-seq data, peaks were called for each replicate individually using the findPeaks command with parameters-style factor −F 3, using HDAC3 ChIP-seq as target and Input sequencing data as Input (−i). Peaks were merged using the mergePeaks command to generate a consolidated file containing all HDAC3 ChIP-seq peaks identified in KL and KP tumors. The getDifferentialPeaks command with −F 3-same was used to identify peaks bound in both KL and KP tumors. The annotatePeaks.pl command with the —ghist —hist 25 option was used to visualize binding at each peak independently across samples, and Java TreeView was used to visualize the output. The annotatePeaks.pl command with —hist 25 was used to plot average reads across all peaks relative to peak center for each replicate separately. BedGraph files were also generated and visualized with Integrative Genomics Viewer (IGV) version 2.5.1.

### Western Blots

Protein lysates in CST buffer (20 mM Tris pH 7.5, 150 mM NaCl, 1% Triton X-100, 50 mM NaF, 1 mM EDTA, 1 mM EGTA, 2.5 mM PyroPhosphate, 2 mM beta-glycerol-phosphate, 1 mM orthovanadate, 0.01 mM Calyculan A) with protease inhibitors were equilibrated for protein levels using a BCA protein assay kit (Pierce), resolved on 8% SDS-PAGE gels, and transferred to membrane. Membranes were blocked in milk, incubated o/n at 4C in diluted primary antibody, washed with TBS-T, incubated 1 hr in secondary antibody diluted in in TBS-T plus milk, washed in TBS-T, and developed using SuperSignal ECL. Secondary antibodies: anti-rabbit (Millipore AP132P) and anti-mouse (Millipore AP124P). Nuclear fractions were isolated using a NE-PER nuclear and cytoplasmic extraction kit (Thermofisher) under manufacturers conditions.

### Immunoprecipitation

Immunoprecipitation was carried out on DSP-crosslinked, sonicated nuclear lysates. Cells were washed in PBS and then crosslinked by 30 min incubation in 1 mM DSP (dithiobis(succinimidyl propionate), Thermo Scientific 22585), followed by 5 min of quench with 2.5M glycine. Cells were washed in PBS, scraped, and spun down, and washed again in PBS before nuclei isolation. Nuclei were isolated by resuspension in CiA NP-Rinse 1 (50 mM HEPES, 140 mM NaCl, 1 mM EDTA, 10% glycerol, 0.5% NP-40, 0.25% Triton X100), incubated 10 min at 4C with end-over-end rotation, then centrifuged at 1,200g for 5 min at 4C. Samples were then resuspended in CiA NP-Rinse 2 (10 mM Tris pH 8.0, 1 mM EDTA, 0.5 mM EGTA, 200 mM NaCl), incubated 10 min at 4C with end-over-end rotation, and centrifuged at 1,200g for 5 min at 4C. Tubes were washed twice with Covaris Shearing Buffer (0.1% SDS, 1 mM EDTA pH 8, 10 mM Tris HCl pH 8) to remove salt, centrifuged at 1,200g at 4C 3 min. Samples were diluted to a concentration of 2.5×10⁶ cells/130 uL in ChIP buffer (RIPA) (50 mM Tris-HCl pH 7.5, 140 mM NaCl, 1 mM EDTA, 1% Triton X-100, 0.1% NaDOC (sodium deoxycholate), 0.1% SDS) with protease inhibitors, and sonicated in a Covaris LE 220 for 8 min (Duty Factor 2, 105 Watts, 200 cycles/burst). Sonicated material was spun down and supernatant was used for IP: 400 uL material was incubated with 3 uL NKX2-1 antibody (Abcam ab76013) per IP overnight with rotation at 4C. 20 uL prewashed Protein A Dynabeads were added per tube, and incubated 4 hrs at 4C with rotation. Samples were washed 5× with CST buffer (see western blot section) before adding 25 uL 6× loading dye and 50 uL CST per tube, and eluting by boiling for 5 min. Input and IP samples were subsequently assessed by western blot.

### Cytokine Array ELISA

Cytokine Array Elisa was carried out according to manufacturer instructions from the Proteome Profiler Mouse XL Cytokine Array (ARY028) from R&D Systems. Cell culture supernatant from KL LJE1 NT and HDAC3 KO cells was profiled. Media was collected after 3 days of cell culture without media change. Media was spun down to remove cell debris, and 500 uL was profiled per replicate. Signal intensity per spotted cytokine was quantitated using ImageJ (imagej.nih.gov/ij/download.html), and data was normalized to the positive controls on each array.

### Transient Transfection

p65-expressing DNA constructs RelA-Flag (22) (Addgene 20012) and GFP-RelA (21) (Addgene 23255) were transiently transfected into KL LJE1 cells using Lipofectamine 2000 (Thermo Fisher Scientific) according to the manufacturer's protocol. Cells were treated with drug as indicated 24 hours after transfection.

### Flow Cytometry Immune Profiling.

Tumor digestion and cell isolation: Lungs were removed from the chest, washed with PBS, dried on paper towels and then tumor nodules were excised from adjacent tissue and both adjacent tissue and nodules were weighed. Nodules or adjacent tissue was minced into small pieces and suspended in digestion media consisting of RPMI 1640 with 2% FBS, 0.5 ug/mL of DNase I and 1 unit/mL collagenase Type I (Sigma-Aldrich Cat #0000137295) and placed in an incubator at 37° C. for 45 minutes, shaking once during digestion. Lungs were then mashed against 70 μM cell strainers (VWR Cat #10199-657) to filter and then red blood cells were lysed using ACK lysis buffer, mixed with RPMI 1640 containing 10% FBS and 1% pen-strep, centrifuged at 400g for 5 minutes at 4° C. to obtain a single-cell suspension.

Flow Cytometry, cell sorting, and antibodies: Prior to staining, single cell suspensions were incubated on ice for 10 minutes with Fc receptor-blocking anti-CD16/32 (BioLegend Cat #101301). Cell suspensions were first stained for 5 minutes at room temperature with LIVE/DEAD® Fixable Red Dead Cell Stain Kit (ThermoFisher Cat #L23102). Surface proteins were then stained for 30 minutes at 4° C. in FACS buffer (PBS containing 2% FBS and 0.1% NaN3 (MP Bio Cat #2102891-CF)). For ex-vivo cytokine production, cell suspensions were re-suspended in RPMI 1640 containing 10% FBS with 50 ng/ml PMA (Phorbol 12-myristate 13-acetate) (Sigma Cat #P8139) and 3 μM Ionomycin (Sigma Cat #10634) in the presence 2.5 pg/ml Brefeldin A (BioLegend Cat #420601) for 4 hours at 37° C. For surface marker staining on stimulated cell suspensions, cells were processed as described above. Cells were fixed in BD Cytofix/Cytoperm (BD Cat #554714) for 30 minutes at 4° C., then washed with 1×Permeabilization buffer (Invitrogen Cat #00-8333-56) to measure intracellular cytokine staining and cells were fixed with Foxp3/Transcription Factor Fixation/Permeabilization buffer (Invitrogen Cat #00-5521-00) for 30 minutes at 4° C., then washed with 1×Permeabilization buffer (three times) to assess nuclear protein staining. Subsequently, cells were stained for 30 minutes at 4° C. with intracellular antibodies. The LSR-II flow cytometer (BD Biosciences) was employed to process samples, and FlowJo V10 (TreeStar RRID:SCR_008520) was used to analyze data.

The following list of antibodies and their concentration against mouse proteins were employed: αCD45 (1:400; BioLegend Cat #103147, RRID:AB 2564383), αCD3ε (1:300; Thermo Fisher Scientific Cat #46-0032-82, RRID:AB_1834427), αCD4 (1:300; BioLegend Cat #100406, RRID:AB_312691), αCD8a (1:300; BD Biosciences Cat #741811, RRID:AB_2871149), αCD11b (1:800; BD Biosciences Cat #612801, RRID:AB_2870128), αLy6G (1:300; BioLegend Cat #127624, RRID:AB_10640819), αTNFα (1:300; (BioLegend Cat #506305, RRID:AB_315426), αKi67 (1:300; Thermo Fisher Scientific Cat #56-5698-82, RRID:AB_2637480), aIFNy (1:300; BioLegend Cat #505829, RRID:AB_10897937), aFoxP3 (1:200, BioLegend Cat #126404, RRID:AB_1089117), αPD-1 (1:300, BioLegend Cat #135219, RRID:AB_11125371), αCD44 (1:300, BioLegend Cat #103012, RRID:AB_312963), αCD62L (1:300, BD Biosciences Cat #740218, RRID:AB_2739966), aSlamF6 (1:300, BD Biosciences Cat #745250, RRID:AB_2742834), αTim3 (1:300, BioLegend Cat #119727, RRID:AB_2716208), αCD101 (1:300, Thermo Fisher Scientific Cat #25-1011-82, RRID:AB_2573378).

TCGA analysis of Firehose LUAD dataset The results shown are in whole based upon data generated by the TCGA Research Network: cancergenome.nih.gov/. TCGA datasets were queried using cBioPortal (cbioportal.org) (Refs. 85-86; incorporated by reference in their entireties). Methods for data generation, normalization and bioinformatics analyses were previously described in the TCGA LUAD publication (Cancer Genome Atlas Research 2014). mRNA data used for this analysis was RNA Seq V2 RSEM with z-score thresholds of 1.8.

### Homer Motif Enrichment Analysis

Homer motif enrichment analysis: homer.ucsd.edu/homer/motif/.

### Web-Based Analysis Tools.

Pathway analysis was performed with Enrichr: amp.pharm.mssm.edu/Enrichr. 4-way Venn diagrams were plotted using Venny 2.1 (Oliveros, J.C. (2007-2015) Venny. An interactive tool for comparing lists with Venn's diagrams, (bioinfogp.cnb.csic.es/tools/venny/). Area-proportional Venn diagrams were plotted using BioVenn (biovenn.nl).

### Antibodies and Reagents

Western blotting. Antibodies from Cell Signaling Technologies (Denvers, MA USA) were diluted 1:1,000: Hdac3 CST-85057, Fgfrl CST-9740, ERK CST-4695, p300 CST-70088, SIK2 CST-6919, acetyl-NF-kB p65 (Lys310) CST-12629, NF-kB p65 CST-8242, or 1:2,000: phospho-ERK Thr202/Tyr2O4CST-4370. Nkx2-1 was from Abcam ab76013 and was diluted 1:1,500. From Sigma-Aldrich, anti-actin (#A5441) was diluted 1:10,000. ChIP. Hdac3 from Abcam ab7030 was used on primary tumors, and Hdac3 from Cell Signaling Technologies CST-85057 was used on KL LJE1 cells. Hdac3 from Cell Signaling Technologies CST-85057, H3K27ac from Abcam ab4729, H3K9ac from Cell Signaling Technologies CST-9649.

IP. Nkx2-1 raised in rabbit from (Abcam ab76013) was used to immunoprecipitate, and HDAC3 raised in mouse (CST-3949) was used to detect co-immunoprecipitated HDAC3. Abcam antibody ab5690 (1:150) was used to detect CD3, and ab198505 (1:100) was used to detect Cxc15/6. Cell Signaling Technologies CST-70076 was used (1:250) to detect F4/80. R&D Systems AF2225 was used (5ug/mL) to detect NKp46/NCR1. BioXcell BE0075-1 was used (1:1,000) to detect Ly6g.

Drugs. Entinostat (S1053), RGFP966 (57229), TSA (S1045), and BRD3308 (58962) was obtained from Selleck Chemicals. Trametinib was obtained from LC Laboratories (T-8123). MRTX1133 (CT-MRTX1133) was obtained from Chemietek.

### Statistical Analyses

Statistical analyses are described in each FIG. and were all performed using Graph Pad Prism 9. Results are expressed as mean±s.e.m. unless otherwise indicated.

### II. Results

### HDAC3 is Essential for Lung Tumorigenesis In Vivo

To assess the role of HDAC3 in solid tumors in vivo, two mouse models were utilized engineered to recapitulate the most common subtypes of Kras-mutant Non-Small Cell Lung Cancer (NSCLC); mutant Kras combined with LKB1 loss, Kras^{LSL-G12D/+}STK11^{-/-} (KL), and mutant Kras combined with p53 loss, Kras^{LSL-G12D/+}p53^{-/-} (KP). Mice harboring Kras^{LSL-G12D/+}/+STK11^{L/L}, ROSA26^{LSL-luciferase}, with or without conditional HDAC3Y (KL-HDAC3) were first examined. In these mice, intratracheal administration of lentivirus expressing Cre recombinase (Lenti-Cre) simultaneously activates Kras^{G12D} and deletes LKB1 (STK11) to initiate tumorigenesis in the lung epithelium, and for those bearing HDAC3^{L/L}, coincidentally deletes HDAC3. Simultaneously, Cre recombinase induces expression of firefly luciferase in infected cells, allowing for noninvasive longitudinal bioluminescence imaging (BLI) of NSCLC tumor development in the whole animal as we have reported previously (Refs. 29, 31, 35-36; incorporated by reference in their entireties). Tumor growth was markedly reduced in KL-HDAC3 mice compared to KL littermate controls at both early and late timepoints, exhibiting significantly less tumor area, tumor number and smaller tumor size (FIG. 1A-C, 6A-C). Thus, it can be concluded that HDAC3 supports tumor initiation and tumor growth in the KL model of NSCLC. Employing a similar experimental design, mice harboring Kras^{LSL-G12D/+}, p53^{L/L}, ROSA26^{LSL-luciferase}, HDAC3^{L/L} (KP-HDAC3) were generated to test the role of HDAC3 in the KP model of NSCLC. Tumor growth was dramatically reduced in KP-HDAC3 mice compared to KP littermate controls, with significantly less tumor area and smaller tumor size, and a trend toward smaller tumor number (FIG. 1D-F, 6D). It was concluded that HDAC3 is of critical importance for growth of NSCLC tumors driven by both KL and KP genotypes.

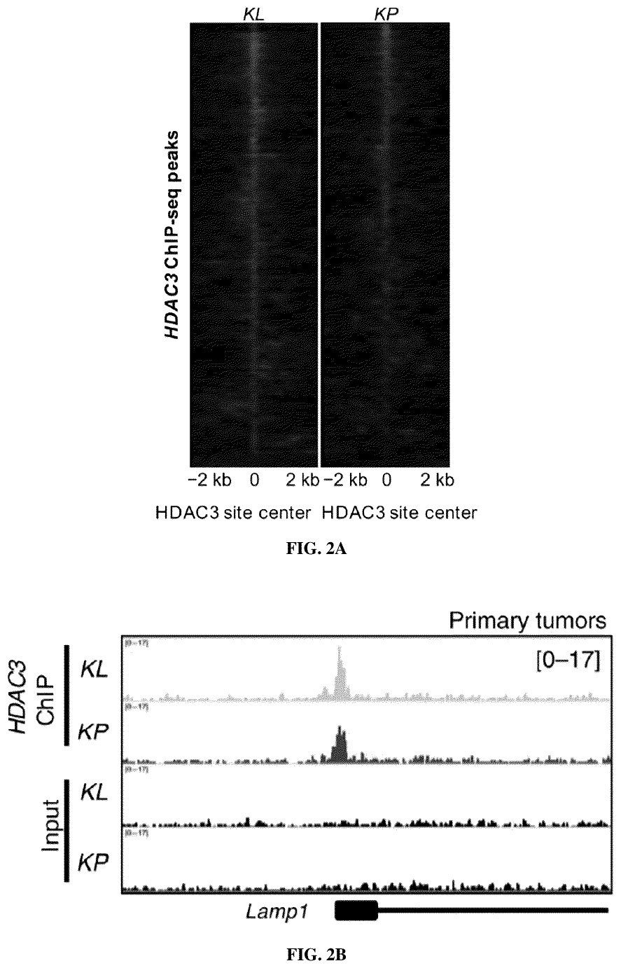

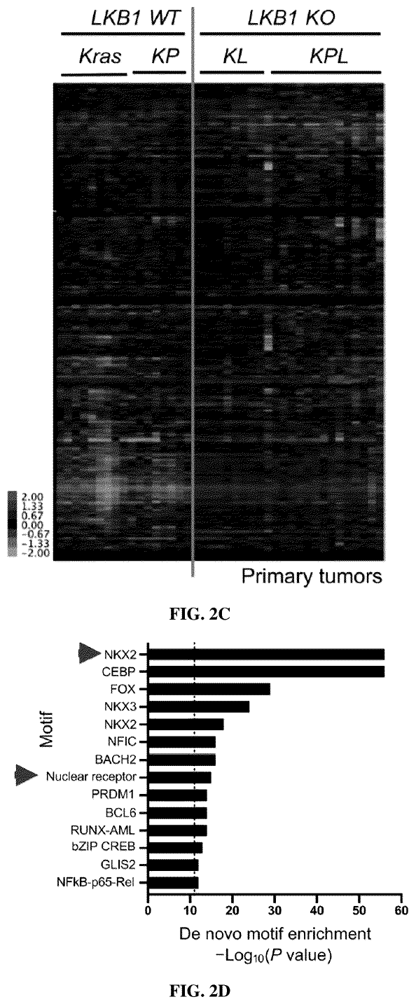

HDAC3 genome occupancy in primary tumors predicts cooperation with NKX2-1 Transcriptional programs regulated by HDAC3 are dramatically tissue context specific, in a manner often dictated by the identity of the cooperating transcription factors (Ref. 19; incorporated by reference in its entirety). Therefore, to understand the molecular mechanism mediating the function of HDAC3 in NSCLC, experiments were conducted during development of embodiments herein to to identify the transcription factors with which HDAC3 cooperates in this cellular context HDAC3 ChIP-seq was performed on KL and KP primary tumors to identify genome-wide, endogenously bound HDAC3 target loci in vivo (FIG. 2A-B, 7A). 1522 peaks were bound by HDAC3 in both KL and KP tumors (FIG. 2A), corresponding to 753 non-redundant genes with at least one HDAC3 binding site within +/−25 kilobases of the Transcription Start Site (TSS).

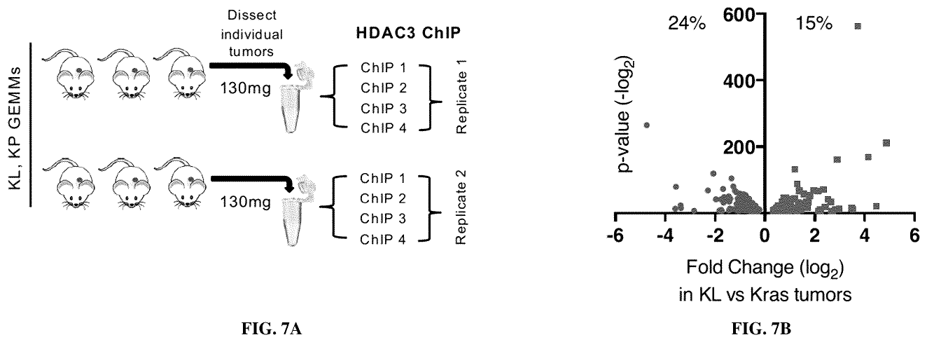

The expression of these 753 direct HDAC3 target genes was plotted across RNA sequencing (RNA-seq) data from individual primary lung tumors dissected from four different Kras-mutant genetically engineered mouse models of NSCLC; the Kras, KP, KL, and KPL (FIG. 2C) models. This analysis revealed an unexpected LKB1-dependent gene expression pattern, where gene expression was distinctly different in LKB1 wildtype (Kras, KP) versus LKB1 knockout (KL, KPL) tumor cells. 39% of HDAC3 direct target genes were differentially expressed between Kras and KL tumors (FIG. 7B). LKB1-mutant tumors exhibit unique gene expression signatures clinically (Refs. 37-38; incorporated by reference in their entireties), but involvement of HDAC3 in LKB1-specific gene expression is unknown. The data thus far suggest that LKB1 loss affects the expression of HDAC3 target genes, suggesting an important role for HDAC3 in these tumors.

Experiments were conducted during development of embodiments herein to identify the transcription factors with which HDAC3 cooperates in the lung tumor context. HDAC3 enzymatic activity requires interaction with Nuclear Receptor (NR) corepressors (Ref. 39; incorporated by reference in its entirety) and, consistently, de novo motif enrichment analysis of the HDAC3 ChIP-seq binding sites (FIG. 2A) revealed the classical motif recognized by a number of NRs (FIG. 2D). However, the most enriched de novo motif was that of the transcription factor NK2 homeobox 1/Thyroid Transcription Factor 1 (NKX2-1/TTF-1) (FIG. 2D). This indicated an unexpected functional overlap between HDAC3 and NKX2-1. This finding was particularly relevant to the NSCLC context, as NKX2-1 is highly expressed in up to 85% and amplified in up to 15% of human lung adenocarcinoma (LUAD) cases, to the extent that it is used as a clinical biomarker of the disease (Refs. 26, 40-43; incorporated by reference in their entireties).

Functionally, NKX2-1 is considered a lineage-survival oncogene, and it has an established role enforcing a lineage-specific differentiation program in lung and LUAD (Refs. 41, 44-46; incorporated by reference in their entireties). Lineage-specific oncogenic transcription factors are appreciated addictions of cancer but often lack entry points for therapeutic intervention (Ref. 47; incorporated by reference in its entirety), as is the case for NKX2-1 in the lung cancer context. NKX2-1 is an appreciated but undruggable transcriptional addiction of lung adenocarcinoma, and identifying druggable regulators of NKX2-1 function is of great interest. Moreover, HDAC3 has recently been implicated as a regulator of lineage specification in normal T-cells and glial cells (refs. 48-49; incorporated by reference in their entireties).

HDAC3 Cooperates with NKX2-1 to Regulate the Expression of a Common Set of Target Genes

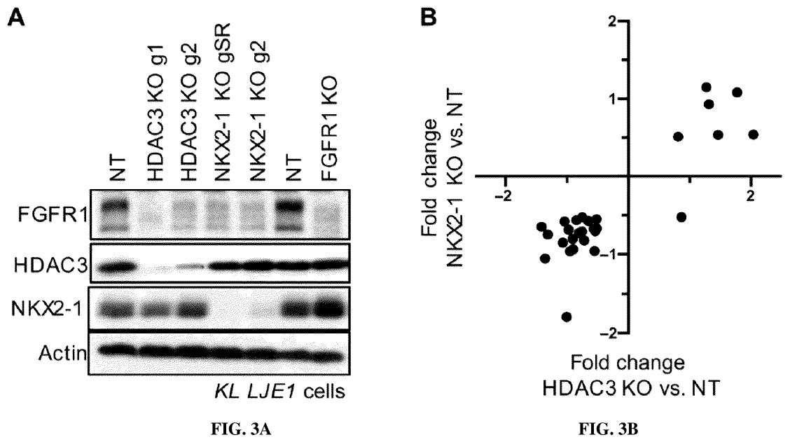

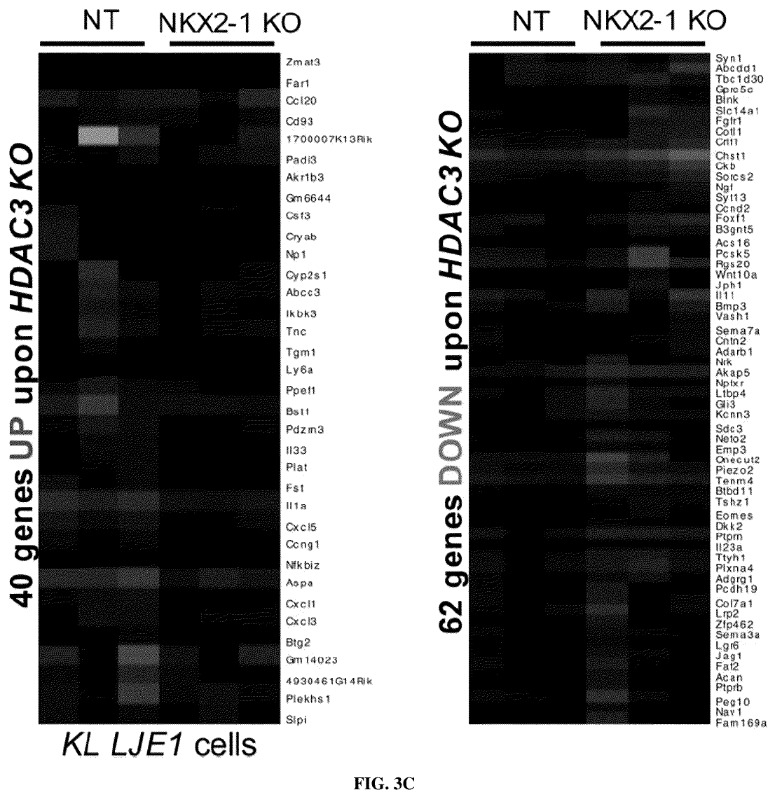

Experiments were conducted during development of embodiments herein to assess if HDAC3 and NKX2-1 coordinately control a common set of target genes in NSCLC cells. An immortalized cell line, KL LJE1 cells, was created from an explanted KL primary lung tumor, as described in Methods. Using CRISPR/Cas9, HDAC3 or NKX2-1 were then disrupted in KL LJE1 cells. KL LJE1 cells were infected with lentivirus expressing Cas9 and sgRNA directed against a Non-Targeting sequence (NT), HDAC3, or NKX2-1. Two independent sgRNAs directed against HDAC3 or NKX2-1 were used. Subsequent puromycin selection generated a pooled population of NT, HDAC3 knockout (KO), or NKX2-1 KO cells, and immunoblotting verified deletion (FIG. 3A).

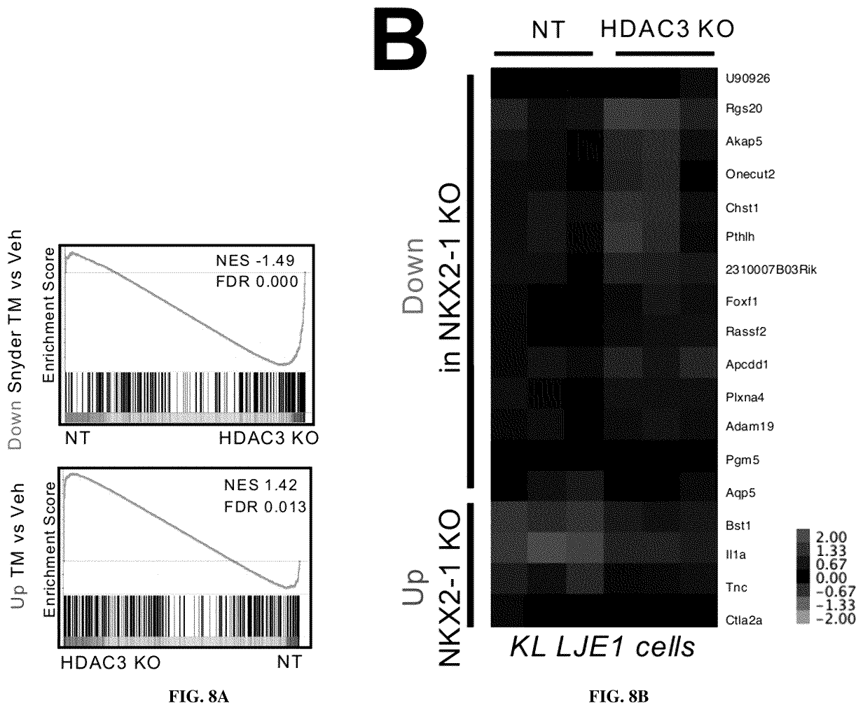

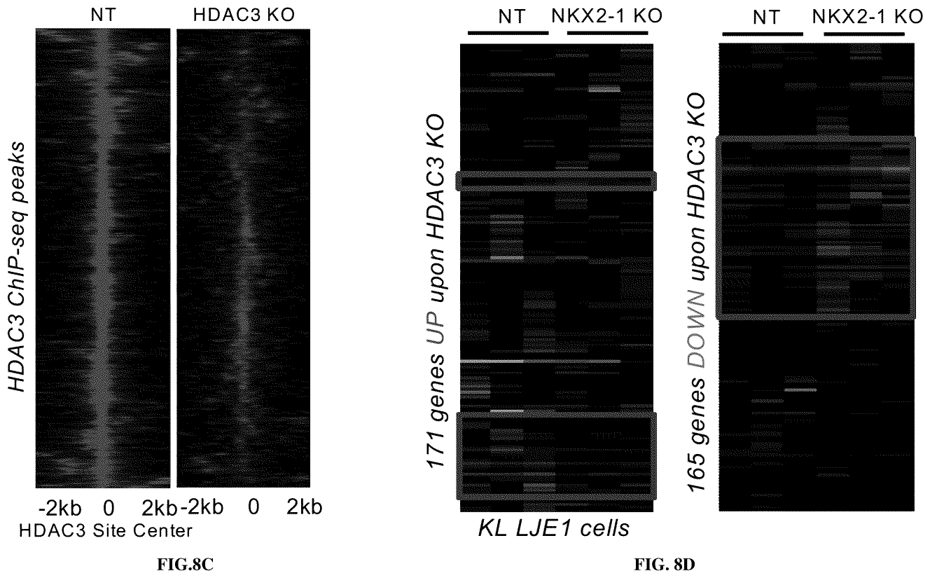

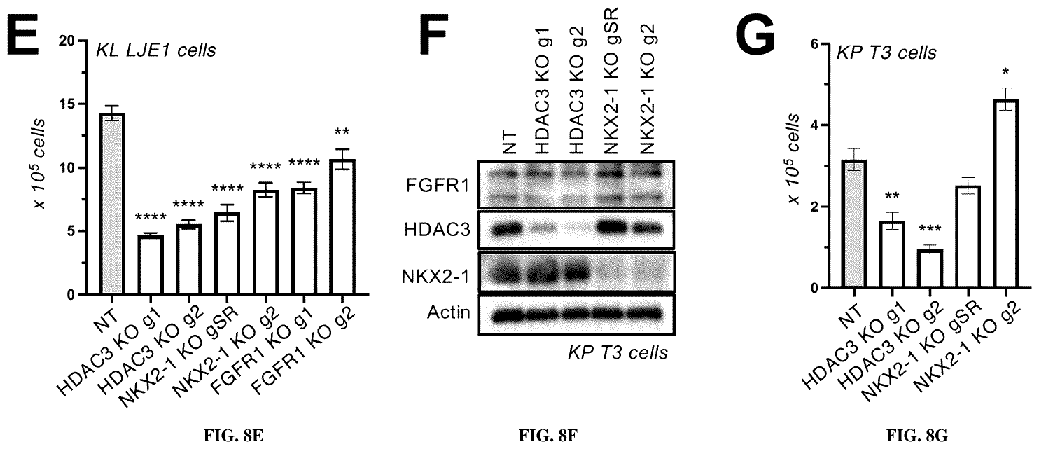

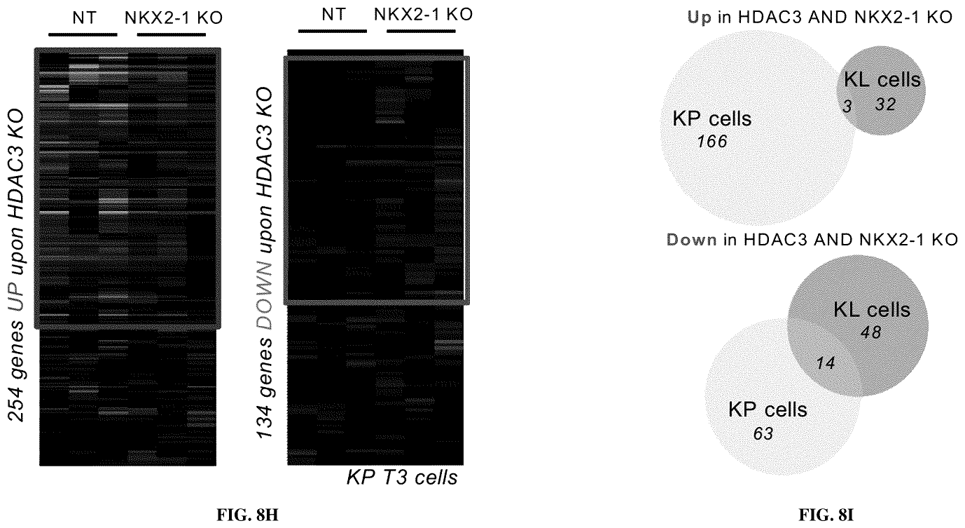

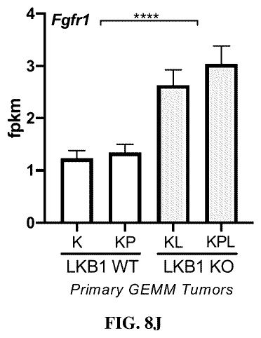

These cell lines were profiled by RNA-seq. Comparison between RNA-seq datasets identified a common set of genes deregulation upon HDAC3 KO and NKX2-1 KO, and that each of these knockouts impact gene expression changes with the same directionality in KL NSCLC cells (FIG. 3B). Comparison with published data from Kras^{mut} tumors deleted for NKX2-1 (Ref. 44; incorporated by reference in its entirety) verified that, for target genes co-regulated by both factors, HDAC3 predominantly promotes the gene expression program driven by NKX2-1 (FIG. 8A) and loss of either protein results in reduced expression of common target genes.

Experiments were conducted during development of embodiments herein to query which fraction of the NKX2-1 transcriptional program is regulated by HDAC3. Of the genes most de-regulated upon NKX2-1 KO, 83% were also modulated upon HDAC3 KO (FIG. 8B), including the established NKX2-1 target Tnc (Ref. 50; incorporated by reference in its entirety), indicating that the most NKX2-1-dependent genes are nearly all under the control of HDAC3. To query the extent to which HDAC3 is involved in the regulation of NKX2-1 target genes across a broader set of genes, this analysis was extended to the 68 genes deregulated upon NKX2-1 KO by the stronger sgRNA (gSR), and it was found that 72% of NKX2-1 target genes were also modulated upon HDAC3 KO in these cells. This indiucates that nearly three quarters of the NKX2-1 transcriptional program is co-regulated by HDAC3. To identify the direct targets of endogenous HDAC3 in these cells, HDAC3 ChIP-seq (FIG. 8C) was performed on NT and HDAC3 KO KL LJE1 cells. Overlay with the RNA-seq data revealed that 31% of the genes regulated by both NKX2-1 and HDAC3 were associated with at least one HDAC3 ChIP-seq peak, indicative of direct regulation by HDAC3. Notably, HDAC3 was not observed to be binding at or near the NKX2-1 genomic locus in HDAC3 ChIP-seq experiments.

Experiments were conducted during development of embodiments herein to determine what fraction of the total HDAC3 transcriptional response in these cells is regulated by NKX2-1. For genes differentially expressed upon HDAC3 KO, gene expression from NT and NKX2-1 KO cells was plotted (FIG. 3C, 8D). Of the 171 genes upregulated upon HDAC3 KO, 21% were also upregulated upon NKX2-1 KO, but only 3% were downregulated upon NKX2-1 KO. Of the 165 genes downregulated upon HDAC3 KO, 38% were also downregulated upon NKX2-1 KO, and none were upregulated upon NKX2-1 KO. NKX2-1 regulates the expression of ˜30% of the HDAC3-dependent genes in KL NSCLC cells. Notably, genes both activated and repressed by NKX2-1/HDAC3 are direct targets of HDAC3, indicating that HDAC3 is not solely acting as a canonical repressor on the NKX2-1-regulated genes in KL cells.

HDAC3 and NKX2-1 predominantly promote the expression of a set of common target genes in KL cells. One co-regulated target of both NKX2-1 and HDAC3 (FIG. 3C) is Fgfr1, which was selected for further validation. Fibroblast Growth Factor Receptor 1 (FGFR1), is one of four receptor tyrosine kinases that make up the FGFR protein family. FGFRs, receptors for Fibroblast Growth Factors (FGFs), have been widely implicated in promoting tumor growth, and multiple small molecule inhibitors of FGFRs are in various stages of development as cancer therapies (Refs. 51-52; incorporated by reference in their entireties). RNA-seq analysis identified that Fgfr1 mRNA was downregulated upon both NKX2-1 KO or HDAC3 KO. Western blotting revealed that both HDAC3 and NKX2-1 are required for maintenance of FGFR1 protein expression in KL cells (FIG. 3A). HDAC3, NKX2-1, and FGFR1 all support tumor cell growth in KL LJE1 cells (FIG. 8E).

Considering the LKB1-dependent expression pattern of HDAC3 target genes in primary lung tumors (FIG. 2C), experiments were conducted during development of embodiments herein to test the impact of HDAC3 and NKX2-1 in an LKB1-wildtype cell line derived from a KP GEMM tumor, KP T3 cells (Ref. 31; incorporated by reference in its entirety). Unlike in KL cells, neither HDAC3 KO nor NKX2-1 KO impacted FGFR1 protein expression in KP T3 cells (FIG. 8F). It was also found that NKX2-1 did not support KP T3 cell growth (FIG. 8G) as it did in KL LJE1 cells (FIG. 8E). RNA-seq was used to identify the transcriptional targets common to both HDAC3 and NKX2-1 in this KP cell system. Performing the same analysis on RNA-seq data from KP T3 cells (FIG. 8H) as was performed on KL LJE1 cells (FIG. 8D) revealed remarkably little overlap between the target genes co-regulated by HDAC3 and NKX2-1 in KL LJE1 cells and KP T3 cells (FIG. 8I). RNA-seq data from primary GEMM lung tumors (n≥8) revealed that Fgfr1 mRNA was expressed at higher levels in tumors without LKB1 compared to tumors with LKB1 (FIG. 8J), indicating that Fgfr1 expression differences between tumor cells with or without LKB1 is observed across multiple independently-arising tumors.

HDAC3 and NKX2-1 Co-Regulate Target Genes which are Aberrantly Engaged Upon Trametinib Resistance

FGFR1 has been shown to mediate resistance to the FDA-approved MEK inhibitor, trametinib, that acts downstream of Kras to suppress signaling through the Mitogen-Activated Protein Kinase (MAPK) cascade (Ref. 53; incorporated by reference in its entirety). However, therapies directed against Kras effectors activate compensatory pathways that limit their efficacy as single agents, and many current efforts are directed toward elucidating combination therapy approaches that would potentiate clinical benefit from existing Kras effector inhibitors. Using an shRNA screen, FGFR1 was identified as a mediator of trametinib resistance (Ref. 53; incorporated by reference in its entirety). In the KP lung tumor GEMM where trametinib treatment alone is largely ineffective due to rapidly acquired resistance, it was found that co-treatment of trametinib with the FGFR1 inhibitor ponatinib induced tumor regression in this aggressive in vivo model of NSCLC (Ref. 53; incorporated by reference in its entirety).

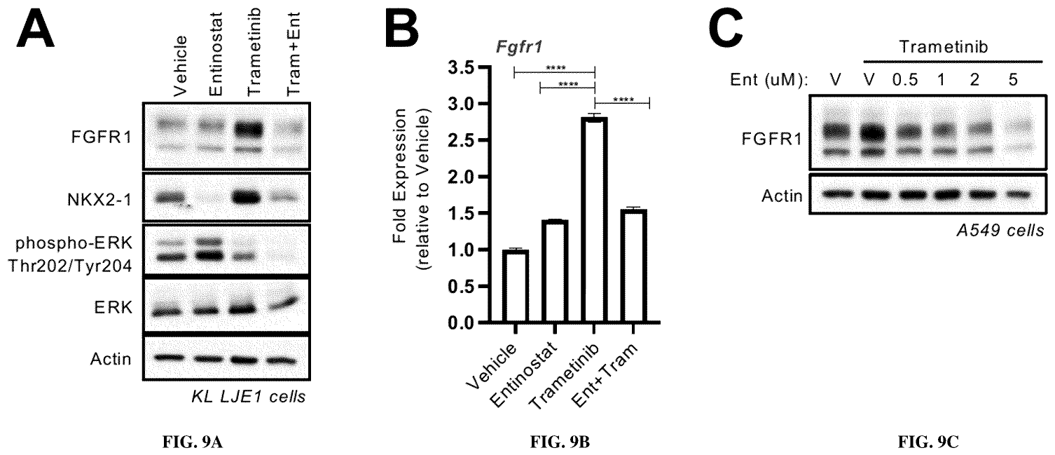

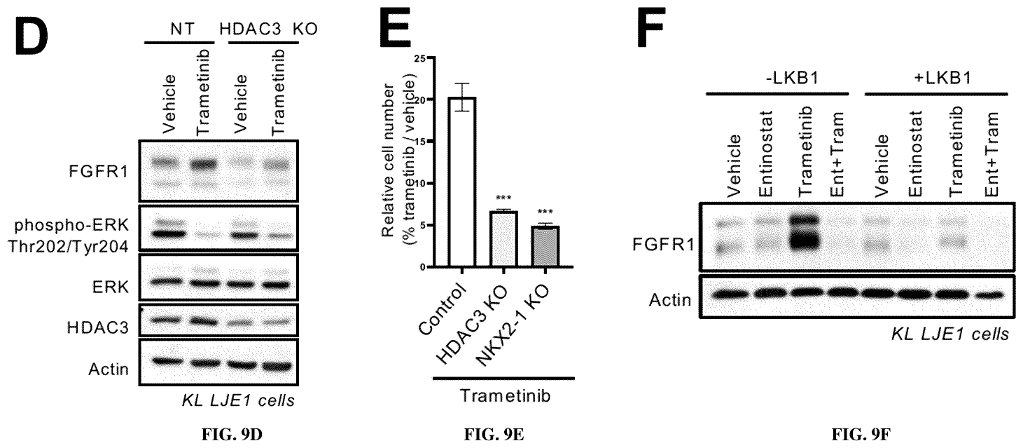

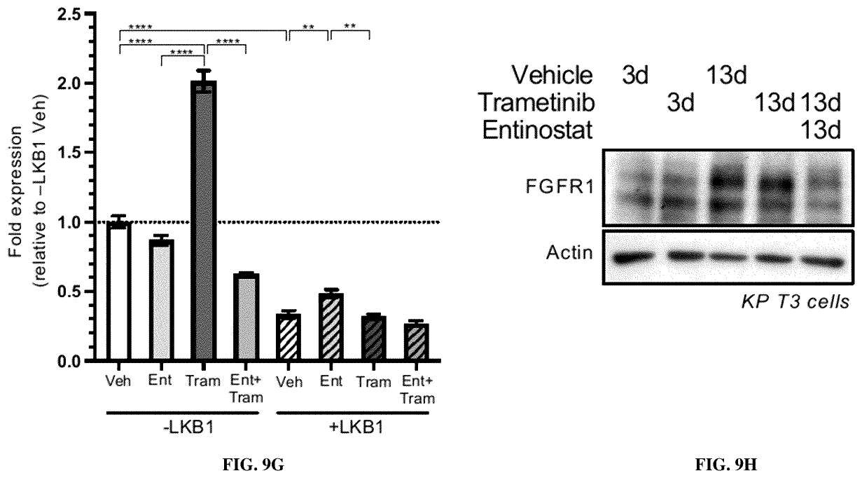

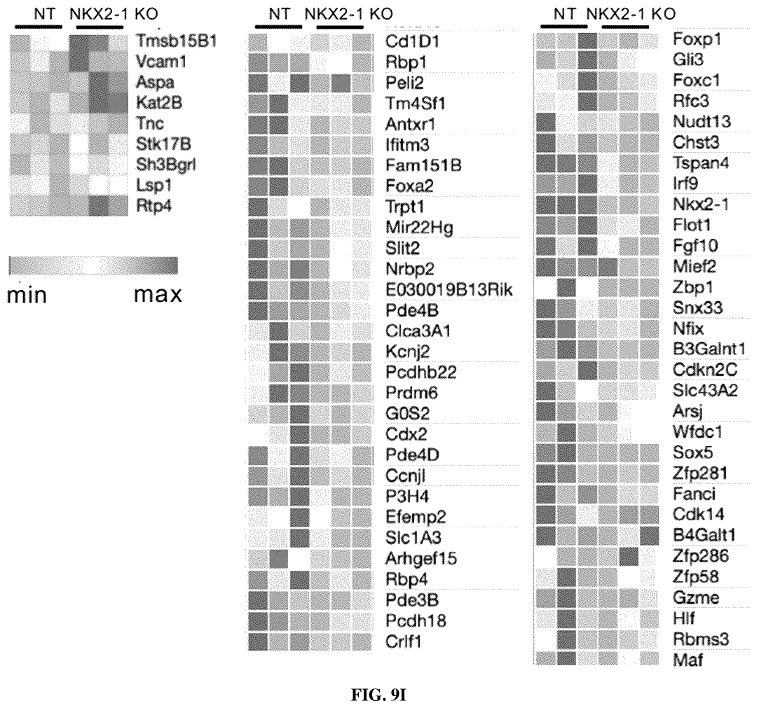

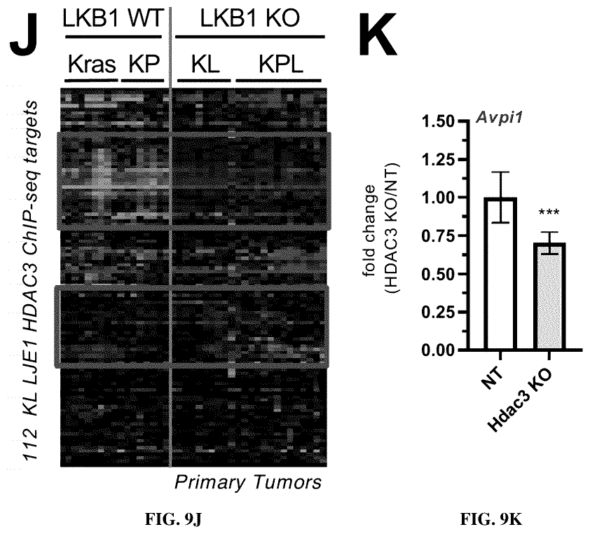

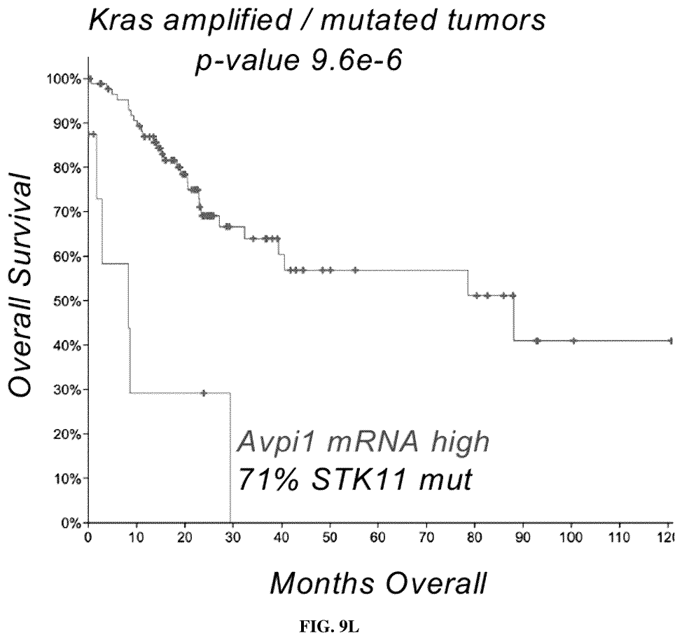

Experiments were conducted during development of embodiments herein to determine whether HDAC inhibition may be an alternative therapeutic approach for blocking trametinib resistance-induced FGFR1 hyperactivation. Short-term (3 day) and long-term (13 day) treatments with trametinib were conducted. Entinostat (MS-275) was selected as the HDAC inhibitor for molecular studies, as it is one of the most selective clinically-tolerated HDAC inhibitors, preferentially inhibiting HDAC1 and HDAC3. Indeed, FGFR1 protein was strongly induced upon 13-day trametinib in a manner that could be reversed by co-treatment with entinostat (FIG. 4A). Moreover, NKX2-1 protein itself was regulated in a similar fashion (FIG. 4A): long-term Trametinib Induced, Entinostat Reversed (“TIER”). This indicated that upregulation of NKX2-1 activity is an HDAC-dependent component of the trametinib resistance response, and FGFR1 behaves as a read-out of this molecular event in KL cells. It was confirmed that neither FGFR1 protein or mRNA levels were impacted by 1 uM entinostat treatment alone (FIG. 9A-B). Blunted FGFR1 induction from trametinib and entinostat co-treatment corresponded with a striking reduction in ERK activity as assessed by phosphorylation at Thr202/Tyr204 (FIG. 9A). Using the A549 cell line, FGFR1 behaved as a TIER gene in human KRAS, LKB1-mutant lung cancer cells (FIG. 9C). Comparing NT versus HDAC3 KO cells revealed that HDAC3 was required for the trametinib-enhanced FGFR1 expression (FIG. 9D). Indeed, both HDAC3 and NKX2-1 were required for maximal cellular resistance to trametinib (FIG. 9E).

Because genes associated with direct HDAC3 genome binding had displayed an LKB1-dependent gene expression pattern in primary tumors (FIG. 2C), experiments were conducted to determine whether FGFR1 expression behaved in an LKB1-dependent manner. LKB1 was reintroduced into KL LJE1 cells and the impact on FGFR1 response to treatment was queried, which revealed that FGFR1 only behaved as a TIER gene in the absence of LKB1 (FIG. 9F-G). Consistently, FGFR1 expression did not follow the TIER pattern in KP cells (FIG. 9H). Together, the implication is that HDAC inhibitors which target HDAC3 such as entinostat may block the induction of a transcriptional program that becomes hyperactivated as Kras, LKB1-mutant lung cancer cells develop resistance to trametinib.

Since NKX2-1 is a transcription factor with multiple target genes, it was next explored whether FGFR1 is part of a larger set of HDAC3-dependent NKX2-1 target genes that become upregulated upon trametinib resistance. To this end, KL LJE1 cells treated as in FIG. 4A were profiled by RNA-seq. To identify genes exhibiting the TIER gene expression pattern, the genes upregulated upon 13 day trametinib compared to vehicle (2,141 genes) were defined, and then plotted their gene expression across all five treatment conditions (FIG. 4B). This analysis identified that 285/2141 (13%) genes displayed the TIER expression pattern. One of these 285 TIER genes was Nkx2-1 itself (FIG. 4C), consistent with the immunoblot results in FIG. 4A. To identify whether a broader set of NKX2-1-dependent genes behaved similarly, the 285 TIER genes were queried against RNA-seq data from NKX2-1 KO cells using GSEA. Indeed, the TIER gene set was negatively enriched in cells deleted for NKX2-1 (FIG. 4D), indicating that a group of TIER genes are NKX2-1-dependent in their basal gene expression pattern as well. TIER gene expression was plotted across RNA-seq data from NT and NKX2-1 KO cells in order to identify these genes (FIG. 9I). Thus, induction of a cassette of NKX2-1-regulated genes is a component of the trametinib resistance transcriptional program that can be reversed by HDAC inhibition. 112 of the 285 (39%) TIER genes were associated with at least one HDAC3 ChIP-seq binding site (FIG. 4E). Moreover, many of these 112 HDAC3-bound TIER genes display LKB1-dependent gene expression patterns in primary tumors (FIG. 9J). Together, identification of the TIER genes revealed a set of direct HDAC3 target genes hyperactivated in KL cells upon trametinib resistance. Trametinib resistance accentuates the necessity for the HDAC3/NKX2-1 pathway in NSCLC.

Avpil provides an example of how a TIER gene behaves across the experimental conditions queried (FIG. 4F-G, 9K-L). Avpil is a TIER gene (FIG. 4F), is directly bound by HDAC3 (FIG. 4G), and its expression is reduced upon deletion of HDAC3 in KL NSCLC cells (FIG. 9K). Thus, Avpil expression in lung tumor cells is directly regulated by HDAC3, induced by trametinib resistance, and reduced by HDAC inhibitor treatment in KL cells. Querying The Cancer Genome Atlas (TCGA) human Lung Adenocarcinoma dataset we found that, in tumors harboring KRAS amplification or mutation at G12, 71% of the tumors with high AVP11 expression harbored STK11 mutation, corresponding with the genetics of the KL cell lines studied. High AVP11 expression within this patient cohort correlated with substantially shorter overall survival (8.48 versus 88.07 median months overall) (FIG. 9L).

### Trametinib Plus Entinostat Treatment Elicits Therapeutic Benefit in the KL GEMM Model

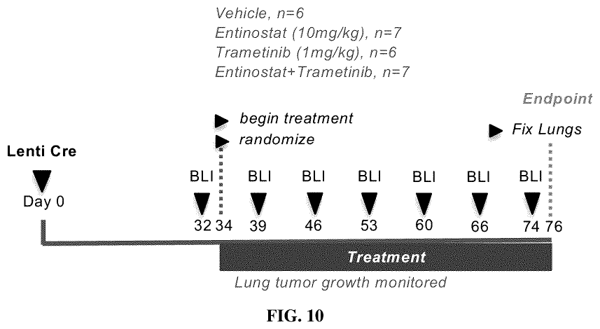

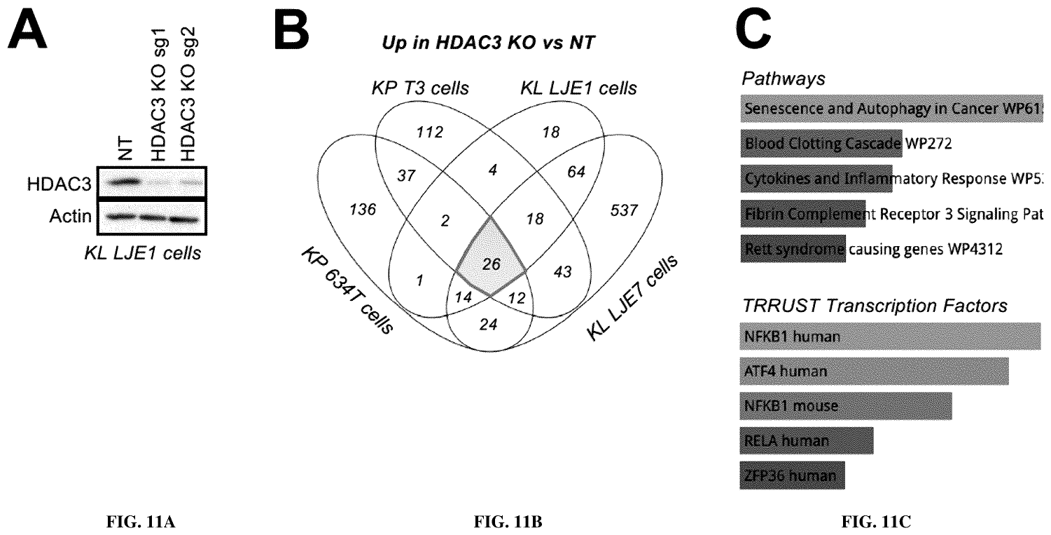

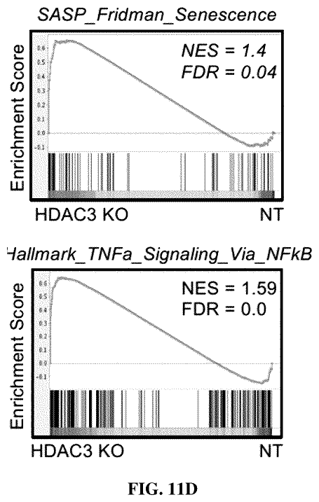

Identification of the TIER genes revealed that trametinib resistance accentuates the necessity for the HDAC3 pathway in a manner that can be reversed by co-treatment with the HDAC inhibitor entinostat in KL NSCLC cells. Thus, to assess therapeutic efficacy of the combination treatment in vivo, experiments were conducted during development of embodiments herein to treat KL mice with entinostat, trametinib, or the combination of entinostat plus trametinib (“Ent+Tram”) (FIG. 10). Lung tumorigenesis was initiated in KL mice (day 0) and tumor growth was monitored with bioluminescence imaging (BLI). 34 days after Cre recombinase (Lenti-Cre) administration, mice were randomized into treatment groups and treatment was initiated. Drug was administered by oral gavage throughout a 42 day treatment course, and material collected at endpoint was used for quantitation of tumor burden. BLI imaging indicated that after 42 days of treatment the Ent+Tram group exhibited the lowest tumor burden of all treatment groups (FIG. 5A). H&E-stained lung sections from the treatment mice at endpoint illustrated that Ent+Tram mice harbored distinctly less tumor burden than all other treatment groups (FIG. 5B). Quantitation of tumor burden from the H&E-stained sections from all mice in the study confirmed that neither entinostat nor trametinib alone impacted tumor burden compared to vehicle control, but the Ent+Tram drug combination elicited significantly reduced tumor burden compared to all other treatment groups (FIG. 5C). The Ent+Tram group contained smaller and fewer tumors than other treatment groups (FIG. 5D-E). This data identified that entinostat and trametinib, which are both clinically viable drugs that do not elicit efficacy as single agent treatments for lung cancer, impart therapeutic efficacy in the KL GEMM model when administered simultaneously.

HDAC3 Target Genes Common to KL and KP Lung Cancer Cells are Associated with the NF-kB and SASP Pathways

HDAC3 was inactivated using CRISPR/Cas9 in NSCLC cell lines derived from the GEMMs, KP T3 cells and KP 634T cells and two cell lines from the KL GEMM, KL LJE1 cells and KL LJE7 cells, which were established from individual primary tumors (FIG. 11A, 17A-C). HDAC3 inactivation reduced cell growth rates across the four cell lines (FIG. 17D), consistent with the tumor-supporting role of HDAC3 in KP and KL NSCLC. These cell lines were profiled by RNA sequencing (RNA-seq) to identify how HDAC3 inactivation impacted gene expression in NSCLC cells. Results from the four cell lines were compared in order to identify genes consistently altered upon HDAC3 loss (>0.5 fold change, adj. p-value<0.05). A core set of 26 genes were upregulated (FIG. 11B) and 12 genes were downregulated (FIG. 17E) upon loss of HDAC3 across KP and KL lung cancer cells. Pathway analysis did not identify a specific pathway associated with the genes downregulated upon HDAC3 loss. In contrast, the genes upregulated upon HDAC3 loss were significantly enriched for the senescence pathway and the NF-kB/p65 transcription factor (FIG. 11C). Gene set enrichment analysis (GSEA) confirmed that the expression of “SASP Fridman Senescence” and “TNFα Signaling via NF-kB” gene sets were enriched upon HDAC3 inactivation (FIG. 11D). Senescence is a cellular process of largely irreversible cell cycle arrest driven by the concerted action of the RB, p53, and NF-kB/p65 pathways. RB and p53 coordinate to arrest cell cycling, and NF-kB/p65 drives the gene expression of a well-defined set of chemokines known as the senescence-associated secretory phenotype (SASP) to direct immune-mediated senescent cell clearance. The SASP is a bioactive transcriptional program that impacts the microenvironment from senescence onset until senescent cell clearance. HDAC3 could be an endogenous upstream repressor of genes associated with the SASP gene signature in Kras mutant lung cancer cells.

Previously published HDAC3 ChIP-seq data (FIG. 17F) was integrated with RNA-seq data from HDAC3 knockout (KO) cells. This analysis identified 468 genes that were associated with direct HDAC3 genome binding and HDAC3-dependent gene expression in KL lung cancer cells (FIG. 11E). Motif enrichment analysis revealed that the HDAC3 ChIP-seq peaks associated with genes upregulated upon HDAC3 loss (red box, FIG. 11E) were enriched for the NF-kB/p65 DNA binding motif (FIG. 11F). This analysis suggested that NF-kB/p65 was the most enriched transcription factor (TF) at target genes that were directly repressed by HDAC3 in lung cancer cells. HDAC3 has been shown to mediate its function by deacetylating histone H3 at lysine 27 (H3K27) and lysine 9 (H3K9), or via mechanisms independent of histone deacetylation. To query which mechanism was involved in lung cancer cells, H3K27ac and H3K9ac ChIP-seq were performed on KL LJE1 cells. At the HDAC3-bound sites associated with upregulated gene expression, no changes in H3K27ac or H3K9ac were detected (FIG. 17G-H). HDAC3 genome binding was observed near the transcription start site (TSS) of SASP genes, but no major changes in H3K27ac or H3K9ac were observed at these genomic regions (FIG. 11G). Therefore, the HDAC3 mechanism of action at these target genes appears to be independent of H3K27 or H3K9 deacetylation.

HDAC3 represses chemokine gene expression in mouse and human NSCLC cells NF-kB/p65-dependent SASP genes have been previously defined and, of these, the expression of Cc120, Cxc15, Cxc11, Cxc12, Cxc13, IL-1a, and Cebpb was increased (>0.5 fold change, adj. p-value<0.05) in response to HDAC3 KO in two different lung cancer cell lines derived from the KL GEMM (FIG. 12A, 18A). These genes were also upregulated upon HDAC3 KO in lung cancer cells derived from the KP GEMM (FIG. 18B). Furthermore, in A549 cells, which are a widely-used human KRAS mutant lung cancer cell line, these genes were also upregulated in response to HDAC3 KO (FIG. 18C).

As described above, entinostat is a clinically well-tolerated HDAC1—and HDAC3-selective inhibitor, and RGFP966 is an HDAC3-selective tool compound. Treatment of mouse KL cells with entinostat or RGFP966 resulted in robust induction of gene expression (FIG. 12B), consistent with results obtained from HDAC3 genetic deletion experiments (FIGS. 12A, 18A-C). In A549 and H460 cells, which are human KRAS mutant lung cancer cells, entinostat treatment also induced the expression of these genes (FIG. 18D-E). The HDAC3-selective inhibitor BRD3308 induced the expression of these genes in human KRAS mutant NSCLC cells as well (FIG. 18F-G). Indicating that in mouse and human KRAS mutant lung cancer cells, endogenous HDAC3 restrained the expression of a set of chemokine genes. Consequently, both genetic deletion and pharmacological inhibition of HDAC3 increased the expression of this chemokine gene set.

The SASP genes, including those identified here as HDAC3-dependent target genes, encode for proteins which function as secreted chemokines. Using a cytokine array ELISA, the protein expression of 112 cytokines in the cell culture media from NT and HDAC3 KO cells was profiled. 32 cytokines were detected in the media, and 11 of these were differentially expressed (fold change >+/−0.5, p-value<0.05) between NT and HDAC3 KO cells (FIG. 12C). Next, results from the ELISA were compared to the RNA-seq to identify which transcriptional changes corresponded to functional differences in secreted proteins (FIG. 12D). Of the HDAC3-dependent chemokine genes identified by RNA-seq, the ELISA confirmed that Cc120, Cxc15, and Cxc12 protein secretion was HDAC3 dependent. An additional group of 7 secreted proteins were also identified by ELISA as HDAC3-dependent. Of these, Cc15, Cxc110, G-Csf, and Gdf-15 were readily detectable at the mRNA level and were upregulated upon HDAC3 deletion (FIG. 18H).

### NF-kB/p65 for HDAC3 Repression of Chemokine Gene Expression in Lung Cancer Cells

CRISPR/Cas9 was used to inactivate p65, and it was confirmed that p65 supports basal expression of Cc120, Cxc15, Cxc11, Cxc12, Cxc13, and IL-1a (FIG. 19A). Cxc110, Cc15, 164 G-csf, and Gdf-15 expression was not significantly altered by p65 inactivation in basal conditions (FIG. 19A).

CRISPR/Cas9 was used to inactivate p65 in HDAC3 KO cells (FIG. 13A). Gene expression analysis confirmed that HDAC3 KO induced the expression of the genes identified in FIG. 12D, and revealed that p65 was required for the HDAC3-dependent effects on Cc120, Cxc15, Cxc11, Cxc12, Cxc13, IL-1a, Cxc110, and Cc15 (FIG. 13B). Notably, across the cell systems, IL-1a mRNA was expressed at extremely low levels, often barely above the threshold of detection. Taken together with the observation that IL-1a protein is not detected in the media (FIG. 12C), it is unlikely that IL-1a is a functionally relevant target in Kras mutant lung cancer cells and, therefore, it was omitted from further analysis. HDAC3-dependent induction of G-csf and Gdf-15 expression did not require p65 (FIG. 3B) and, therefore, these genes were excluded from subsequent analysis of the HDAC3-p65 pathway. Next, RNA-seq was used to identify all genes that were transcriptionally repressed by HDAC3 in a p65-dependent manner (FIG. 13C, 19B). 23 genes were dependent on p65 for their HDAC3-responsive expression pattern (FIG. 13C). This suggested a model where the impact of HDAC3 on p65 function is limited with regard to the number of genes impacted, but potent with regard to the magnitude of the gene expression changes. Furthermore, p65 facilitates maximal gene expression induction in response to both entinostat (FIG. 19C) and BRD3308 (FIG. 19D).

In Kras mutant lung cancer cells HDAC3 robustly repressed a set of p65-dependent genes, Cc120, Cxc15, Cxc1, Cxc12, Cxc13, Cxc110, and Cc15, defined as HDAC3-Repressed Chemokine Genes (“HRCG”). These genes are the focus of subsequent analysis of the HDAC3-p65 pathway. Next, we queried the impact of HDAC3 on NF-kB/p65 protein. HDAC inhibition with entinostat, RGFP966, or the pan-HDAC inhibitor trichostatin a (TSA) increased the quantity of p65 protein in the nuclear fraction (FIG. 13D). Using a commercially-available antibody developed to recognize acetylation of transfected p65 on lysine 310 (K310) and two different published p65 cDNA constructs, entinostat treatment increased K310 acetylation on p65 (FIG. 13E, 19E). To understand the impact of p65 acetylation at K310, K310 was mutated to alanine (K310A), which confirmed that entinostat and BRD3308 induce acetylation of wildtype p65 at K310, and validated that the p65 K310A mutant ablates all acetylation at K310 of p65 (FIG. 19F). Analysis in basal conditions identified that K310 of p65 impacted HRCG expression, although in most cases it elicited only a modest impact on net gene expression compared to wildtype p65 (FIG. 19G). In the presence of entinostat, K310 only significantly altered the gene expression response of Cc120, Cxc15, and Cc15 (FIG. 19H).

### HDAC3 Regulates HRCG Expression in Lung Tumors In Vivo

The KL and KP GEMMs recapitulate two major molecular subtypes of KRAS mutant NSCLC. These GEMMs were previously crossed to the HDAC3 floxed mouse model to generate the KL-HDAC3 and KP-HDAC3 GEMMs. When lentivirus expressing Cre recombinase was delivered by intratracheal intubation to the lungs of these mice, tumorigenesis was initiated in infected lung cells and, in KL-HDAC3 and KP-HDAC3 mice, HDAC3 was genetically deleted selectively within lung tumor cells at the time of tumor initiation. These GEMMs facilitated genetic assessment of HDAC3 function in vivo within autochthonous lung tumor cells in the context of an otherwise HDAC3-wildtype and immune-competent animal. Here, to assess HRCG expression in lung tumors in vivo, immunohistochemistry (IHC) was applied to lung sections from tumor-bearing mice from the KL-HDAC3 and KP-HDAC3 GEMMs. A commercially available antibody directed against Cxc15 had been used on FFPE GEMM lung tumors and, this antibody was selected to query HRCG expression by IHC. IHC on FFPE lung tissue from the KL-HDAC3 GEMM revealed that Cxc15 protein expression was elevated in KL-HDAC3 tumors as compared to KL lung tumors from littermate control mice (FIG. 14A-B). Cxc15 protein expression was also elevated in KP-HDAC3 lung tumors compared to KP lung tumors (FIG. 20A-B). This data confirmed that genetic inactivation of HDAC3 resulted in de-repression of HRCG expression in Kras mutant lung tumors in vivo in both the KL and KP GEMMs, consistent with findings from cell culture experiments where HDAC3 inactivation induced HRCG expression in KL and KP lung cancer cell lines.

Next, pharmacological inhibition of HDAC3 was assessed for impacting HRCG expression in vivo as observed in cell culture experiments (FIG. 12B, 18D-G). Lung tumorigenesis was initiated in KL GEMM mice, and disease growth was monitored over time using bioluminescence imaging (BLI) potentiated by the Rosa26^{LSL-luferase} genetic allele. When tumor burden was established in all mice (BLI >5×10⁷ photons/sec), mice were randomized and treatment was initiated. Mice were treated daily for 5 days with vehicle or 10 mg/kg/day entinostat administered by oral gavage, after which lung tumors and liver were collected (FIG. 14C). Using qRT-PCR, gene expression of HDAC3 target genes in lung tumors and liver was assessed. In lung tumors, entinostat treatment potently induced HRCG expression (FIG. 14D). However, in livers from the same mice, entinostat treatment did not induce HRCG expression (FIG. 14E). To confirm that entinostat treatment was engaging on-target effects in the liver samples, the gene expression of canonical HDAC3 liver target genes Nr1d1 and Arntl was assessed (FIG. 14E). Indeed, entinostat treatment impacted Nr1d1 and Arntl gene expression in the liver. This suggested that, despite systemic administration, entinostat treatment induced HRCG expression selectively in lung tumors but not in livers from the same mice.

The recently-developed Kras^{G12D}-selective inhibitor MRTX1133 was used as a tool to inactivate Kras^{G12D} in lung cancer cells derived from the KL GEMM. First, it was confirmed that MRTX1133 treatment resulted in on-target Kras inhibition by assessing ERK phosphorylation at Thr202/Tyr204 (FIG. 20C), which is an established downstream read-out of Kras pathway signaling. Next, qRT-PCR was used to assess HRCG expression. HDAC3 KO induced HRCG expression (FIG. 14F, 20D). Remarkably, however, while MRTX1133 treatment alone did not impact the expression of these genes, MRTX1133 treatment reversed the HDAC3-dependent induction of Cxc15, Cxc1, Cxc12, and Cxc13 expression (FIG. 14F). This suggested that oncogenic Kras^{G12D} facilitated HDAC3 engagement with these genes and, by extension, that HDAC3 repression of HRCG may be a tumor-specific molecular event. In contrast, the expression of Cc15 and Cxc110 did not follow this pattern, as their expression was maintained in the presence of MRTX1133 (FIG. 20D). Cxc15, Cxc1, Cxc12, and Cxc13 are considered immunosuppressive chemokines in the lung, whereas Cc15 and Cxc110 are known to mediate antitumor immunity.

Thus, these findings revealed a divergence in response between tumorigenic chemokines and chemokines involved in tumor growth control, respectively. This was tested using a different therapeutic agent for inhibiting Kras pathway activity. Since MEK is a key downstream effector of Kras pathway signaling, the well-characterized MEK inhibitor trametinib was used. Comparable to findings with MRTX1133, trametinib treatment reversed the induction of HRCG expression in response to HDAC3 inactivation for immunosuppressive HRCGs, but not for immunosurveillance-related HRCGs (FIG. 20E).

HDAC3 Genetic Deletion Results in T-Cell Recruitment into GEMM Lung Tumors

Assessment of H&E-stained lung sections from KL-HDAC3 mice revealed a striking influx of immune cells in HDAC3-deleted lung tumors (FIG. 15A). The SASP has been shown to recruit immune cells including NK cells and T-cells to tumors. IHC was used to visualize macrophages, NK cells, neutrophils, and T-cells in KL and KL-HDAC3 lung tumors (FIG. 15B, 21A). No differences in macrophage, NK cell, or neutrophil influx in response to HDAC3 deletion were observed (FIG. 21A). However, the quantity of CD3+ T-cells was markedly increased in HDAC3 deleted lung tumors (FIG. 15B-C). A similar phenotype was observed in the KP-HDAC3 GEMM, where HDAC3 deleted tumors exhibited a clear influx of immune cells (FIG. 15D). HDAC3 deletion did not impact the quantity of macrophages, NK cells, or neutrophils (FIG. 21B), but the quantity of T-cells was significantly higher in KP lung tumors deleted for HDAC3 (FIG. 15E-F). Thus, deletion of HDAC3 selectively in lung tumors in vivo resulted in increased T-cell recruitment and infiltration into KL and KP lung tumors. In Kras mutant lung tumors endogenous HDAC3 restrains T-cell infiltration.

Cc120, Cxc15, Cxc12, Cc15, and Cxc110 corresponded to functional differences in proteins secreted from Kras mutant lung cancer cells (FIG. 12D). Cxc19, Cxc110, and Cxc11 are known to recruit T-cells. In the lung cancer cells, neither Cxc19 nor Cxc11 were detected at the mRNA or protein level, but Cxc110 mRNA was readily detectable. To query the molecular link between HDAC3 and Cxc110, the Cxc110 response to HDAC3 modulation was assessed. Cxc110 was identified as a p65-dependent HRCG (FIG. 13D) and an HDAC3-dependent secreted protein (FIG. 12D). In vivo, Cxc110 expression was induced upon entinostat treatment selectively in lung tumor cells (FIG. 14D-E). Examination of ChIP-seq data at the Cxc110 genomic locus identified HDAC3 binding near the Cxc110 TSS in lung cancer cells (FIG. 15G). Consistent with findings at other HRCGs (FIG. 11G), no changes in H3K27 or H3K9 acetylation at the HDAC3-bound region of the Cxc110 genomic locus were observed (FIG. 15G). HDAC3 inactivation via both genetic deletion and pharmacological inhibition induced the expression of Cxc110 in mouse (FIG. 15H-I) and human (FIG. 15J-K, 21C-D) Kras mutant lung cancer cells. Together, this identified Cxc110 as a direct target of HDAC3 transcriptional repression in Kras mutant lung cancer cells.

C—X-C Motif Chemokine Receptor 3 (CXCR3) is a receptor with selectivity for Cxc19, Cxc110, and Cxc111 and, therefore, antibodies directed against CXCR3 were used to test the requirement for the Cxc110-CXCR3 axis in vivo. Tumors were initiated in KL-HDAC3 and KP-HDAC3 mice, tumor growth was tracked over time using bioluminescence imaging, mice were randomized into two treatment groups, mice were treated with either anti-CXCR3 blocking antibody or IgG control, and tumor-bearing lungs were collected at endpoint (FIG. 23). CXCR3 blocking antibody treatment significantly reduced the quantity of T-cells within HDAC3-deleted tumors in the KL-HDAC3 (FIG. 23, bottom left) and KP-HDAC3 (FIG. 23 bottom right) GEMMs. These findings suggest that Cxc110 contributes to the T-cell recruitment elicited by HDAC3 inactivation in vivo.

Entinostat Plus Trametinib Combination Therapy Enhances T-Cell Infiltration into GEMM Lung Tumors

HDAC3 endogenously restrains T-cell infiltration into Kras mutant lung tumors, and that expression of the HRCGs associated with this phenotype are induced by treatment with HDAC3-targeting inhibitors including entinostat. The implication is that pharmacological agents which target HDAC3 could provide a therapeutic means for recruiting T-cells into KRAS mutant lung tumors, which would be clinically advantageous. However, if T-cell recruitment occurred concurrently with the induction of an immunosuppressive chemokine program, therapeutic benefit could be occluded. Findings with MRTX1133 and trametinib treatment suggested that inhibition of the Kras pathway may dissociate immunosuppressive and immunosurveillance-related HRCG expression (FIG. 14F, 20D-E). Indeed, in KRAS mutant lung cancer cells, combination treatment of trametinib with entinostat blunted the induction of Cxc15, Cxc11, and Cxc13 in response to entinostat, whereas trametinib and entinostat co-treatment enhanced the expression of Cc15 and Cxc110 (FIG. 16A). Thus, co-treatment of entinostat with trametinib may maximize potential T-cell recruitment.

Trametinib and entinostat combination treatment elicited therapeutic benefit in the KL GEMM of NSCLC. The pharmacological inhibition of HDAC3 was assessed in the context of trametinib treatment impacting T-cell recruitment into lung tumors. To this end, lung tumorigenesis was initiated in the KL GEMM, and disease growth was monitored over time by bioluminescence imaging. When tumor burden was established in all mice, mice were randomized and treatment was initiated. Mice were treated daily by oral gavage with vehicle, 5 mg/kg entinostat, 1 mg/kg trametinib, or entinostat+trametinib (Ent+Tram) for 3 weeks, after which lungs were collected. H&E-stained lung sections were prepared from FFPE tissue and analyzed. Interestingly, Ent+Tram treated tumors exhibited an increased quantity of intratumoral immune cells (FIG. 16B, 22A). RNA-seq performed on treated tumors revealed that the genes selectively upregulated in Ent+Tram treated tumors compared to all other treatment conditions were most associated with T-cells (FIG. 22B). Next, IHC was used to visualize T-cells in the FFPE lung sections. This revealed that Ent+Tram treated lung tumors exhibited an increased quantity of CD3+ T-cells compared to all other treatment conditions (FIG. 16C-D). The important implication is that pharmacological inhibition of HDAC3 in combination with trametinib may provide a therapeutic approach for recruiting T-cells into KL lung tumors.

Flow cytometry was used to assess how trametinib and entinostat treatment affected the tumor immune infiltrate and spleen from treated mice. Ent+Tram combination treatment did not strongly impact Treg frequency (FIG. 22C) or CD4+ or CD8+ T-cell proliferation rates (FIG. 22D). The percentages of naive, effector, and memory CD8+ T-cells were also not potently altered upon Ent+Tram treatment (FIG. 22E). However, the percentage of transitory (TrnsEx) and terminally exhausted (TerEx) CD8+PD-1+ T-cells was reduced to similar degrees in both trametinib and Ent+Tram treated tumors (FIG. 22E). Furthermore, CD8+ T-cells exhibited increased cytokine production capacity specifically in lung tumors from trametinib and Ent+Tram conditions (FIG. 22F). No changes were observed in gMDSCs in response to entinostat treatment alone, however, increased gMDSCs in lung tumors treated with trametinib alone was observed (FIG. 22G). In contrast, Ent+Tram treatment reversed the effect of trametinib on gMDSC influx (FIG. 22G), suggesting that gMDSC recruitment was unlikely to significantly counteract the immunosurveillance function of T-cells recruited into lung tumors in response to Ent+Tram treatment. Systemic Ent+Tram treatment did not reduce the functionality of T-cells recruited into KL lung tumors.

KP T3 cells were injected into the flanks of syngeneic mice, and tumor size was measured by caliper three times per week. 10 days post-injection, mice were randomized into two treatment groups, and T-cell depleting anti-CD4/8a antibodies or IgG2b control antibody was administered weekly by i.p. injection. 13 days post-injection, mice in each treatment group were randomized into two groups, and Ent+Tram treatment was delivered daily by oral gavage.

Ent+Tram treatment potently reduced tumor growth in the IgG2b control antibody group, but tumor size continued to increase in mice treated with anti-CD4/8a antibody (FIG. 16E). Anti-CD4/8a treatment resulted in reduced T-cells within flank tumors from treated mice (FIG. 22H-22I). To test the role of T-cells in tumor growth control in LKB1-null lung cancer cells, LKB1 was inactivated in KP T3 cells using CRISPR/Cas9 (FIG. 22K). Following a similar experimental design as in FIG. 16E, these cells were injected into the flanks of syngeneic mice, treated mice weekly with anti-CD4/8a antibody or IgG2b control, and subsequently administered Ent+Tram combination treatment by daily oral gavage. In Kras mutant lung cancer cells lacking LKB1, Ent+Tram treatment reduced tumor growth in the IgG2b control antibody group, whereas tumor size increased in mice treated with anti-CD4/8a antibody (FIG. 16F). Taken together, this suggested that T-cells contribute to Kras mutant lung tumor growth control in Ent+Tram treated mice.

### REFERENCES

The following references, some of which are cited above by number (e.g., “Ref 1”) are herein incorporated by reference in their entireties.
1. J. E. Bradner, D. Hnisz, R. A. Young, Transcriptional Addiction in Cancer. Cell 168, 629-643 (2017).
2. T. De Raedt et al., PRC2 loss amplifies Ras-driven transcription and confers sensitivity to BRD4-based therapies. Nature 514, 247-251 (2014).
3. J. Shi, C. R. Vakoc, The mechanisms behind the therapeutic activity of BET bromodomain inhibition. Mol Cell 54, 728-736 (2014).
4. N. Tasdemir et al., BRD4 Connects Enhancer Remodeling to Senescence Immune Surveillance. Cancer Discov 6, 612-629 (2016).
5. T. Shimamura et al., Efficacy of BET bromodomain inhibition in Kras-mutant non-small cell lung cancer. Clin Cancer Res 19, 6183-6192 (2013).
6. S. L. Guerra et al., A Deregulated HOX Gene Axis Confers an Epigenetic Vulnerability in KRAS-Mutant Lung Cancers. Cancer Cell 37, 705-719 e706 (2020).
7. R. W. Johnstone, J. D. Licht, Histone deacetylase inhibitors in cancer therapy: is transcription the primary target? Cancer Cell 4, 13-18 (2003).
8. A. C. West, R. W. Johnstone, New and emerging HDAC inhibitors for cancer treatment.

### J Clin Invest 124, 30-39 (2014).

9. E. Ceccacci, S. Minucci, Inhibition of histone deacetylases in cancer therapy: lessons from leukaemia. Br J Cancer 114, 605-611 (2016).
10. Z. Lu et al., Epigenetic therapy inhibits metastases by disrupting premetastatic niches.

### Nature 579, 284-290 (2020).

11. M. J. Topper et al., Epigenetic Therapy Ties MYC Depletion to Reversing Immune Evasion and Treating Lung Cancer. Cell 171, 1284-1300 e1221 (2017).
12. D. O. Adeegbe et al., Synergistic Immunostimulatory Effects and Therapeutic Benefit of Combined Histone Deacetylase and Bromodomain Inhibition in Non-Small Cell Lung Cancer. Cancer Discov 7, 852-867 (2017).
13. C. F. Malone et al., mTOR and HDAC Inhibitors Converge on the TXNIP/Thioredoxin Pathway to Cause Catastrophic Oxidative Stress and Regression of RAS-Driven Tumors.

### Cancer Discov 7, 1450-1463 (2017).

14. D. Morel, D. Jeffery, S. Aspeslagh, G. Almouzni, S. Postel-Vinay, Combining epigenetic drugs with other therapies for solid tumours—past lessons and future promise. Nat Rev Clin Oncol 17, 91-107 (2020).
15. L. Ny et al., The PEMDAC phase 2 study of pembrolizumab and entinostat in patients with metastatic uveal melanoma. Nat Commun 12, 5155 (2021).
16. A. S. Truong et al., Entinostat induces antitumor immune responses through immune editing of tumor neoantigens. J Clin Invest 131, (2021).
17. B. J. Christmas et al., Entinostat Converts Immune-Resistant Breast and Pancreatic Cancers into Checkpoint-Responsive Tumors by Reprogramming Tumor-Infiltrating MDSCs. Cancer Immunol Res 6, 1561-1577 (2018).
18. A. Orillion et al., Entinostat Neutralizes Myeloid-Derived Suppressor Cells and Enhances the Antitumor Effect of PD-1 Inhibition in Murine Models of Lung and Renal Cell Carcinoma. Clin Cancer Res 23, 5187-5201 (2017).
19. M. J. Emmett, M. A. Lazar, Integrative regulation of physiology by histone deacetylase 3.

### Nat Rev Mol Cell Biol 20, 102-115 (2019).

20. M. J. Emmett et al., Histone deacetylase 3 prepares brown adipose tissue for acute thermogenic challenge. Nature 546, 544-548 (2017).
21. Z. Sun et al., Hepatic Hdac3 promotes gluconeogenesis by repressing lipid synthesis and sequestration. Nat Med 18, 934-942 (2012).
22. Z. Sun et al., Deacetylase-independent function of HDAC3 in transcription and metabolism requires nuclear receptor corepressor. Mol Cell 52, 769-782 (2013).
23. S. K. Knutson et al., Liver-specific deletion of histone deacetylase 3 disrupts metabolic transcriptional networks. EMBO J 27, 1017-1028 (2008).
24. L. Ding et al., Somatic mutations affect key pathways in lung adenocarcinoma. Nature 455, 1069-1075 (2008).
25. M. Sanchez-Cespedes et al., Inactivation of LKB1/STK11 is a common event in adenocarcinomas of the lung. Cancer Res 62, 3659-3662 (2002).
26. J. D. Campbell et al., Distinct patterns of somatic genome alterations in lung adenocarcinomas and squamous cell carcinomas. Nat Genet 48, 607-616 (2016).
27. F. Skoulidis, J. V. Heymach, Co-occurring genomic alterations in non-small-cell lung cancer biology and therapy. Nat Rev Cancer 19, 495-509 (2019).
28. J. M. Lizcano et al., LKB1 is a master kinase that activates 13 kinases of the AMPK subfamily, including MARK/PAR-1. EMBO J 23, 833-843 (2004).
29. L. J. Eichner et al., Genetic Analysis Reveals AMPK Is Required to Support Tumor Growth in Murine Kras-Dependent Lung Cancer Models. Cell Metab 29, 285-302 e287 (2019).
30. C. W. Murray et al., An LKB1-SIK Axis Suppresses Lung Tumor Growth and Controls Differentiation. Cancer Discov 9, 1590-1605 (2019).
31. P. E. Hollstein et al., The AMPK-Related Kinases SIK1 and SIK3 Mediate Key Tumor-Suppressive Effects of LKB1 in NSCLC. Cancer Discov 9, 1606-1627 (2019).
32. N. J. Darling, P. Cohen, Nuts and bolts of the salt-inducible kinases (SIKs). Biochem J 478, 1377-1397 (2021).
33. M. Martin, R. Kettmann, F. Dequiedt, Class IIa histone deacetylases: regulating the regulators. Oncogene 26, 5450-5467 (2007).
34. M. M. Mihaylova, R. J. Shaw, Metabolic reprogramming by class I and II histone deacetylases. Trends Endocrinol Metab 24, 48-57 (2013).
35. R. U. Svensson et al., Inhibition of acetyl-CoA carboxylase suppresses fatty acid synthesis and tumor growth of non-small-cell lung cancer in preclinical models. Nat Med 22, 1108-1119 (2016).
36. D. B. Shackelford et al., LKB1 inactivation dictates therapeutic response of non-small cell lung cancer to the metabolism drug phenformin. Cancer Cell 23, 143-158 (2013).
37. J. M. Kaufman et al., LKB1 Loss induces characteristic patterns of gene expression in human tumors associated with NRF2 activation and attenuation of PI3K-AKT. J Thorac Oncol 9, 794-804 (2014).
38. L. Chen et al., A Sensitive NanoString-Based Assay to Score STK11 (LKB1) Pathway Disruption in Lung Adenocarcinoma. J Thorac Oncol 11, 838-849 (2016).
39. M. G. Guenther, O. Barak, M. A. Lazar, The SMRT and N-CoR corepressors are activating cofactors for histone deacetylase 3. Mol Cell Biol 21, 6091-6101 (2001).
40. H. Tanaka et al., Lineage-specific dependency of lung adenocarcinomas on the lung development regulator TTF-1. Cancer Res 67, 6007-6011 (2007).
41. T. Yamaguchi, Y. Hosono, K. Yanagisawa, T. Takahashi, NKX2-1/TTF-1: an enigmatic oncogene that functions as a double-edged sword for cancer cell survival and progression.

### Cancer Cell 23, 718-723 (2013).

42. H. Watanabe et al., Integrated cistromic and expression analysis of amplified NKX2-1 in lung adenocarcinoma identifies LMO3 as a functional transcriptional target. Genes Dev 27, 197-210 (2013).
43. K. A. Kwei et al., Genomic profiling identifies TITF1 as a lineage-specific oncogene amplified in lung cancer. Oncogene 27, 3635-3640 (2008).
44. E. L. Snyder et al., Nkx2-1 represses a latent gastric differentiation program in lung adenocarcinoma. Mol Cell 50, 185-199 (2013).
45. G. Mollaoglu et al., The Lineage-Defining Transcription Factors SOX2 and NKX2-1 Determine Lung Cancer Cell Fate and Shape the Tumor Immune Microenvironment. Immunity 49, 764-779 e769 (2018).
46. M. M. Winslow et al., Suppression of lung adenocarcinoma progression by Nkx2-1.

### Nature 473, 101-104 (2011).

47. L. A. Garraway, W. R. Sellers, Lineage dependency and lineage-survival oncogenes in human cancer. Nat Rev Cancer 6, 593-602 (2006).
48. R. L. Philips et al., HDAC3 restrains CD8-lineage genes to maintain a bi-potential state in CD4(+)CD8(+) thymocytes for CD4-lineage commitment. Elife 8, (2019).
49. L. Zhang et al., Hdac3 Interaction with p300 Histone Acetyltransferase Regulates the Oligodendrocyte and Astrocyte Lineage Fate Switch. Dev Cell 36, 316-330 (2016).
50. V. Gocheva et al., Quantitative proteomics identify Tenascin-C as a promoter of lung cancer progression and contributor to a signature prognostic of patient survival. Proc Natl Acad Sci USA 114, E5625-E5634 (2017).
51. S. Dai, Z. Zhou, Z. Chen, G. Xu, Y. Chen, Fibroblast Growth Factor Receptors (FGFRs): Structures and Small Molecule Inhibitors. Cells 8, (2019).
52. I. S. Babina, N. C. Turner, Advances and challenges in targeting FGFR signalling in cancer. Nat Rev Cancer 17, 318-332 (2017).
53. E. Manchado et al., A combinatorial strategy for treating KRAS-mutant lung cancer.

### Nature 534, 647-651 (2016).

54. K. J. Falkenberg, R. W. Johnstone, Histone deacetylases and their inhibitors in cancer, neurological diseases and immune disorders. Nat Rev Drug Discov 13, 673-691 (2014).
55. L. Venturutti et al., TBL1XR1 Mutations Drive Extranodal Lymphoma by Inducing a Pro-tumorigenic Memory Fate. Cell 182, 297-316 e227 (2020).
56. Y. Jiang et al., CREBBP Inactivation Promotes the Development of HDAC3-Dependent Lymphomas. Cancer Discov 7, 38-53 (2017).
57. P. Mondello et al., Selective Inhibition of HDAC3 Targets Synthetic Vulnerabilities and Activates Immune Surveillance in Lymphoma. Cancer Discov 10, 440-459 (2020).
58. M. P. Phelps, J. N. Bailey, T. Vleeshouwer-Neumann, E. Y. Chen, CRISPR screen identifies the NCOR/HDAC3 complex as a major suppressor of differentiation in rhabdomyosarcoma. Proc Natl Acad Sci USA 113, 15090-15095 (2016).
59. O. Maertens et al., MAPK Pathway Suppression Unmasks Latent DNA Repair Defects and Confers a Chemical Synthetic Vulnerability in BRAF-, NRAS-, and NF1-Mutant Melanomas. Cancer Discov 9, 526-545 (2019).
60. Z. Wang et al., SETD5-Coordinated Chromatin Reprogramming Regulates Adaptive Resistance to Targeted Pancreatic Cancer Therapy. Cancer Cell 37, 834-849 e813 (2020).
61. S. Bhaskara et al., Hdac3 is essential for the maintenance of chromatin structure and genome stability. Cancer Cell 18, 436-447 (2010).
62. R. P. Harvey, NK-2 homeobox genes and heart development. Dev Biol 178, 203-216 (1996).
63. S. Guazzi et al., Thyroid nuclear factor 1 (TTF-1) contains a homeodomain and displays a novel DNA binding specificity. EMBO J 9, 3631-3639 (1990).
64. D. Lazzaro, M. Price, M. de Felice, R. Di Lauro, The transcription factor TTF-1 is expressed at the onset of thyroid and lung morphogenesis and in restricted regions of the foetal brain. Development 113, 1093-1104 (1991).
65. M. N. Stanfel, K. A. Moses, R. J. Schwartz, W. E. Zimmer, Regulation of organ development by the NKX-homeodomain factors: an NKX code. Cell Mol Biol (Noisy-le-grand) Suppl 51, OL785-799 (2005).
66. L. P. Fernandez, A. Lopez-Marquez, P. Santisteban, Thyroid transcription factors in development, differentiation and disease. Nat Rev Endocrinol 11, 29-42 (2015).
67. V. K. Anagnostou, K. N. Syrigos, G. Bepler, R. J. Homer, D. L. Rimm, Thyroid transcription factor 1 is an independent prognostic factor for patients with stage I lung adenocarcinoma. J Clin Oncol 27, 271-278 (2009).
68. Y. Maeda et al., Kras(G12D) and Nkx2-1 haploinsufficiency induce mucinous adenocarcinoma of the lung. J Clin Invest 122, 4388-4400 (2012).
69. A. F. M. Dost et al., Organoids Model Transcriptional Hallmarks of Oncogenic KRAS Activation in Lung Epithelial Progenitor Cells. Cell Stem Cell 27, 663-678 e668 (2020).
70. R. Zewdu et al., An NKX2-1/ERK/WNT feedback loop modulates gastric identity and response to targeted therapy in lung adenocarcinoma. Elife 10, (2021).
71. L. Wang et al., An Acquired Vulnerability of Drug-Resistant Melanoma with Therapeutic Potential. Cell 173, 1413-1425 e1414 (2018).
72. F. Faiao-Flores et al., HDAC Inhibition Enhances the In Vivo Efficacy of MEK Inhibitor Therapy in Uveal Melanoma. Clin Cancer Res 25, 5686-5701 (2019).
73. A. Tanimoto et al., Histone Deacetylase 3 Inhibition Overcomes BIM Deletion Polymorphism-Mediated Osimertinib Resistance in EGFR-Mutant Lung Cancer. Clin Cancer Res 23, 3139-3149 (2017).
74. F. J. Sanchez-Rivera et al., Rapid modelling of cooperating genetic events in cancer through somatic genome editing. Nature 516, 428-431 (2014).
75. M. DuPage, A. L. Dooley, T. Jacks, Conditional mouse lung cancer models using adenoviral or lentiviral delivery of Cre recombinase. Nat Protoc 4, 1064-1072 (2009).
76. J. Abraham et al., Lineage of origin in rhabdomyosarcoma informs pharmacological response. Genes Dev 28, 1578-1591 (2014).
77. L. Shen et al., Class I histone deacetylase inhibitor entinostat suppresses regulatory T cells and enhances immunotherapies in renal and prostate cancer models. PLoS One 7, e30815 (2012).
78. R. T. Kurmasheva et al., Evaluation of entinostat alone and in combination with standard-of-care cytotoxic agents against rhabdomyosarcoma xenograft models. Pediatr Blood Cancer 66, e27820 (2019).
79. M. Ruscetti et al., NK cell-mediated cytotoxicity contributes to tumor control by a cytostatic drug combination. Science 362, 1416-1422 (2018).
80. P. Bankhead et al., QuPath: Open source software for digital pathology image analysis. Sci Rep 7, 16878 (2017).
81. A. Dobin et al., STAR: ultrafast universal RNA-seq aligner. Bioinformatics 29, 15-21 (2013).
82. S. Heinz et al., Simple combinations of lineage-determining transcription factors prime cis-regulatory elements required for macrophage and B cell identities. Mol Cell 38, 576-589 (2010).
83. M. J. de Hoon, S. Imoto, J. Nolan, S. Miyano, Open source clustering software. Bioinformatics 20, 1453-1454 (2004).
84. A. J. Saldanha, Java Treeview--extensible visualization of microarray data. Bioinformatics 20, 3246-3248 (2004).
85. E. Cerami et al., The cBio cancer genomics portal: an open platform for exploring multidimensional cancer genomics data. Cancer Discov 2, 401-404 (2012).
86. J. Gao et al., Integrative analysis of complex cancer genomics and clinical profiles using the cBioPortal. Sci Signal 6, p11 (2013).
87. F. Skoulidis et al., Co-occurring genomic alterations define major subsets of KRAS-mutant lung adenocarcinoma with distinct biology, immune profiles, and therapeutic vulnerabilities. Cancer Discov 5, 860-877 (2015).
88. R. Salgia, R. Pharaon, I. Mambetsariev, A. Nam, M. Sattler, The improbable targeted therapy: KRAS as an emerging target in non-small cell lung cancer (NSCLC). Cell Rep Med 2, 100186 (2021).
89. J. Yang, C. Song, X. Zhan, The role of protein acetylation in carcinogenesis and targeted drug discovery. Front Endocrinol (Lausanne) 13, 972312 (2022).
90. T. Ishizuka, M. A. Lazar, The N-CoR/histone deacetylase 3 complex is required for repression by thyroid hormone receptor. Mol Cell Biol 23, 5122-5131 (2003).
91. D. Feng et al., A circadian rhythm orchestrated by histone deacetylase 3 controls hepatic lipid metabolism. Science 331, 1315-1319 (2011).
92. Y. Liu et al., Metabolic and functional genomic studies identify deoxythymidylate kinase as a target in LKB1-mutant lung cancer. Cancer Discov 3, 870-879 (2013).
93. Y. Chien et al., Control of the senescence-associated secretory phenotype by NF-kappaB promotes senescence and enhances chemosensitivity. Genes Dev 25, 2125-2136 (2011).
94. J. Campisi, Cellular Senescence and Lung Function during Aging. Yin and Yang. Ann Am Thorac Soc 13 Suppl 5, S402-S406 (2016).
95. A. S. L. Chan, M. Narita, Short-term gain, long-term pain: the senescence life cycle and cancer. Genes Dev 33, 127-143 (2019).
96. T. Kuilman, C. Michaloglou, W. J. Mooi, D. S. Peeper, The essence of senescence. Genes Dev 24, 2463-2479 (2010).
97. D. A. Yardley et al., Randomized phase II, double-blind, placebo-controlled study of exemestane with or without entinostat in postmenopausal women with locally recurrent or metastatic estrogen receptor-positive breast cancer progressing on treatment with a nonsteroidal aromatase inhibitor. J Clin Oncol 31, 2128-2135 (2013).
98. Q. C. Ryan et al., Phase I and pharmacokinetic study of MS-275, a histone deacetylase inhibitor, in patients with advanced and refractory solid tumors or lymphoma. J Clin Oncol 23, 3912-3922 (2005).
99. M. Malvaez et al., HDAC3-selective inhibitor enhances extinction of cocaine-seeking behavior in a persistent manner. Proc Nat Acad Sci USA 110, 2647-2652 (2013).
100. K. M. Barton et al., Selective HDAC inhibition for the disruption of latent HIV-1 infection. PLoS One 9, e102684 (2014).
101. L. Chen, W. Fischle, E. Verdin, W. C. Greene, Duration of nuclear NF-kappaB action regulated by reversible acetylation. Science 293, 1653-1657 (2001).
102. S. Sanjabi et al., A c-Rel subdomain responsible for enhanced DNA-binding affinity and selective gene activation. Genes Dev 19, 2138-2151 (2005).
103. L. Yin, M. A. Lazar, The orphan nuclear receptor Rev-erbalpha recruits the N-CoR/histone deacetylase 3 corepressor to regulate the circadian Bmall gene. Mol Endocrinol 19, 1452-1459 (2005).
104. D. Dangaj et al., Cooperation between Constitutive and Inducible Chemokines Enables T Cell Engraftment and Immune Attack in Solid Tumors. Cancer Cell 35, 885-900.e810 (2019).
105. A. P. Huffman, J. H. Lin, S. I. Kim, K. T. Byrne, R. H. Vonderheide, CCL5 mediates CD40-driven CD4+ T cell tumor infiltration and immunity. JCI Insight 5 (2020).
106. B. Mlecnik et al., Biomolecular network reconstruction identifies T-cell homing factors associated with survival in colorectal cancer. Gastroenterology 138, 1429-1440 (2010).
107. H. Harlin et al., Chemokine expression in melanoma metastases associated with CD8+ T-cell recruitment. Cancer Res 69, 3077-3085 (2009).
108. M. T. Chow et al., Intratumoral Activity of the CXCR3 Chemokine System Is Required for the Efficacy of Anti-PD-1 Therapy. Immunity 50, 1498-1512.e1495 (2019).
109. A. J. Ozga, M. T. Chow, A. D. Luster, Chemokines and the immune response to cancer. Immunity 54, 859-874 (2021).
110. N. Nagarsheth, M. S. Wicha, W. Zou, Chemokines in the cancer microenvironment and their relevance in cancer immunotherapy. Nat Rev Immunol 17, 559-572 (2017).
111. A. Spaks, Role of CXC group chemokines in lung cancer development and progression. J Thorac Dis 9, S164-S171 (2017).
112. Y. Cheng, X. L. Ma, Y. Q. Wei, X. W. Wei, Potential roles and targeted therapy of the CXCLs/CXCR2 axis in cancer and inflammatory diseases. Biochim Biophys Acta Rev Cancer 1871, 289-312 (2019).
113. J. Pan et al., CXCR3/CXCR3 ligand biological axis impairs RENCA tumor growth by a mechanism of immunoangiostasis. JImmunol 176, 1456-1464 (2006).
114. Y. Nesbeth et al., CCL5-mediated endogenous antitumor immunity elicited by adoptively transferred lymphocytes and dendritic cell depletion. Cancer Res 69, 6331-6338 (2009).
115. Z. M. Khan et al., Structural basis for the action of the drug trametinib at KSR-bound MEK. Nature 588, 509-514 (2020).
116. D. V. Faget, Q. Ren, S. A. Stewart, Unmasking senescence: context-dependent effects of SASP in cancer.Nat Rev Cancer 19, 439-453 (2019).
117. I. Ischenko et al., KRAS drives immune evasion in a genetic model of pancreatic cancer. Nat Commun 12, 1482 (2021).
118. L. Li et al., HDAC3 Inhibition Promotes Antitumor Immunity by Enhancing CXCL10-Mediated Chemotaxis and Recruiting of Immune Cells. Cancer Immunol Res 11, 657-673 (2023).
119. K. H. Tang et al., Combined Inhibition of SHP2 and CXCR1/2 Promotes Antitumor T-cell Response in NSCLC. Cancer Discov 12, 47-61 (2022).
120. D. N. Sidiropoulos et al., Integrated T cell cytometry metrics for immune-monitoring applications in immunotherapy clinical trials. JCI Insight 7 (2022).
121. Y. Kim et al., Immunomodulation of HDAC Inhibitor Entinostat Potentiates the Anticancer Effects of Radiation and PD-1 Blockade in the Murine Lewis Lung Carcinoma Model. Int JMol Sci 23 (2022).
122. A. Nguyen et al., HDAC1 promotes inflammatory remodeling of the tumor microenvironment to enhance epitope spreading and antitumor immunity. J Clin Invest 132 (2022).
123. K. C. Hicks et al., Tumour-targeted interleukin-12 and entinostat combination therapy improves cancer survival by reprogramming the tumour immune cell landscape. Nat Commun 12, 5151 (2021).
124. K. K. Tsai et al., Screening of organoids derived from patients with breast cancer implicates the repressor NCOR2 in cytotoxic stress response and antitumor immunity. Nat Cancer 3, 734-752 (2022).
125. X. Lu et al., HOXB13 suppresses de novo lipogenesis through HDAC3-mediated epigenetic reprogramming in prostate cancer. Nat Genet 54, 670-683 (2022).
126. F. Skoulidis et al., STK11/LKB1 Mutations and PD-1 Inhibitor Resistance in KRAS-Mutant Lung Adenocarcinoma. Cancer Discov 8, 822-835 (2018).
127. A. Liberzon et al., The Molecular Signatures Database (MSigDB) hallmark gene set collection. Cell Syst 1, 417-425 (2015).
128. A. L. Fridman, M. A. Tainsky, Critical pathways in cellular senescence and immortalization revealed by gene expression profiling. Oncogene 27, 5975-5987 (2008).

1. A method of treating or preventing cancer in a subject comprising co-administering to the subject a Kirsten rat sarcoma (KRAS) inhibitor and a histone deacetylase (HDAC) inhibitor.

2. The method of claim 1, wherein the subject suffers from lung cancer.

3. The method of claim 2, wherein the lung cancer is a non-small cell lung cancer (NSCLC).

4. The method of claim 1, wherein the cancer is KRAS inhibitor resistant.

5. The method of claim 4, wherein the cancer is trametinib resistant.

6. The method of claim 1, wherein the cancer is KRAS, LKB1 mutant lung cancer.

7. The method of claim 1, wherein the KRAS inhibitor is selected from Sotorasib (AMG510), Adagrasib/MRTX849, AMG 404, trametinib, RMC-4630, afatinib, pembro, panitumumab, carbo/pem/docetaxel, everolimus, Palbociclib, bevacizumab, LY3537982, abemaciclib, erlotinib, sintilimab, temuterkib, LY3295668, cetuximab, JNJ-74699157 (ARS-3248), GDC-6036, atezo, spartalizumab, TNO155, EGF816 (nazertinib/EGFR TKI, RMC-4630, cobimetinib/Osimertinib, BI 1701963, MRTX1133, AMG510, and irinotecan.

8. The method of claim 7, wherein the KRAS inhibitor is trametinib.

9. The method of claim 1, wherein the HDAC inhibitor is an HDAC3 inhibitor.

10. The method of claim 1, wherein the HDAC inhibitor is selected from trichostatin A, vorinostat, givinostat, abexinostat, belinostat, panobinostat, resminostat, quisinostat, depsipeptide, entinostat, mocetinostat suberoyl bis-hydroxamic acid, scriptaid, apicidin, CBHA, CI 994, Salermide, Belinostat, KD 5170, MS-275, TC-H 106, Droxinostat, Mocetinostat, PCI-24781, Pimelic Diphenylamide 106, BRD3308, and RGFP966.

11. The method of claim 1, wherein the HDAC inhibitor is entinostat.

12-14. (canceled)

15. A pharmaceutical composition comprising a KRAS inhibitor and an HDAC inhibitor.

16. The pharmaceutical composition of claim 15, wherein the KRAS inhibitor is selected from Sotorasib (AMG510), Adagrasib/MRTX849, AMG 404, trametinib, RMC-4630, afatinib, pembro, panitumumab, carbo/pem/docetaxel, everolimus, Palbociclib, bevacizumab, LY3537982, abemaciclib, erlotinib, sintilimab, temuterkib, LY3295668, cetuximab, JNJ-74699157 (ARS-3248), GDC-6036, atezo, spartalizumab, TNO155, EGF816 (nazertinib/EGFR TKI, RMC-4630, cobimetinib/Osimertinib, BI 1701963, MRTX1133, AMG510, and irinotecan.

17. The pharmaceutical composition of claim 15, wherein the KRAS inhibitor is trametinib.

18. The pharmaceutical composition of claim 15, wherein the HDAC inhibitor is an HDAC3 inhibitor.

19. The pharmaceutical composition of claim 15, wherein the HDAC inhibitor is selected from trichostatin A, vorinostat, givinostat, abexinostat, belinostat, panobinostat, resminostat, quisinostat, depsipeptide, entinostat, mocetinostat suberoyl bis-hydroxamic acid, scriptaid, apicidin, CBHA, CI 994, Salermide, Belinostat, KD 5170, MS-275, TC-H 106, Droxinostat, Mocetinostat, PCI-24781, Pimelic Diphenylamide 106, BRD3308, and RGFP966.

20. The pharmaceutical composition of claim 15, wherein the HDAC inhibitor is entinostat.
